(19) United States
(12) Patent Application Publication
(10) Pub. No.: US 2022/0372511 A1
(43) Pub. Date: Nov. 24, 2022

(54) DNA CONSTRUCT FOR TARGETING THERAPEUTIC MOLECULES TO DISEASED TISSUE BY IMMUNE CELLS

(71) Applicant: The Regents of the University of California, Oakland, CA (US)

(72) Inventors: James G. Tidball, Los Angeles, CA (US); Michelle Webhling-Henricks, Los Angeles, CA (US); Steven Welc, Los Angeles, CA (US)

(21) Appl. No.: 17/618,292

(22) PCT Filed: Jun. 12, 2020
(86) PCT No.: PCT/US20/37462

§ 371 Date: Dec. 10, 2021

Related U.S. Application Data
(60) Provisional application No. 62/861,764, filed on Jun. 14, 2019.

(51) Int. Cl. C12N 15/86; A61K 35/28; C07K 14/715

(52) U.S. Cl. CPC C12N 15/86; A61K 35/28; C07K 14/715; C12N 2750/14143; C12N 2830/008; A61K 38/00

(57) ABSTRACT

Provided herein are polynucleotide constructs comprising a CD11b promoter operably linked to a nucleic acid encoding one or more therapeutic polypeptides, to vectors, cells, and/or compositions comprising the same, and to methods of their use.

### CROSS-REFERENCE TO RELATED APPLICATION

This application claims the benefit of U.S. Provisional Application No. 62/861,764, filed on Jun. 14, 2019, which is incorporated herein by reference in its entirety.

### STATEMENT REGARDING FEDERALLY-SPONSORED RESEARCH

This invention was made with government support under Grant Number AR066817, awarded by the National Institutes of Health. The government has certain rights in the invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 262232001840SEQLIST.TXT, date recorded: Jun. 3, 2020, size: 68 KB).

### FIELD OF THE INVENTION

The present disclosure relates to polynucleotide constructs comprising a CD11b promoter operably linked to a nucleic acid encoding one or more therapeutic polypeptides, to vectors, cells, and/or compositions comprising the same, and to methods of their use.

### BACKGROUND

Conventional delivery of drugs via systemic administration exposes all tissues to the drug, and often results in undesirable, off-target effects. Similarly, a dosage that is systemically safe may not be therapeutically maximal in the desired target tissue. Thus, there is need for DNA constructs that are expressed in specific cell types (e.g., immune cells) that naturally home to diseased tissues, leading to the specific delivery of therapeutic molecules that express in the damaged tissue while avoiding the consequences of off-target effects.

All references cited herein, including patent applications, patent publications, non-patent literature, and UniProtKB/Swiss-Prot Accession numbers are herein incorporated by reference in their entirety, as if each individual reference were specifically and individually indicated to be incorporated by reference.

### BRIEF SUMMARY

To meet the above and other needs, disclosed herein are DNA constructs comprising a CD11b promoter operably linked to a nucleic acid encoding a therapeutic polypeptide (e.g., a LIF polypeptide), where the constructs are capable of driving the expression of the encoded therapeutic polypeptide in one or more immune cells, such as myeloid cells (see e.g., Example 1 below). The present disclosure is based, at least in part, on the surprising finding that the DNA constructs described herein are biologically targeted in vivo to deliver a therapeutic, biological molecule directly to diseased tissue using immune cells as a delivery system. In some embodiments, the immune cells are genetically modified to contain the DNA construct such that the immune cells robustly produce the desired therapeutic molecule, and home specifically to the diseased tissue using the endogenous, biological processes directing inflammatory cell migration. The DNA constructs described herein allow for delivery of therapeutic molecules directly to the site of disease for maximum, beneficial impact without dangerous, off-target effects in other tissues (common observed with standard, systemic delivery of drugs).

Accordingly, in one aspect, provided herein is a polynucleotide comprising a CD11 promoter operably linked to a nucleic acid molecule encoding a therapeutic polypeptide. In some embodiments, the CD11b promoter is a human CD11b promotor or a chimpanzee CD11b promoter. In some embodiments that may be combined with any of the preceding embodiments, the CD11b promoter comprises a sequence at least 95%, at least 96%, at least 97% at least 98%, at least 99%, or 100% identical to the sequence of SEQ ID NO: 1 or 2.

In some embodiments that may be combined with any of the preceding embodiments, the nucleic acid molecule encodes a Leukemia Inhibitory Factor (LIF) polypeptide. In some embodiments, the nucleic acid molecule encoding the LIF polypeptide comprises a nucleic acid sequence at least 95%, at least 96%, at least 97%, at least 98%, at least 99%, or 100% identical to a sequence selected from the group consisting of SEQ ID NOS: 3-11. In some embodiments that may be combined with any of the preceding embodiments, the LIF polypeptide comprises an amino acid sequence at least 95%, at least 96%, at least 97%, at least 98%, at least 99%, or 100% identical to a sequence selected from the group consisting of SEQ ID NOS: 18-26. In some embodiments that may be combined with any of the preceding embodiments, the polynucleotide comprises a sequence at least 95%, at least 96%, at least 97%, at least 98%, at least 99%, or 100% identical to the sequence of SEQ ID NOS: 12 or 13.

In some embodiments that may be combined with any of the preceding embodiments, the nucleic acid molecule encodes a Klotho polypeptide. In some embodiments, the nucleic acid molecule encoding the Klotho polypeptide comprises a nucleic acid sequence at least 95%, at least 96%, at least 97%, at least 98%, at least 99%, or 100% identical to the sequence of SEQ ID NOS: 14 or 15. In some embodiments that may be combined with any of the preceding embodiments, the Klotho polypeptide comprises an amino acid sequence at least 95%, at least 96%, at least 97%, at least 98%, at least 99%, or 100% identical to the sequence of SEQ ID NOS: 27 or 28.

In some embodiments that may be combined with any of the preceding embodiments, the nucleic acid molecule encodes an IL-10 polypeptide. In some embodiments, the nucleic acid molecule encoding the IL-10 polypeptide comprises a nucleic acid sequence at least 95%, at least 96%, at least 97%, at least 98%, at least 99%, or 100% identical to the sequence of SEQ ID NOS: 16 or 17. In some embodiments that may be combined with any of the preceding embodiments, the IL-10 polypeptide comprises an amino acid sequence at least 95%, at least 96%, at least 97%, at least 98%, at least 99%, or 100% identical to the sequence of SEQ ID NOS: 29 or 30.

In some embodiments that may be combined with any of the preceding embodiments, the polynucleotide further comprises a linker sequence between the CD11b promoter and the nucleic acid encoding the therapeutic polypeptide. In some embodiments, the linker sequence comprises a nucleic acid sequence at least 95%, at least 96%, at least 97%, at least 98%, at least 99%, or 100% identical to the sequence of SEQ ID NO: 31.

In another aspect, provided herein is a vector comprising any of the polynucleotides described herein. In some embodiments, the vector is a viral vector. In some embodiments, the viral vector is an adenoviral vector or a lentiviral vector. In some embodiments that may be combined with any of the preceding embodiments, the vector is an expression vector.

In another aspect, provided herein is a population of cells comprising any of the polynucleotides and/or vectors described herein. In some embodiments, the population of cells comprises hematopoietic stem cells. In some embodiments that may be combined with any of the preceding embodiments, the population of cells comprises primary cells isolated from a subject. In some embodiments, the primary cells are hematopoietic stem cells.

In another aspect, provided herein is a composition comprising: (a) any of the polynucleotides, vectors, and/or populations of cells described herein, and (b) a pharmaceutically acceptable carrier or excipient. In some embodiments, the composition further comprises an immunosuppressant. In some embodiments, the immunosuppressant is selected from prednisone, deflazacort, cytotoxic T-lymphocyte-associated protein-4, and any combinations thereof.

In another aspect, provided herein is a method of treating a subject in need thereof, the method comprising administering an effective amount of a population of cells comprising any of the polynucleotides and/or vectors described herein to the subject. In some embodiments, the population of cells comprises cells that were isolated from a healthy donor. In some embodiments, the population of cells comprises cells that were isolated from the subject. In some embodiments that may be combined with any of the preceding embodiments, the population of cells comprises hematopoietic stem cells. In some embodiments that may be combined with any of the preceding embodiments, the population of cells are contacted with the polynucleotide or vector ex vivo. In some embodiments that may be combined with any of the preceding embodiments, at least one cell in the population of cells expresses or is capable of expressing the therapeutic polypeptide. In some embodiments that may be combined with any of the preceding embodiments, the method further comprises administering an immunosuppressant to the subject. In some embodiments, the immunosuppressant is selected from prednisone, deflazacort, cytotoxic T-lymphocyte-associated protein-4, and any combinations thereof.

In some embodiments that may be combined with any of the preceding embodiments, the subject suffers from a disease or condition selected from muscular dystrophy, polymyositis, dermatomyositis, multiple sclerosis, and autoimmune demyelination. In some embodiments, administration of the population of cells reduces one or more signs or symptoms of the disease or condition in the subject. In some embodiments that may be combined with any of the preceding embodiments, administration of the population of cells reduces inflammation in the subject. In some embodiments that may be combined with any of the preceding embodiments, administration of the population of cells reduces fibrosis in the subject. In some embodiments that may be combined with any of the preceding embodiments, one or more cells of the population of cells localizes to a site of inflammation in the subject (e.g., and expresses the therapeutic polypeptide). In some embodiments, the one or more cells are myeloid cells. In some embodiments, the one or more myeloid cells are selected from megakaryocytes, thrombocytes, erythrocytes, mast cells, myeloblasts, basophils, neutrophils, eosinophils, monocytes, macrophages, and any combinations thereof.

It is to be understood that one, some, or all of the properties of the various embodiments described above and herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a schematic of an immune cell containing an exemplary nucleic acid construct expressing the therapeutic protein LIF from the CD11b promoter.

FIG. 2A shows the relative level of expression of CD11b in activated macrophages at days 3, 5, 7, and 9.

FIG. 2B shows the relative level of mouse Leukemia Inhibitory Factor (LIF) expression at day 9 of activated mouse bone marrow-derived macrophages (BMDMs) isolated from dystrophic mice (mdx) or dystrophic mice expressing the LIF transgene driven by the CD11b promoter (LIF/mdx).

FIG. 3 shows the numbers and distribution of F4/80+, CD163+ and CD206+ macrophages in dystrophic mouse muscle (mdx; top row) or in dystrophic mice expressing the LIF transgene driven by the CD11b promoter (LIF/mdx; bottom row). The bottom row shows the relative expression of F4/80, CD163 and CD206, and shows the density of F4/80+, CD163+ and CD206+ macrophages in muscles of dystrophic mice (mdx) or dystrophic mice expressing the LIF transgene driven by the CD11b promoter (LIF/mdx).

FIG. 4A shows a schematic of an exemplary assay for measuring monocyte chemotaxis.

FIGS. 4B-4C show the relative levels of expression of molecules involved in attracting inflammatory cells into muscles (CCL2 (FIG. 4B) and CCR2 (FIG. 4C)) in muscle samples obtained from dystrophic mice (mdx) or dystrophic mice expressing the LIF transgene driven by the CD11b promoter (LIF/mdx).

FIG. 4D shows the relative levels of expression of a molecule attracting inflammatory cells into muscle (CCL2) in untreated macrophages (vehicle) or macrophages treated with LIF.

FIG. 5 shows the numbers and distribution of collagen 1 (Col 1), collagen 3 (Col 3) and collagen 5 (Col 5) in dystrophic mouse muscle (mdx; top row) or in dystrophic mice expressing the LIF transgene driven by the CD11b promoter (LIF/mdx; bottom row). The bottom row shows the relative expression of collagens 1, 3 and 5, and shows the relative quantity of collagens 1, 3 and 5 in muscles of dystrophic mice (mdx) or dystrophic mice expressing the LIF transgene driven by the CD11b promoter (LIF/mdx).

FIG. 6A shows the relative levels of expression of connective tissue growth factor and fibronectin in muscles of dystrophic mice (md) or dystrophic mice expressing the LIF transgene driven by the CD11b promoter (LIF/mdx).

FIG. 6B shows the relative level of expression of transforming growth factor beta mRNA and protein in control cells (mdx) and cells stimulated with LIF.

FIG. 6C shows the proportion of F4/80+ macrophages that express TGF-beta in muscles of dystrophic mice (mdx) or dystrophic mice expressing the LIF transgene driven by the CD11b promoter (LF/mdx).

FIG. 6D shows sections of muscles of dystrophic mice (mdx) or dystrophic mice expressing the LIF transgene driven by the CD11b promoter (LIF/mdx) labeled with antibodies to TGF-beta (red) or F4/80 (green).

FIGS. 7A-7C show that differentiation of BMCs into macrophages increases CD11b/LIF transgene expression, causing suppression of M2-biased macrophage markers. FIG. 7A shows QPCR data showing differences in the level of Cd11b expression in C57BL6 BMCs stimulated with MCSF and differentiated to macrophages for 3-9 days. Values are normalized to 3-day cultures, n=4 for each data set; * indicates significantly different from 3-day data set and # indicates significantly different from 5- and 7-day data sets at P<0.05. P-values based on ANOVA with Tukey's multiple comparison test. For all histograms in the figure, the bars indicate mean±sem. FIG. 7B shows QPCR data showing increased Lif expression in freshly-isolated BMMCs and BMDMs cultured for 9-days from CD11b/LIF transgenic mice compared to transgene negative littermate controls (WT). Data are presented as mean±sem. BMCs were isolated from three independent donors, n=3 per data set. * indicates significantly different from WT at P<0.05. P-values based on two-tailed 1-test. F-test BMDMs day 9 (P=0.0038). FIG. 7C shows QPCR analysis showing that CD11b/LIF BMDMs have increased expression of Cd68 and reduced expression of Cd163 and Arg 1. Data are presented as mean±sem, n=5 for each data set, n=4 for WT BMDMs Inos and CD11b/LIF BMDMs Arg1 data sets (P<0.05), n.d. indicates that no expression was detected. Data presented for BMDMs in FIG. 7B and FIG. 7C were isolated from a single donor animal of each genotype and cultured as n=5 technical replicates. Significant findings were verified with biological replicates of experiments from independent donors. * indicates significantly different from WT BMDMs at P<0.05. P-values based on two-tailed t-test. F-test Cd206 (P=0.0258) and 1110 (P=0.0311).

FIGS. 8A-8S show that CD11b/LiF transgene expression modulates inflammation and reduces fibrosis. FIG. 8A shows QPCR data showing Lif expression in muscles of CD11b/LIF transgenic mdx mice (LIF/mdx) and non-transgenic littermates (WT/mdx), normalized to WT/mdx. TA muscles: n=10. Diaphragm muscles: n=8 or 7 for WT/mdx and LIF/mdx data sets, respectively. * indicates significant difference versus WT/mdi (P<0.05). For all histograms, bars indicate mean±sem. FIG. 8B shows a cross-section of WT/mdx TA muscles labeled with anti-LIF. Bars=50 μm. FIG. 8C shows a cross-section of LIF/mdx TA muscles labeled with anti-LIF. Bars=50 μm. FIG. 8D shows mean fluorescence intensity (MFI) of inflammatory lesions in sections immunolabeled for LIF. * indicates significant difference from WT/mdx (n=4; P<0.05). FIG. 5E, FIG. 8F, FIG. 8G, FIG. 8H, FIG. 8I, and FIG. 8J show cross-sections of muscles from WT/mdx and LIF/mdx mice immunolabeled with antibodies to F4/80 (FIG. 8E) and CD163 (FIG. 8H). Numbers of F4/80+(FIG. 8F, FIG. 8G) and CD163+(FIG. 8I, FIG. 8J) cells were normalized to muscle volume. Labeling of F4/80+(FIG. 8E) and CD163+(FIG. 8H) cells in TA muscle from 1-month old WT/mdx muscle. Bars=100 μm. N=5 for each group, except n=4 for F4/80 WT/mdx 1- and 12-month TA, LIF/mdx 12-month TA, WT/mdx 1- and 3-month diaphragm, and CD163 WT/mdx 12-month TA data sets. (FIG. 8K, FIG. 8L, FIG. 8M, FIG. 8N, FIG. 8O, and FIG. 8P) Cross-sections of TA (FIG. 8K, FIG. 8I) and diaphragm (FIG. 8N, FIG. 8O) muscles from 12-month-old WT/mdx (FIG. 8K, FIG. 8N) and LIF/mdx (FIG. 8L, FIG. 8O) mice were immunolabeled with anti-collagen type 1. Bars=50 μm. The volume fraction of muscle occupied by collagen type 1 (FIG. 8M, FIG. 8P). N=5 for each group, except n=4 for 3-month TA. * indicates significant difference versus age-matched WT/mdx mice (P<0.05). # and Φ indicate significant difference versus 1- and 3-months-old, genotype-matched mice, respectively (P<0.05). P-values based on two-tailed t-test. FIG. 8Q, FIG. 8R, and FIG. 8S show the passive mechanical properties of TA muscles of WT/mdx (curves 1 and 2) and LIF/mdx (curves 3-5) mice measured in-situ. Lissajous curves (FIG. 8Q) show passive stiffness (FIG. 8R) and energy dissipation (FIG. 8S) of TAs. N=2 and 3 for WT/mdx and LIF/mdx groups, respectively. * indicates significant difference versus WT/mdx mice. P-values based on two-tailed t-test.

FIGS. 9A-9L show that transplantation of CD11b/LIF transgenic BMCs into mdx mice reduces inflammation in dystrophic muscle. FIG. 9A shows QPCR analysis showing that the transplantation of CD11b/LIF transgenic BMCs into mdx recipients (LIF BMT/mdx) reduced expression of transcripts associated with M2-biased macrophages (Cd163, Cd206 and Arg2), Th2 cytokines (114 and 1110) and increased expression of the negative regulator of cytokine signaling (Socs3) compared to WT BMT mdx recipients (WT BMT/mdx) 4-months post-transplantation. N=7 or 8 for WT BMT/mdx and LIF BMT/mdx data sets, respectively, except n=7 for LIF BMT/mdx Arg1 data set. * indicates significantly different from WT BMT/mdx recipients at P<0.05. F-test Ifng (P=0.0145), 116 (P<0.0001), Il4 (P=0.0015), Il10 (P<0.0001) and Socs3 (P=0.0061). For all histograms in the figure, the bars indicate mean±sem. FIG. 9B, FIG. 9C, FIG. 9D, FIG. 9E, FIG. 9F, FIG. 9G, FIG. 9H, FIG. 9I, and FIG. 9J show cross-sections of TA muscles from WT BMT/mdx (FIG. 9C, FIG. 9F, FIG. 9I) or LIF BMT/mdx (FIG. 9D, FIG. 9G, FIG. 9J) mice immunolabeled with antibodies to F4/80 (FIG. 9C, FIG. 9D), CD163 (FIG. 9F, FIG. 9G), and CD206 (FIG. 9I, FIG. 9J). Bars=50 μm. The numbers of F4/80+(FIG. 9B), CD163+(FIG. 9E), and CD206+(FIG. 9H) cells normalized to muscle volume were reduced in LIF BMT/mdx recipients. Similarly, cross-sections were immunolabeled with antibodies to CD4 and Ly-6B.2 (neutrophils) to test for changes in the concentrations of other populations of immune cells. There was no change in the concentrations of CD4+(FIG. 9K) and Ly-6B.2+ (FIG. 9L) cells. N=5 for each data set, except n=4 for CD206 LIF BMT/mdx data set. * indicates significantly different from WT BMT/mdx recipients at P<0.05. All P-values based on two-tailed t-test.

FIGS. 10A-10G show that transplantation of CD11b/LIF transgenic BMCs disrupts Ccl2 expression in dystrophic muscles by inhibiting macrophage expression of CCL2. FIG. 10A shows QPCR analysis shows that TA muscles from LIF BMT/mdx recipients have reduced expression of Ccr2 and its ligands Ccl2, Ccl7 (P=0.06), Ccl8 and Ccl12. N=7 or 8 for WT BMT/mdx and LIF BMT/mdx data sets, respectively, except n=7 for LIF BMT/mdx Ccl8 data set. * indicates significantly different from WT BMT/mdx recipients at P<0.05. F-test Ccr2 (P=0.0087), Ccl2 (P<0.0001). Ccl7 (P=0.0001), Ccl8 (P=0.001) and Ccl12 (P=0.001). For all histograms in the figure, the bars indicate mean±sem. FIG. 10B shows QPCR analysis for Ccr2 gene expression of BMDMs treated with recombinant LIF (10 ng/ml) for 3- and 24-hours. FIG. 10C and FIG. 10D show muscle sections co-labeled with antibodies to CD68 (FIG. 10C) or CD206 (FIG. 10D) and CCR2 showing no change in the proportions of cells co-expressing CCR2 between transplant recipient groups. N=5 for each data set. FIG. 10E shows QPCR analysis showing reduced Cc/2 gene expression in BMDMs stimulated with LIF as described in FIG. 10B. FIG. 10F shows ELISA of conditioned media showing less CCL2 secreted into the media of BMDMs stimulated with LIF for 6- and 24-hours compared to control cultures. For cell culture experiments, N=5 technical replicates for each data set, cells for each time point were isolated from independent donors. Significant findings were verified with biological replicates of experiments from independent donors. P-values based on two-tailed t-test. F-test CCL2 protein 24-hours (P=0.0337). FIG. 10G shows a cross-section of TA muscles from WT BMT/mdx or LIF BMT/mdx mice that were immunolabeled with antibodies to F4/80 (green) and CCL2 (red) and shows that F4/80+ cells express CCL2. Nuclei are stained blue with DAPI. Bar=10 μm. FIG. 10H shows the proportion of F4/80+ cells co-expressing CCL2 was reduced in LIF BMT/mdx recipients. N=5 for each data set. * indicates significantly different from WT BMT/mdx recipients at P<0.05. P-values based on two-tailed t-test.

FIGS. 11A-11K show that transplantation of CD11b/LIF transgenic BMCs into mdx mice reduces muscle fibrosis. FIG. 11A, FIG. 11B, FIG. 11C, FIG. 11D, FIG. 11E, FIG. 11F, FIG. 11G, FIG. 11H, and FIG. 11I show TA muscles from WT BMT/mdx (in FIG. 11B, FIG. 11E, and FIG. 11H) and LIF BMT/mdx transplant recipients (in FIG. 11C, FIG. 11F, and FIG. 11I) immunolabeled for collagen types 1 (FIG. 11A, FIG. 11B, and FIG. 11C), 3 (FIG. 11D, FIG. 11E, FIG. 11F) and 5 (FIG. 11G, FIG. 11H, and FIG. 11I). Bars=50 μm. The volume fraction of muscle occupied by collagen types 1 (FIG. 11A), 3 (FIG. 11D) and 5 (FIG. 11G) were reduced in LIF BMT/mdx recipients. N=5 for WT BMT/mdx and LIF BMT/mdx data sets, except n=4 WT BMT/mdx collagen type 3 and LIF BMT/mdx collagen type 1. * indicates significantly different from WT BMT/mdx recipients at P<0.05. F-test collagen type 3 (P=0.0055) and type 5 (P=0.0155). For all histograms in the figure, the bars indicate mean±sem. FIG. 11J shows QPCR data presented as mean±sem shows that LIF BMT/mdx recipients also had reduced expression of transcripts encoding Col1a1, Col3a1 and Col5a3. N=7 or 8 for WT BMT/mdx and LIF BMT/mdx data sets, respectively, except n=5 for Col5a3 data sets. FIG. 11K shows QPCR analysis of transcripts associated with the pro-fibrotic Wnt- (Axin2) and TGFβ1-signaling (Tgfb1, Cgf, Fn1 and Snai1) pathways showed reduced expression of Ctgf, Fn1 and Snai1 in LIF BMT/mdx recipients. N=7 or 8 for WT BMT/mdx and LIF BMT/mdx data sets, respectively, except n=7 for LIF BMT/mdx Axin2 group. * indicates significantly different from WT BMT/mdx recipients at P<0.05. P-values based on two-tailed t-test. F-test Col1a1 (P=0.0250).

FIGS. 12A-12E show that LIF inhibits macrophage TGFβ1 expression. FIG. 12AB, FIG. 12B, and FIG. 12C show muscle sections co-labeled with antibodies to pro-fibrotic TGFβ (red) and the pan macrophage marker F4/80 (green) to test for changes in macrophage expression of TGFβ. Nuclei appear blue (DAPI). Bars=25 μm. FIG. 12A shows that the proportion of F4/80+ cells co-expressing TGFβ was reduced in LIF BMT/mdx recipients. FIG. 12B shows WT BMT/mdx recipient muscle sections co-labeled with antibodies to pro-fibrotic TGFβ (red), the pan macrophage marker F4/80 (green), and TGFβ (orange). FIG. 12C shows LIF BMT/mdx recipient muscle sections co-labeled with antibodies to pro-fibrotic TGFβ (red), the pan macrophage marker F4/80 (green), and TGFβ (orange). The greatest reduction in the number of F4/80+ cells positive for TGFβ (orange) were in inflammatory lesions of LIF BMT/mdx (FIG. 12C) compared to WT BMT/mdx recipients (FIG. 12B). N=5 for each data set, * indicates significantly different from WT BMT/mdx recipients at P<0.05. For all histograms in the figure, the bars indicate mean±sem. FIG. 12D shows QPCR analysis of BMDMs treated with recombinant LIF (10 ng/ml) for 3- or 24-hours showing that Tgfb1 expression is inhibited by LIF after 24-hours of stimulation. FIG. 12E shows that the concentration of secreted TGFβ was also reduced in BMDMs stimulated with LIF for 24-hours, analyzed by ELISA. N=5 technical replicates for each data set. Significant findings were verified with biological replicates of experiments from independent donors. * indicates significantly different from control at P<0.05.

FIGS. 13A-13J show that LIF inhibits fibrogenesis and TGFβ1-induced Ctgf expression in muscle cells. FIG. 13A shows TA muscle sections co-labeled with antibodies to Pax7 (red) and HSP47 (green) in WT BMT/mdx (FIG. 13A) and LIF BMT/mdx (FIG. 13B) recipients. Nuclei appear blue (DAPI). Bars=5 μm. FIG. 13C shows that fewer Pax7+ cells co-expressed HSP47 in LIF BMT/mdx recipients (green symbols) compared to WT BMT/mdx recipients (black symbols). FIG. 13D shows muscle sections co-labeled with antibodies to Pax7 and fibrogenic marker Ertr7 to confirm that fewer Pax7+ cell acquired a fibrogenic phenotype in LIF BMT/mdx recipients. N=5 for each data set, except n=4 for WT BMT/mdx Pax7/HSP47 data set, * indicates significantly different from WT BMT/mdx at P<0.05. P-values based on two-tailed t-test. For all histograms in the figure, the bars indicate mean±sem. FIG. 13E, FIG. 13F, FIG. 13G, FIG. 13H, FIG. 13I, and FIG. 13J show Myoblasts (black symbols) and myotubes (green symbols) were stimulated with LIF (10 ng/ml) and TGFβ1 (10 ng/ml) for 3- (FIG. 13E, FIG. 13F, and FIG. 13G) or 24-hours (FIG. 13H, FIG. 13I, and FIG. 13J). FIG. 13E and FIG. 13H show LIF inhibited TGFβ1-induced Ctgf mRNA in myoblasts and myotubes after 3- and 24-hours of stimulation. LIF inhibited basal Ctgf expression in myotubes at 24-hours (FIG. 13H). FIG. 13F and FIG. 13I show that LIF did not affect Fn1 expression in myoblasts or myotubes after 3- or 24-hours. Additionally, LIF attenuated TGFβ1-induced Col1a1 expression in myotubes, but not myoblasts after 3-hours of stimulation (FIG. 13G). Myoblasts stimulated with LIF for 24-hours had reduced Col1a1 expression (FIG. 13j). N=4 technical replicates per group. Significant findings were verified with biological replicates of experiments from independent cultures. * indicates significantly different from control, # indicates significantly different from TGFβ1-stimulated and 4 indicates significantly different from LIF-stimulated at P<0.05. P-values based on ANOVA with Tukey's multiple comparison test.

FIGS. 14A-14K show that transplantation of CD11b/LIF transgenic BMCs reduces the numbers of FAPs in dystrophic muscle but does not affect phenotype. FIG. 14A shows QPCR analysis showing that TA muscles from LIF BMT/mdx recipients have reduced Pdgfra gene expression. N=7 or 8 for WT BMT/mdx and LIF BMT/mdx data sets, respectively, * indicates significantly different from WT BMT/mdx recipients at P<0.05. P-values based on two-tailed t-test. For all histograms in the figure, the bars indicate mean±sem. To quantify the number of FAPs, FIG. 14B shows muscle sections co-labeled with antibodies to PDGFRα (red) and CD31, CD45 (green). Arrowheads indicate FAPs (CD31−CD45−PDGFRα+). Bar=50 μm. FIG. 14C shows that fewer FAPs (CD31−CD45−PDGFRα+) in TA cross-sections of LIF BMT/mdx recipients compared to WT BMT/mdx recipients. N=5 for each data set. FIG. 14D shows that there was no detectible change in phenotype of PDGFRα+ cells assayed for co-expression of the fibrogenic marker HSP47. FIG. 14E shows FACS plots demonstrating strategy for sorting FAPs (Hoechst+CD11b−CD31−CD45−PDGFRα+). Fibroblasts-derived from FAPs were stimulated with LIF (10 ng/ml) and/or TGFβ1 (10 ng/ml) for 3-hours (FIG. 14F, FIG. 14G, and FIG. 14H) or 3-days (FIG. 14I, FIG. 14J, and FIG. 14K) and assayed by QPCR for Ctgf (FIG. 14F, and FIG. 14I). Fn1 (FIG. 14G, and FIG. 14J) and Col1a1 (FIG. 14H, and FIG. 14K). N=4 technical replicates for each data set. Significant findings were verified with biological replicates of cells sorted from independent donors. * indicates significantly different from control cultures, # indicates significantly different from TGFβ1 treated cultures, and 4 indicates significantly different from LIF-treated cultures at P<0.05. P-values based on ANOVA with Tukey's multiple comparison test.

FIGS. 15A-15E show that transplantation of CD11b/LIF transgenic BMCs does not affect muscle growth or regeneration. Assays of muscle mass to body mass ratio (FIG. 15A), fiber number (FIG. 15B), proportion of centrally-nucleated regenerating fibers (FIG. 15C), and muscle fiber cross-sectional area (FIG. 15D) indicate no difference in muscle growth or regeneration between WT BMT/mdx and LIF BMT/mdx recipients. N=5 per group. For all histograms in the figure, the bars indicate mean±sem. FIG. 15E shows QPCR analysis showing no difference in the expression of myogenic transcription factors (Pax7, Myod1, Myog and Mrf4) in WT BMT/mdx versus LIF BMT/mdx recipients. N=7 or 8 for WT BMT/mdx and LIF BMT/mdx data sets, respectively. No significant differences were identified between groups at P<0.05, determined by two-tailed t-test.

FIG. 16 shows potential immunomodulatory and anti-fibrotic actions of LIF expressed by the CD11b/LIF transgene in muscular dystrophy. 1. LIF can serve an immunomodulatory role by reducing the expression of Ccl2 in macrophages, which is associated with reduced recruitment of monocytes/macrophages into dystrophic muscle. 2. LIF can serve an immunomodulatory role by reducing the activation of monocytes/macrophages to a CD163+, M2-biased phenotype that can increase fibrosis of dystrophic muscle. 3. LIF can reduce the expression of the pro-fibrotic molecules Arg1 and Tgfb1 in macrophages. 4. LIF can reduce the TGFβ1-mediated induction of pro-fibrotic genes in muscle cells, including Ctgf and Col1a1.

FIGS. 17A-17D show that expression of a CD11b/LIF transgene in mdx mice does not affect systemic pro-inflammatory or anti-inflammatory cytokine expression in mdx mice. Serum ELISA for circulating levels of IFNγ (FIG. 17A), TNF (FIG. 17B), IL-4 (FIG. 17C) and IL-10 (FIG. 17D) showed no significant change in serum cytokine concentrations of 3-months old WT/mdx and LiF/mdx mice. For all histograms in the figure, the bars indicate mean±sem. N=3 for each data set. No significant differences were identified between groups using P-values <0.05, determined by two-tailed t-test.

FIG. 18A and FIG. 18B show that expression of a CD11b/LIF transgene in mdx mice does not affect CD68+ cell numbers in TA or diaphragm muscle. TA (FIG. 18A) and diaphragm (FIG. 18B) muscles of WT/mdx and LIF/mdx transgenic mice were immunolabeled and the numbers of CD68+ cells at the ages of 1-, 3- and 12-months were quantified. No significant differences in the number of CD68+ cells were found between genotypes at the ages tested. For all histograms in the figure, the bars indicate mean±sem. N=5 for each data set, except n=4 for 3-months WT/mdx mice. # indicates significant difference versus 1-month mice of the same genotype at P<0.05. P-values based on two-tailed t-test.

FIGS. 19A-19D show that expression of a CD11b/LIF transgene in mdx mice reduces collagen types 3 and 5 accumulation in TA and diaphragm muscles. For FIG. 19A, FIG. 19B, FIG. 19C, and FIG. 19D, TA and diaphragm muscles of WT/mdx and LIF/mdx mice were immunolabeled for collagen types 3 (FIG. 19A, FIG. 19C) or 5 (FIG. 19B, FIG. 19D) at the ages of 1-, 3- and 12-months. The volume fraction of area occupied by each collagen type was quantified in the TA (FIG. 19A, FIG. 19B) and diaphragm (FIG. 19C, FIG. 19D). The accumulation of collagen type 3 was reduced in 1- and 12-months diaphragms of LiF/mdx mice. Collagen type 5 accumulation was also reduced in 3-months TAs, 1- and 12-months diaphragms of LIF/mdx mice. For all histograms in the figure, the bars indicate mean±sem. N=5 for each data set, except n=4 for collagen type 3 WT/mdx 1- and 3-month TA, LIF/mdx 3-month TA, WT/mdx 3-month diaphragm data sets. * indicates significant difference versus WT/mdx mice of the same age at P<0.05. # indicates significant difference versus 1-month mice of the same genotype at P<0.05. Φ indicates significant difference versus 3-months-old mice of the same genotype at P<0.05. P-values based on two-tailed t-test. F-test collagen type 3 3-month diaphragm (P=0.0242) and collagen type 5 3-month TA (P=0.0139).

FIGS. 20A-20C show that expression of a CD11b/LIF transgene in mdx mice does not affect muscle fiber growth but increases the formation of regenerating fibers. FIG. 20A shows the average fiber cross-sectional area of WT/mdx and LIF/mdx TAs quantified at 1-, 3- and 12-months. No significant differences were detected between the two genotypes at the ages tested. For all histograms in the figure, the bars indicate mean±sem. N=5 for each data set, except n=4 for 3- and 12-months LIF/mdx TA muscles. # indicates significant difference versus 1-month mice of the same genotype at P<0.05. Φ indicates significant difference versus 3-months-old-mice of the same genotype at P<0.05. P-values based on two-tailed t-test. F-test TA muscle fiber cross-sectional area 3- (P=0.0222) and 12-months TA (P=0.0230). FIG. 20B and FIG. 20C show TA and diaphragm muscles of WT/mdx and LIF/mdx mice immunolabeled with antibodies to developmental myosin heavy chain (dMHC) at 1-, 3- and 12-months. The proportion of dMHC+ to total muscle fibers was quantified. The proportion of dMHC+ fibers increased in TA muscles at 3-months (FIG. 20B) and diaphragm muscles at 3- and 12-months (FIG. 20C). Data are presented as mean±sem, n=5 for each data set, except n=4 for 1-, 3- and 12-months WT/mdx TA muscles and 3- and 12-months WT/mdx diaphragm muscles. * indicates significant difference versus WT/mdx mice of the same age at P<0.05. P-values based on two-tailed t-test. F-test diaphragm muscle fiber cross-sectional area 3-months (P=0.0113).

FIGS. 21A-21F show that expression of a CD11b/LIF transgene in mdx mice attenuates expression of fibrogenic genes in myogenic progenitor cells. FIG. 21A shows FACS plots demonstrating strategy for sorting myogenic progenitor cells (DAPI-CD11b-CD31-CD45-Sca1-Intα7+) from 14-months old WT/mdx and LIF/mdx mice. FIG. 21B, FIG. 21C, FIG. 21D, FIG. 21E, and FIG. 21F show RNA collected from sorted myogenic progenitor cells and used for QPCR analysis of Fn (FIG. 21B), Col3a1 (FIG. 21C), Hsp47 (FIG. 21D), Col1a1 (FIG. 21E) and Ctgf (FIG. 21F). For all histograms in the figure, the bars indicate mean±sem. N=3 for each data set. * indicates significant difference versus WT/mdx mice at P<0.05. P-values based on two-tailed t-test.

### DETAILED DESCRIPTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle. J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994): Short Protocols in Molecular Biology (Wiley and Sons, 1999).

### II. Definitions

Before describing the present disclosure in detail, it is to be understood that this present disclosure is not limited to particular compositions or biological systems, which can, of course, vary. It is also to be understood that the terminology used herein is for the purpose of describing particular embodiments only, and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure described herein include “comprising.” “consisting,” and “consisting essentially of” aspects and embodiments.

The term “and/or” as used herein a phrase such as “A and/or B” is intended to include both A and B; A or B; A (alone): and B (alone). Likewise, the term “and/or” as used herein a phrase such as “A, B, and/or C” is intended to encompass each of the following embodiments: A, B, and C: A, B, or C: A or C; A or B: B or C: A and C: A and B; B and C; A (alone); B (alone); and C (alone).

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbanates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′. CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “isolated nucleic acid” refers to a nucleic acid molecule of genomic, cDNA, or synthetic origin, or a combination thereof, which is separated from other nucleic acid molecules present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid of interest).

As used herein, a nucleic acid is “operatively linked” or “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operatively linked” or “operably linked” mean that the DNA sequences being linked are contiguous.

The term “vector” refers to a nucleic acid molecule capable of transporting a heterologous or foreign nucleic acid molecule. The heterologous or foreign nucleic acid molecule is linked to the vector nucleic acid molecule by a recombinant technique, such as ligation or recombination. This allows the heterologous or foreign nucleic acid molecule to be multiplied, selected, further manipulated or expressed in a host cell or organism. A vector can be a plasmid, phage, transposon, cosmid, chromosome, virus, or virion. One type of vector can be integrated into the genome of a host cell upon introduction into the host cell, and thereby is replicated along with the host genome (e.g., non-episomal mammalian vectors). Another type of vector is capable of autonomous replication in a host cell into which it is introduced (e.g., episomal mammalian vectors). A specific type of vector capable of directing the expression of expressible heterologous or foreign nucleic acids to which they are operably linked is commonly referred to as “expression vectors”. Expression vectors generally have control sequences that drive expression of the expressible heterologous or foreign nucleic acids. The term “vector” encompasses all types of vectors regardless of their function.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The term “amino acid analogs” refers to compounds that have the same basic chemical structure as a naturally occurring amino acid but the C-terminal carboxy group, the N-terminal amino group, or side chain functional group has been chemically modified to another functional group. The term “amino acid mimetics” refers to chemical compounds that have a structure that is different from the general chemical structure of an amino acid, but that functions similarly to a naturally occurring amino acid.

As used herein, the twenty conventional amino acids and their abbreviations follow conventional usage. See e.g., Immunology—A Synthesis (2nd Edition. E. S. Golub and D. R. Gren, Eds., Sinauer Associates, Sunderland, Mass. (1991)).

The terms “polypeptide,” “protein.” and “peptide” are used interchangeably herein and may refer to polymers of two or more amino acids.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see e.g., Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucleic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

The terms “Leukemia Inhibitory Factor” or “LIF” are used in the present application, and includes the human LIF (e.g., UniProt accession number P15018), as well as variants, isoforms, and species homologs thereof (e.g., mouse LIF (UniProt accession number P09056), rat LIF (UniProt accession number P17777), dog LIF (UniProt accession number J9NRL6), bovine LIF (UniProt accession number Q27956), chimpanzee LIF (UniProt accession number H2QLG8), etc.).

The term “Klotho” is used in the present application, and includes the human Klotho (e.g., UniProt accession number Q9UEF7), as well as variants, isoforms, and species homologs thereof (e.g., mouse Klotho (UniProt accession number O35082), rat Klotho (UniProt accession number Q9Z2Y9), cynomolgus monkey Klotho (UniProt accession number Q8WP17), etc.).

The terms “interleukin 10”, “IL-10”, or “IL10” are used in the present application, and includes the human IL-10 (e.g., UniProt accession number P22301), as well as variants, isoforms, and species homologs thereof (e.g., mouse IL-10 (UniProt accession number P18893), rat IL-10 (UniProt accession number P29456), chicken IL-10 (UniProt accession number Q6A2H4), rabbit IL-10 (UniProt accession number Q9TSJ4), rhesus macaque IL-10 (UniProt accession number P51496), etc.).

The terms “cell” or “host cell” may refer to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; human cells (e.g., HEK293F cells, HEK293T cells; or human tissues or hybridoma cells); yeast cells; insect cells (e.g., S2 cells); bacterial cells (e.g., E, coli cells); and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the terms “cell” or “host cell.”

The term “mammal” refers to any animal species of the Mammalia class. Examples of mammals include: humans; laboratory animals such as rats, mice, hamsters, rabbits, non-human primates, and guinea pigs; domestic animals such as cats, dogs, cattle, sheep, goats, horses, and pigs; and captive wild animals such as lions, tigers, elephants, and the like.

The term “prevent” or “preventing.” with reference to a certain disease condition in a mammal, refers to preventing or delaying the onset of the disease, or preventing the manifestation of clinical or subclinical symptoms thereof.

The term “treat”, “treating”, or “treatment”, with reference to a certain disease condition in a mammal, refers to causing a desirable or beneficial effect in the mammal having the disease or condition. The desirable or beneficial effect may include reduced frequency or severity of one or more symptoms of the disease, or arrest or inhibition of further development of the disease, condition, or disorder. The effect can be either subjective or objective. For example, if the mammal is human, the human may note improved vigor or vitality or decreased pain as subjective symptoms of improvement or response to therapy. Alternatively, the clinician may notice a decrease in one or more symptoms and/or physical manifestations based on physical exam, laboratory parameters, markers or radiographic findings. Alternatively, other tests can be used to evaluate objective improvement, such as sonograms, nuclear magnetic resonance testing and positron emissions testing.

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present disclosure, an effective amount of a polynucleotide, vector, composition, or method as described herein is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

### III. Polynucleotides Suitable for Expressing a Therapeutic Molecule

Certain aspects of the present disclosure relate to polynucleotides comprising a CD11b promoter (e.g., a human CD11b promoter) operably linked to a nucleic acid sequence encoding a therapeutic polypeptide. In some embodiments, the therapeutic polypeptide is a LIF polypeptide (e.g., a human LIF polypeptide), a Klotho polypeptide (e.g., a human Klotho polypeptide), or an IL-10 polypeptide (e.g., a human IL-10 polypeptide). In some embodiments, the polynucleotides of the present disclosure are incorporated into a vector (e.g., a viral vector such as an adenovirus vector or a lentivirus vector).

### CD11b Promoters

In some embodiments, the present disclosure relates to a polynucleotide comprising a CD11b promoter operably linked to a nucleic acid molecule encoding a therapeutic polypeptide. In some embodiments, the CD11b promoter is suitable for driving expression of the therapeutic polypeptide in one or more cells that naturally and/or endogenously express CD11b (e.g., one or more myeloid cells). Examples of cells that naturally express CD11b include, without limitation, megakaryocytes, thrombocytes, erythrocytes, mast cells, myeloblasts, basophils, neutrophils, eosinophils, monocytes, macrophages, and any combinations thereof.

The CD11b promoter of the present disclosure may comprise the sequence of any CD11b promoter described herein or known in the art, including, for example, a human CD11b promoter, a chimpanzee CD11b promoter, etc.

In some embodiments, a CD11b promoter of the present disclosure comprises the nucleic acid sequence of a human CD11b promoter. In some embodiments, the CD11b promoter comprises a fragment of the human CD11b promoter (e.g., a 550 base pair fragment of the human CD11b promoter between bases −500 and +50 of the human CD11b sequence). In some embodiments, the CD11b promoter comprises a nucleic acid sequence having at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 1. In some embodiments, the CD11b promoter comprises the nucleic acid sequence of SEQ ID NO: 1. In some embodiments, the CD11b promoter comprises a 5′ truncation, a 3′ truncation, or a fragment of the sequence of SEQ ID NO: 1. For example, a CD11b promoter comprising a 5′ truncation, a 3′ truncation, or a fragment of the sequence of SEQ ID NO: 1 may include a nucleic acid molecule having at least 25, at least 50, at least 75, at least 100, at least 125, at least 150, at least 175, at least 200, at least 225, at least 250, at least 275, at least 300, at least 325, at least 350, at least 375, at least 400, at least 425, at least 450, at least 475, at least 500, or at least 525, but fewer than 550, consecutive nucleotides of SEQ ID NO: 1.

In some embodiments, a CD11b promoter of the present disclosure comprises the nucleic acid sequence of the chimpanzee CD11b promoter. In some embodiments, the CD11b promoter comprises a fragment of the chimpanzee CD11b promoter (e.g., a 213 base pair fragment of the chimpanzee CD11b promoter). In some embodiments, the CD11b promoter comprises a nucleic acid sequence having at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 2. In some embodiments, the CD11b promoter comprises the nucleic acid sequence of SEQ ID NO: 2. In some embodiments, the CD11b promoter comprises a 5′ truncation, a 3′ truncation, or a fragment of the sequence of SEQ ID NO: 2. For example, a CD11b promoter comprising a 5′ truncation, a 3′ truncation, or a fragment of the sequence of SEQ ID NO: 2 may include a nucleic acid molecule having at least 25, at least 50, at least 75, at least 100, at least 125, at least 150, at least 175, or at least 200, but fewer than 213, consecutive nucleotides of SEQ ID NO: 2.

### LIF Polypeptides

In some embodiments, the present disclosure relates to a polynucleotide comprising a CD11b promoter operably linked to a nucleic acid molecule encoding a Leukemia Inhibitory Factor (LIF) polypeptide. The nucleic acid molecule encoding a LIF polypeptide may be a nucleic acid molecule encoding any LIF polypeptide described herein or known in the art, including, for example, a nucleic acid molecule encoding a human LIF polypeptide, a nucleic acid molecule encoding a mouse LIF polypeptide, a nucleic acid molecule encoding a bovine LIF polypeptide, a nucleic acid molecule encoding a rat LIF polypeptide, etc. Exemplary nucleic acid molecules encoding LIF polypeptides are provided as SEQ ID NOS: 3-11. Nucleic acids of the present disclosure that encode a LIF polypeptide also include nucleic acids having at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence of any one of SEQ ID NOS: 3-11. In some embodiments, the nucleic acid comprises a sequence at least 90%, at least 91%, at least 92%, at least 93%, at least 94%, at least 95%, at least 96%, at least 97%, at least 98%, at least 99%, or 100% identical to the sequence of SEQ ID NO: 3.

In some embodiments, a nucleic acid encoding a LIF polypeptide is a nucleic acid that encodes an N-terminal truncation, a C-terminal truncation, or a fragment of any of the LIF polypeptides described herein or known in the art. For example, a nucleic acid encoding an N-terminal truncation, a C-terminal truncation, or a fragment of a human LIF polypeptide may be a nucleic acid having at least 25, at least 50, at least 75, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, but fewer than 609, consecutive nucleotides of SEQ ID NO: 3.

In some embodiments, the LIF polypeptide is any of the LIF polypeptides described herein or known in the art, including, for example, a human LIF polypeptide, a mouse LIF polypeptide, a rat LIF polypeptide, a dog LIF polypeptide, a non-human primate LIF polypeptide (such as a chimpanzee polypeptide), etc. Exemplary amino acid sequences of LIF polypeptides include the amino acid sequences of any of SEQ ID NOS: 18-26. In some embodiments, a nucleic acid encoding a LIF polypeptide is a nucleic acid that encodes a polypeptide having at least 75%, at least 80%, at least 85%, at least 90%, at least 91%, at least 92%, at least 93%, at least 94%, at least 95%, at least 96%, at least 97%, at least 98%, at least 99%, or 100% amino acid sequence identity to the sequence of any one of SEQ ID NOS: 18-26. In some embodiments, the LIF polypeptide comprises a sequence at least 90%, at least 91%, at least 92%, at least 93%, at least 94%, at least 95%, at least 96%, at least 97%, at least 98%, at least 99%, or 100% identical to the amino acid sequence of SEQ ID NO: 18. In some embodiments, the present disclosure relates to polynucleotides that encode polypeptides that are homologs of the H. sapiens LIF. Methods of identifying polypeptides that are homologs of a polypeptide of interest are well known to one of skill in the art.

In some embodiments, a nucleic acid encoding a LIF polypeptide is a nucleic acid that encodes N-terminal truncations, C-terminal truncations, or fragments of the amino acid sequence of any one of SEQ ID NOS: 18-26. For example, an N-terminal truncation, C-terminal truncation, or fragment of a human LIF polypeptide may comprise at least 10, at least 12, at least 14, at least 16, at least 18, at least 20, at least 30, at least 40, at least 50, at least 75, at least 100, at least 125, at least 150, at least 175, at least 200, but fewer than 202, consecutive amino acids of SEQ ID NO: 3.

### Klotho Polypeptides

In some embodiments, the present disclosure relates to a polynucleotide comprising a CD11b promoter operably linked to a nucleic acid molecule encoding a Klotho polypeptide. The nucleic acid molecule encoding a Klotho polypeptide may be a nucleic acid molecule encoding any Klotho polypeptide described herein or known in the art, including, for example, a nucleic acid molecule encoding a human Klotho polypeptide, a nucleic acid molecule encoding a mouse Klotho polypeptide, etc. Exemplary nucleic acid molecules encoding Klotho polypeptides are provided as SEQ ID NOS: 14 and 15. Nucleic acids of the present disclosure that encode a Klotho polypeptide also include nucleic acids having at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NOS: 14 or 15. In some embodiments, the nucleic acid comprises a sequence at least 90%, at least 91%, at least 92%, at least 93%, at least 94%, at least 95%, at least 96%, at least 97%, at least 98%, at least 99%, or 100% identical to the sequence of SEQ ID NO: 14.

In some embodiments, a nucleic acid encoding a Klotho polypeptide is a nucleic acid that encodes an N-terminal truncation, a C-terminal truncation, or a fragment of any of the Klotho polypeptides described herein or known in the art. For example, a nucleic acid encoding an N-terminal truncation, a C-terminal truncation, or a fragment of a human LIF polypeptide may be a nucleic acid having at least 25, at least 50, at least 75, at least 100, at least 150, at least 200, at least 250, at least 500, at least 750, at least 1000, at least 1250, at least 1500, at least 1750, at least 2000, at least 2250, at least 2500, at least 2750, or at least 3000, but fewer than 3039, consecutive nucleotides of SEQ ID NO: 14.

In some embodiments, the Klotho polypeptide is any of the Klotho polypeptides described herein or known in the art, including, for example, a human Klotho polypeptide, a mouse Klotho polypeptide, etc. Exemplary amino acid sequences of Klotho polypeptides include the amino acid sequences of SEQ ID NOS: 27 and 28. In some embodiments, a nucleic acid encoding a Klotho polypeptide is a nucleic acid that encodes a polypeptide having at least 75%, at least 80%, at least 85%, at least 90%, at least 91%, at least 92%, at least 93%, at least 94%, at least 95%, at least 96%, at least 97%, at least 98%, at least 99%, or 100% amino acid sequence identity to the sequence of SEQ ID NOS: 27 or 28. In some embodiments, the Klotho polypeptide comprises a sequence at least 90%, at least 91%, at least 92%, at least 93%, at least 94%, at least 95%, at least 96%, at least 97%, at least 98%, at least 99%, or 100% identical to the amino acid sequence of SEQ ID NO: 27. In some embodiments, the present disclosure relates to polynucleotides that encode polypeptides that are homologs of the H. sapiens Klotho. Methods of identifying polypeptides that are homologs of a polypeptide of interest are well known to one of skill in the art.

In some embodiments, a nucleic acid encoding a Klotho polypeptide is a nucleic acid that encodes N-terminal truncations, C-terminal truncations, or fragments of the amino acid sequence of SEQ ID NOS: 27 or 28. For example, an N-terminal truncation, C-terminal truncation, or fragment of a human Klotho polypeptide may comprise at least 10, at least 12, at least 14, at least 16, at least 18, at least 20, at least 30, at least 40, at least 50, at least 75, at least 100, at least 150, at least 200, at least 250, at least 500, at least 750, or at least 1000, but fewer than 1012, consecutive amino acids of SEQ ID NO: 27.

### IL-10 Polypeptides

In some embodiments, the present disclosure relates to a polynucleotide comprising a CD11b promoter operably linked to a nucleic acid molecule encoding an IL-10 polypeptide. The nucleic acid molecule encoding an IL-10 polypeptide may be a nucleic acid molecule encoding any IL-10 polypeptide described herein or known in the art, including, for example, a nucleic acid molecule encoding a human IL-10 polypeptide, a nucleic acid molecule encoding a mouse IL-10 polypeptide, etc. Exemplary nucleic acid molecules encoding IL-10 polypeptides are provided as SEQ ID NOS: 16 and 17. Nucleic acids of the present disclosure that encode an IL-10 polypeptide also include nucleic acids having at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NOS: 16 or 17. In some embodiments, the nucleic acid comprises a sequence at least 90%, at least 91%, at least 92%, at least 93%, at least 94%, at least 95%, at least 96%, at least 97%, at least 98%, at least 99%, or 100% identical to the sequence of SEQ ID NO: 16.

In some embodiments, a nucleic acid encoding an IL-10 polypeptide is a nucleic acid that encodes an N-terminal truncation, a C-terminal truncation, or a fragment of any of the IL-10 polypeptides described herein or known in the art. For example, a nucleic acid encoding an N-terminal truncation, a C-terminal truncation, or a fragment of a human IL-10 polypeptide may be a nucleic acid having at least 25, at least 50, at least 75, at least 100, at least 150, at least 200, at least 250, at least 300, at least 350, at least 400, at least 450, at least 500, or at least 525, but fewer than 537, consecutive nucleotides of SEQ ID NO: 16. In some embodiments, a nucleic acid encoding a fragment of human IL-10 is a nucleic acid encoding positions 55-537 of SEQ ID NO: 16.

In some embodiments, the IL-10 polypeptide is any of the IL-10 polypeptides described herein or known in the art, including, for example, a human IL-10 polypeptide, a mouse IL-10 polypeptide, etc. Exemplary amino acid sequences of IL-10 polypeptides include the amino acid sequences of SEQ ID NOS: 29 and 30. In some embodiments, a nucleic acid encoding an IL-10 polypeptide is a nucleic acid that encodes a polypeptide having at least 75%, at least 80%, at least 85%, at least 90%, at least 91%, at least 92%, at least 93%, at least 94%, at least 95%, at least 96%, at least 97%, at least 98%, at least 99%, or 100% amino acid sequence identity to the sequence of SEQ ID NOS: 29 or 30. In some embodiments, the IL-10 polypeptide comprises a sequence at least 90%, at least 91%, at least 92%, at least 93%, at least 94%, at least 95%, at least 96%, at least 97%, at least 98%, at least 99%, or 100% identical to the amino acid sequence of SEQ ID NO: 29. In some embodiments, the present disclosure relates to polynucleotides that encode polypeptides that are homologs of the H. sapiens IL-10. Methods of identifying polypeptides that are homologs of a polypeptide of interest are well known to one of skill in the art.

In some embodiments, a nucleic acid encoding an IL-10 polypeptide is a nucleic acid that encodes N-terminal truncations. C-terminal truncations, or fragments of the amino acid sequence of SEQ ID NOS: 29 or 30. For example, an N-terminal truncation, C-terminal truncation, or fragment of a human IL-10 polypeptide may comprise at least 10, at least 12, at least 14, at least 16, at least 18, at least 20, at least 30, at least 40, at least 50, at least 75, at least 100, at least 125, at least 150, or at least 175, but fewer than 178, consecutive amino acids of SEQ ID NO: 29. In some embodiments, a fragment of human IL-10 is a polypeptide comprising positions 19-178 of SEQ ID NO: 29.

### Linker Sequence

In some embodiments, a polynucleotide of the present disclosure comprises a linker sequence between the CD11b promoter and the therapeutic polypeptide. In some embodiments, the linker sequence is a non-coding sequence lacking any functional features or characteristics. In some embodiments, the linker sequence maintains the open reading frame of the therapeutic polypeptide in-frame with the CD11b promoter. In some embodiments, the linker sequence comprises one or more expression control sequences (e.g., a sequence that contributes to the regulation of transcription and/or translation of the therapeutic polypeptide operably linked to the CD11b promoter).

In some embodiments, the linker sequence comprises a nucleic acid sequence having at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 31. In some embodiments, the linker sequence comprises the nucleic acid sequence of SEQ ID NO: 31. In some embodiments, the linker sequence comprises a 5′ truncation, a 3′ truncation, or a fragment of the sequence of SEQ ID NO: 31. For example, a linker sequence comprising a 5′ truncation, a 3′ truncation, or a fragment of the sequence of SEQ ID NO: 31 may include a nucleic acid molecule having at least 1, at least 2, at least 4, at least 6, at least 8, at least 10, at least 12, at least 14, at least 16, at least 18, at least 20, at least 25, at least 30, at least 35, or at least 40, but fewer than 41, consecutive nucleotides of SEQ ID NO: 31.

### Exemplary Polynucleotides

In some embodiments, a polynucleotide of the present disclosure comprises a nucleic acid sequence having at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 12. In some embodiments, the polynucleotide comprises the nucleic acid sequence of SEQ ID NO: 12. In some embodiments, the polynucleotide comprises a 5′ truncation, a 3′ truncation, or a fragment of the sequence of SEQ ID NO: 12. For example, a polynucleotide comprising a 5′ truncation, a 3′ truncation, or a fragment of the sequence of SEQ ID NO: 12 may include a nucleic acid molecule having at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or at least 1200, but fewer than 1203, consecutive nucleotides of SEQ ID NO: 12.

In some embodiments, a polynucleotide of the present disclosure comprises a nucleic acid sequence having at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 13. In some embodiments, the polynucleotide comprises the nucleic acid sequence of SEQ ID NO: 13. In some embodiments, the polynucleotide comprises a 5′ truncation, a 3′ truncation, or a fragment of the sequence of SEQ ID NO: 13. For example, a polynucleotide comprising a 5′ truncation, a 3′ truncation, or a fragment of the sequence of SEQ ID NO: 13 may include a nucleic acid molecule having at least 25, at least 50, at least 75, at least 100, at least 150, at least 200, at least 250, at least 300, at least 350, at least 400, at least 450, at least 5), at least 550, at least 600, at least 650, at least 700, at least 750, at least 800, at least 850, at least 900, at least 950, at least 1000, at least 1050, at least 1100, or at least 1150, but fewer than 1200, consecutive nucleotides of SEQ ID NO: 13.

### Vectors

In some aspects, the present disclosure relates to vectors, including expression vectors, containing one or more polynucleotides described herein. In some embodiments, the vectors are DNA vectors or RNA vectors. Generally, vectors suitable to maintain, propagate, or express polynucleotides to produce one or more polypeptides in a subject may be used. Examples of suitable vectors include, but are not limited to, plasmids, cosmids, episomes, transposons, and viral vectors (e.g., adenoviral, vaccinia viral, Sindbis-viral, measles, herpes viral, lentiviral, retroviral, adeno-associated viral vectors, etc.). In some embodiments, the vector is a viral vector (e.g., a replication defective viral vector). In some embodiments, the vector is an adenovirus vector or a lentivirus vector. In some embodiments, the vector is capable of autonomous replication in a host cell. In some embodiments, the vector is incapable of autonomous replication in a host cell. In some embodiments, the vector is capable of integrating into host DNA. Methods for making vectors containing one or more polynucleotides of interest are well known to one of ordinary skill in the art.

A vector may include a polynucleotide of the present disclosure in a form suitable for expression of the polynucleotide in a host cell. Expression vectors may include one or more regulatory sequences operatively linked to the polynucleotide to be expressed. The term “regulatory sequence” includes enhancers and other expression control elements (e.g., polyadenylation signals). Examples of suitable enhancers may include, but are not limited to, enhancer sequences from mammalian genes (such as globin, elastase, albumin, α-fetoprotein, insulin and the like), and enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like). Regulatory sequences may include those which direct constitutive expression of a nucleotide sequence, as well as tissue-specific regulatory and/or inducible sequences. The design of the vector may depend on such factors as the cell to be contacted with a polynucleotide of the present disclosure, the level of expression of therapeutic polypeptide desired, and the like. The expression vectors of the present disclosure may be introduced into host cells to thereby produce polypeptides encoded by polynucleotides as described herein.

Vectors of the present disclosure may further encode additional coding and non-coding sequences. Examples of additional coding and non-coding sequences may include, but are not limited to, sequences encoding additional polypeptide tags, introns, 5′ and 3′ UTRs, and the like. Examples of suitable polypeptide tags may include, but are not limited, to any combination of purification tags, such as his-tags, flag-tags, maltose binding protein and glutathione-S-transferase tags, detection tags, such as tags that may be detected photometrically (e.g., red fluorescent protein, etc.) and tags that have a detectable enzymatic activity (e.g., alkaline phosphatase, etc.), tags containing secretory sequences, leader sequences, and/or stabilizing sequences, protease cleavage sites (e.g., furin cleavage sites, TEV cleavage sites, Thrombin cleavage sites), and the like. In some embodiments, the 5′ and/or 3′UTRs increase the stability, localization, and/or translational efficiency of the polynucleotides. In some embodiments, the 5′ and/or 3′UTRs are modified to increase the stability, localization, and/or translational efficiency of the one or more polynucleotides. In some embodiments, the 5′ and/or 3′UTRs improve the level and/or duration of protein expression. A vector may also include one or more selectable marker genes and other genetic elements known in the art.

### IV. Cells

Certain aspects of the present disclosure relate to increasing expression of one or more therapeutic polypeptides in one or more cells (e.g., that naturally and/or endogenously express CD11b), such as one or more human cells, using any of the polynucleotides and/or vectors described herein.

As disclosed herein, one or more “human cell(s)” refers to any one or more cell(s) found throughout the human body after embryonic development, such as human stein cells, differentiated cells, mature cells, somatic cells, and adult cells. In some embodiments, human cells of the present disclosure are human adult cells. Human cells of the present disclosure include, without limitation, mature cells, differentiated cells, somatic cells, progenitor cells, induced pluripotent stem (iPS) cells, adult stein cells, somatic stein cells, and tissue stem cells. Adult stem cells, which are also known as somatic stem cells or tissue stem cells, may refer to undifferentiated cells, found throughout the body after embryonic development, which multiply by cell division to replenish dying cells and regenerate damaged tissues. Progenitor cells may refer to oligopotent or unipotent cells that differentiate into a specific type of cell or cell lineage. Progenitor cells are similar to stem cells but are more differentiated and exhibit limited self-renewal. Exemplary adult stem cells, tissue stem cells, and/or progenitor cells may include, without limitation, hematopoietic stem cells, mesenchymal stem cells, adipose stem cells, neuronal stem cells, intestinal stem cells, skin stem cells, etc. Human cells may also include, without limitation, somatic cells, mature cells, and differentiated cells. Somatic cells may refer to any cell of the body, including, without limitation, tissue stem cells, progenitor cells, induced pluripotent stein (iPS) cells, and differentiated cells. Exemplary somatic cells, mature cells, and/or differentiated cells may include, without limitation, epidermal cells, fibroblasts, lymphocytes, hepatocytes, epithelial cells, myocytes, chondrocytes, osteocytes, adipocytes, cardiomyocytes, pancreatic N cells, keratinocytes, erythrocytes, peripheral blood cells, bone marrow cells, neurocytes, astrocytes, etc.

In some embodiments, the one or more cells are a population of cells comprising one or more cells from the myeloid lineage. Examples of cells in the myeloid lineage may include any myeloid lineage cell described herein or known in the art, including, for example, hematopoietic stem cells, myeloid progenitor cells, megakaryocytes, thrombocytes, erythrocytes, mast cells, myeloblasts, basophils, neutrophils, eosinophils, monocytes, macrophages, and any combinations thereof. In some embodiments, the polynucleotides and/or vectors of the present disclosure are introduced directly into one or more myeloid lineage cells (e.g., as described below), and/or one or more cells in a population of cells are one or more progeny cells of a myeloid lineage cell (e.g., a hematopoietic stem cell) contacted with the polynucleotide and/or vector.

In some embodiments, the polynucleotide and/or vector is contacted with and/or introduced into a population of cells ex vivo. In some embodiments, the population of cells comprises primary cells isolated from a subject. In some embodiments, the population of cells comprises hematopoietic stein cells isolated from a subject. In some embodiments, the hematopoietic stem cells are isolated from a subject suffering from a disease or condition described herein. In some embodiments, the hematopoietic stem cells are isolated from a healthy donor (e.g., an allograft). In some embodiments, the healthy donor is a relative of a subject suffering from a disease or condition described herein. Methods of isolating hematopoietic stem cells from a patient are well known to one of ordinary skill in the art, including, for example, by apheresis.

### Expression of Therapeutic Polypeptides

In some embodiments, the polynucleotides and/or vectors of the present disclosure transiently increase expression of the therapeutic polypeptide in the contacted human cells. For example, a polynucleotide and/or vector of the present disclosure may increase expression of the therapeutic polypeptide for about 1 hour to about 23 hours (e.g., for about 1 hour, about 2 hours, about 3 hours, about 4 hours, about 5 hours, about 6 hours, about 7 hours, about 8 hours, about 9 hours, about 10 hours, 11 hours, about 12 hours, about 13 hours, about 14 hours, about 15 hours, about 16 hours, about 17 hours, about 18 hours, about 19 hours, about 20 hours, about 21 hours, about 22 hours, or about 23 hours): about 1 day to about 10 days (e.g., about 1 day, about 1.25 days, about 1.5 days, about 1.75 days, about 2 days, about 2.25 days, about 2.5 days, about 2.75 days, about 3 days, about 3.25 days, about 3.5 days, about 3.75 days, about 4 days, about 4.25 days, about 4.5 days, about 4.75 days, about 5 days, about 6.25 days, about 6.5 days, about 6.75 days, about 7 days, about 7.25 days, about 7.5 days, about 7.75 days, about 8 days, about 8.25 days, about 8.5 days, about 8.75 days, about 9 days, about 9.25 days, about 9.5 days, about 9.75 days, or about 10 days); about 1 week to about 10 weeks (e.g., about 1 week, about 2 weeks, about 3 weeks, about 4 weeks, about 5 weeks, about 6 weeks, about 7 weeks, about 8 weeks, about 9 weeks, or about 10 weeks); etc.

In some embodiments, the polynucleotides and/or vectors of the present disclosure increase expression of the therapeutic polypeptide in one or more contacted cells (e.g., in a macrophage at the site of inflammation). For example, a polynucleotide and/or vector of the present disclosure may increase expression of the therapeutic polypeptide in the one or more contacted cells by at least 1.5 fold, at least 2.0 fold, at least 3.0 fold, at least 4.0 fold, at least 5.0 fold, at least 6.0 fold, at least 7.0 fold, at least 8.0 fold, at least 9.0 fold, at least 10 fold, at least 100 fold, or at least 1,000 fold, relative to the therapeutic polypeptide expression in a corresponding cell that has not been contacted with the polynucleotide and/or vector. In some embodiments, the contacted cell does not naturally and/or endogenously express the therapeutic polypeptide, and the polynucleotide and/or vector of the present disclosure causes the cell to unnaturally and/or exogenously express the therapeutic polypeptide. In some embodiments, the polynucleotides and/or vectors of the present disclosure increase expression of the therapeutic polypeptide only when the cell comprising the polynucleotide and/or vector expresses endogenous CD11b (e.g., in a macrophage such as at the site of inflammation). Methods of measuring mRNA or protein expression of a target of interest are well known to one of ordinary skill in the art, including, for example, by ELISA (see e.g., Example 1 below).

Methods of Introducing Polynucleotide and/or Vectors

Introducing a polynucleotide and/or vector of the present disclosure into a cell encompasses any means of delivering a nucleic acid molecule known in the art. For example, nucleic acid molecules can be transfected, transduced or electroporated into a cell. Introduction of a polynucleotide of the present disclosure into human cells (e.g., hematopoietic stem cells) may involve using a viral vector (such as integrating or non-integrating viral vectors) or a plasmid vector, and/or delivery of mRNA molecules. Each of these methods has been described in the art and is therefore within the capabilities of one of skill in the art.

### V. Compositions

Certain aspects of the present disclosure relate to a composition comprising any of the polynucleotides, vectors, and or cells (e.g., a population of cells) described herein. In some embodiments, the composition is a pharmaceutical composition comprising any of the polynucleotides, vectors, and or cells (e.g., a population of cells) described herein and a pharmaceutically acceptable carrier. The composition may be prepared by conventional methods known in the art.

The term “pharmaceutically acceptable carrier” refers to any inactive substance that is suitable for use in a formulation for the delivery of a polynucleotide, vector, and/or cell. A carrier may be an anti-adherent, binder, coaling, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solutions, microemulsions, dispersions, or suspensions. Examples of solid dosage forms include tablet, pill, capsule, microcapsule, and powder. A particular form of the composition is a sterile liquid, such as a solution, suspension, or dispersion, for injection or infusion. Sterile solutions can be prepared by incorporating the polynucleotide and/or vector in the required amount in an appropriate carrier, followed by sterilization microfiltration. Generally, dispersions are prepared by incorporating the active ingredient into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

In addition to the polynucleotides, vectors, and/or cells, one or more additional therapeutic agents may be included in the composition. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent which produces a therapeutic effect, but may be a lesser amount as well. In some embodiments, the additional therapeutic agent is an immunosuppressant. Any immunosuppressant known in the art may be used, including, for example, glucocorticoids (such as prednisone, dexamethasone, hydrocortisone, etc.), cytostatics (such as purine analogs, folic acid analogs, pyrimidine analogs, protein synthesis inhibitors, cyclophosphamide, nitrosoureas, platinum compounds, methotrexate, azathioprine, mercaptopurine, dactinomycin, anthracyclines, mitomycin C, bleomycin, mithramycin, etc.), immunosuppressive antibodies, drugs acting on immunophilins (such as ciclosporin, tacrolimus, sirolimus, everolimus, etc.), and others (such as interferons, opioids. TNF binding proteins, mycophenolate, fingolimod, myriocin, etc.). In some embodiments, the immunosuppressant is prednisone, deflazacort, cytotoxic T-lymphocyte-associated protein-4, and/or any combinations thereof.

Any of the polynucleotides, vectors, cells, and/or compositions described herein may be used in the preparation of a medicament (e.g., a medicament for use in treating or delaying progression of muscular dystrophy, polymyositis, dermatomyositis, multiple sclerosis, and/or autoimmune demyelination in a subject in need thereof).

### VI. Methods of Treatment

Certain aspects of the present disclosure relate to the use of any of the polynucleotides, vectors, cells, and/or compositions described herein for the treatment of a subject. In some embodiments, the subject is a human. In some embodiments, the subject suffers from a disease or condition (e.g., muscular dystrophy, polymyositis, dermatomyositis, multiple sclerosis, and autoimmune demyelination). In some embodiments, the methods of the present disclosure include the steps of: (a) isolating cells (e.g., one or more hematopoietic cells) from an individual (e.g., the subject, a healthy donor). (b) contacting the cells with a polynucleotide, vector, and/or composition described herein, and (c) administering the contacted cells to the subject.

In some embodiments, use of the methods of the present disclosure reduces one or more signs or symptoms of a disease or condition in a subject, and/or treats one or more of the underlying causes of the disease or condition in the subject. In some embodiments, use of the methods of the present disclosure allows for the selective expression of one or more therapeutic polypeptides in cells that naturally and/or endogenously express CD11b (e.g., myeloid lineage cells such as macrophages) in the subject. In some embodiments, use of the methods of the present disclosure allows for the selective expression of one or more therapeutic polypeptides in particular contexts (e.g., in activated macrophages at sites of inflammation) in the subject. In some embodiments, use of the methods of the present disclosure allows for the delivery of therapeutic polypeptides directly to the site of disease (e.g., using the (inflammatory) cell migration of cells comprising a polynucleotide described herein), without dangerous, off-target effects in other, non-diseased tissues. In some embodiments, use of the methods of the present disclosure reduces inflammation in the subject (e.g., inflammation at a particular site or tissue within the subject). In some embodiments, use of the methods of the present disclosure reduces fibrosis in the subject.

In some embodiments, methods of the present disclosure include administering an effective amount of a population of cells comprising one or more cells comprising any of the polynucleotides and/or vectors described herein. In some embodiments, the population of cells comprises hematopoietic stem cells previously contacted with any of the polynucleotides and/or vectors described herein (e.g., previously contacted ex vivo). In some embodiments, the hematopoietic stem cells were isolated from the subject. In some embodiments, the hematopoietic stem cells were isolated from a healthy donor (e.g., a relative of the subject). In some embodiments, the population of cells comprises one or more myeloid lineage cells differentiated from a hematopoietic stem cell contacted with any of the polynucleotides and/or vectors described herein. In some embodiments, the one or more myeloid lineage cells are one or more of megakaryocytes, thrombocytes, erythrocytes, mast cells, myeloblasts, basophils, neutrophils, eosinophils, monocytes, macrophages, and any combinations thereof. In some embodiments, one or more cells (e.g., one or more myeloid lineage cells) from the population of cells localizes to a site of inflammation in the subject. In some embodiments, the one or more myeloid lineage cells expresses the therapeutic polypeptide from the CD11b promoter when the one or more myeloid lineage cells naturally expresses endogenous CD11b (e.g., in activated macrophages at a site of inflammation).

In some embodiments, the subject suffers from muscular dystrophy, and administration of any of the polynucleotides, vectors, cells, and/or compositions described herein reduces one or more signs, symptoms, or complications associated with muscular dystrophy. Signs, symptoms, or complications associated with muscular dystrophy may include, without limitation, progressive muscle weakness, frequent falls, difficulty rising from a lying or sitting up position, trouble walking, running and jumping, waddling gate, walking on the toes, large calf muscles, muscle pain and stiffness, inability to relax muscles at will following contractions (including in the face and neck), shortening of muscles or tendons around the joints (contractures), breathing problems, scoliosis, heart problems, swallowing problems, and any combinations thereof.

In some embodiments, the subject suffers from polymyositis, and administration of any of the polynucleotides, vectors, cells, and/or compositions described herein reduces one or more signs, symptoms, or complications associated with polymyositis. Signs, symptoms, or complications associated with polymyositis may include, without limitation, muscle weakness (including the muscles closest to the trunk, such as the hips, thighs, shoulders, upper arms, and neck), difficulty swallowing, aspiration pneumonia, breathing problems, complications with other conditions often associated polymyositis (such as Raynaud's phenomenon, connective tissue diseases, cardiovascular disease, lung disease, cancer, etc.), and any combinations thereof.

In some embodiments, the subject suffers from dermatomyositis, and administration of any of the polynucleotides, vectors, cells, and/or compositions described herein reduces one or more signs, symptoms, or complications associated with dermatomyositis. Signs, symptoms, or complications associated with dermatomyositis may include, without limitation, skin changes (such as the development of a violet-colored or dusky red rash (commonly on the face, eyelids, knuckles, elbows, knees, chest, and/or back)), muscle weakness, difficulty swallowing, aspiration pneumonia, breathing problems, calcium deposits, and any combinations thereof.

In some embodiments, the subject suffers from multiple sclerosis, and administration of any of the polynucleotides, vectors, cells, and/or compositions described herein reduces one or more signs, symptoms, or complications associated with multiple sclerosis. Signs, symptoms, or complications associated with multiple sclerosis may include, without limitation, numbness or weakness in one or more limbs, partial or complete loss of vision, prolonged double vision, tingling or pain in parts of the body, electric-shock sensations that occur with certain neck movements (such as bending the neck forward), tremors, lack of coordination or unsteady gait, slurred speech, fatigue, dizziness, problems with bowel and bladder function, muscle stiffness or spasms, paralysis (typically in the legs), mental changes (such as forgetfulness or mood swing), depression, epilepsy, and any combinations thereof.

In some embodiments, the subject suffers from autoimmune demyelination, and administration of any of the polynucleotides, vectors, cells, and/or compositions described herein reduces one or more signs, symptoms, or complications associated with autoimmune demyelination. Signs, symptoms, or complications associated with autoimmune demyelination may include, without limitation, blurred double vision, ataxia, clonus, dysarthria, fatigue, clumsiness, hand paralysis, hemiparesis, genital anesthesia, incoordination, parasthesias, ocular paralysis, impaired muscle coordination, muscle weakness, low of sensation, unsteady gait, spastic paraparesis, incontinence, hearing problems, speech problems, and any combinations thereof.

In practicing the methods of the present disclosure, the polynucleotides, vectors, cells, and/or compositions may be administered alone as a monotherapy, or administered in combination with one or more additional therapeutic agents. Thus, in another aspect, the present disclosure provides a combination therapy with one or more additional therapeutic agents for separate, sequential, or simultaneous administration. In some embodiments, the one or more additional therapeutic agents are one or more immunosuppressants described herein.

The polynucleotides, vectors, cells, and/or compositions provided herein may be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The suitable route and method of administration may vary depending on a number of factors such as the therapeutic being used, the rate of absorption desired, specific formulation or dosage form used, type or severity of the disorder being treated, the specific site of action, and conditions of the patient, and can be readily selected by a person skilled in the art.

A polynucleotide, vector, cell, and/or composition may be administered once or on multiple occasions. Intervals between single doses can be, for example, daily, weekly, monthly, every three months or yearly. An exemplary treatment regimen entails administration once per week, once every two weeks, once every three weeks, once every four weeks, once a month, once every three months or once every three to six months. Appropriate dosages and dosage schedules may be determined by a person skilled in the art.

### VII. Kits

Certain aspects of the present disclosure relate to an article of manufacture or kit comprising a polynucleotide, vector, cell, and/or composition described herein. In some embodiments, the kit further comprises a packed insert comprising instructions for the use of the polynucleotide, vector, cell, and/or composition. In some embodiments, the article of manufacture or kit further comprises one or more buffer, e.g., for storing, transferring, administering, or otherwise using the polynucleotide, vector, cell, and/or composition. In some embodiments, the kit further comprises one or more containers for storing the polynucleotide, vector, cell, and/or composition.

The foregoing written description is considered to be sufficient to enable one skilled in the art to practice the present disclosure. The following Examples are offered for illustrative purposes only, and are not intended to limit the scope of the present disclosure in any way. Indeed, various modifications of the present disclosure in addition to those shown and described herein will become apparent to those skilled in the art from the foregoing description and fall within the scope of the appended claims.

### EXAMPLES

### Example 1: Generation of a CD11b-Driven LIF Expression Cassette

Although the CD11b promoter has been used to drive the expression of non-therapeutic reporter genes in transgenic mice, and used for protein expression in lenti-virally transduced bone marrow cells (Dziennes et al. 1995; He et al. 2006), the CD11b promoter has not been used to drive expression of the therapeutic protein LIF using an endogenous system in which LIF is administered directly at the site of disease.

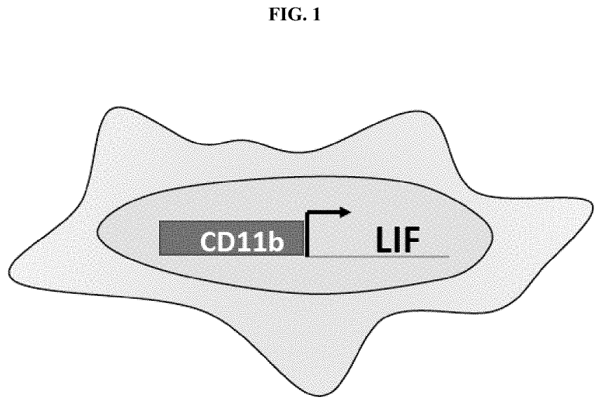

A hybrid DNA molecule was created that included two uniquely paired DNA sequences: 1) the CD11b promoter sequence that drives expression of a therapeutic molecule specifically in myeloid cells (immune cells of interest that naturally target diseased tissue), and 2) the cDNA sequence of the therapeutic molecule Leukemia Inhibitory Factor (LIF), which can mitigate inflammation and promote tissue growth and repair (FIG. 1). The LIF cDNA sequence was generated by reverse transcription of mouse mRNA using PFU for increased fidelity, and subsequent amplification using the TOP pR2 system. After sequencing, the verified LIF cDNA was subcloned to amplify. A transgenic mouse expressing the CD11b/LIF construct was then generated via microinjection into B6SJLF1/J eggs.

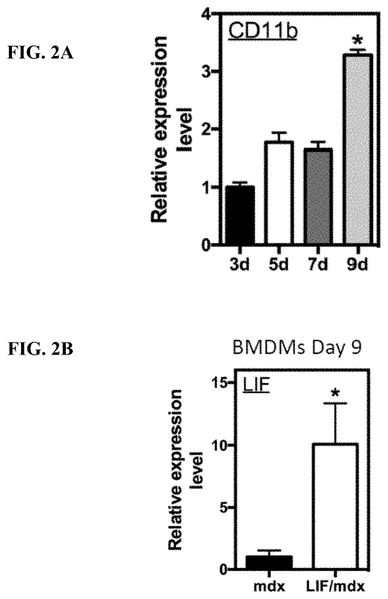

Efficacy of the CD11b/LIF construct was next tested in vitro and in vivo. Activated macrophages were observed to express high levels of endogenous CD11b, with an approximate 3-fold increase in endogenous CD11b expression at day 9 relative to day 3 (FIG. 2A). In agreement with the expression of endogenous CD11b, bone marrow-derived macrophages (BMDMs) transfected with the CD11b/LIF construct showed high LIF expression at day 9 in culture medium when activated, as measured by qPCR (FIG. 2B).

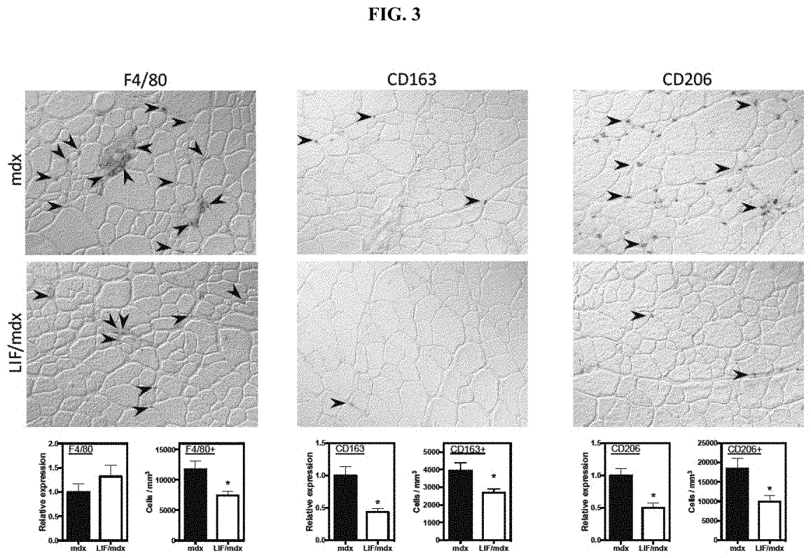

A dystrophic mouse model (mdx) was used to generate transgenic dystrophic mice carrying the CD11b/LIF construct (LIF/mdx). The numbers and distribution of macrophages in muscles from these two mouse lines were next tested. It was observed that dystrophic mice carrying the CD11b/LIF construct had reduced numbers of F4/80+. CD163+ and CD206+ macrophages in muscles from the LIF/mdx mice as compared to the control mdx mice (FIG. 3).

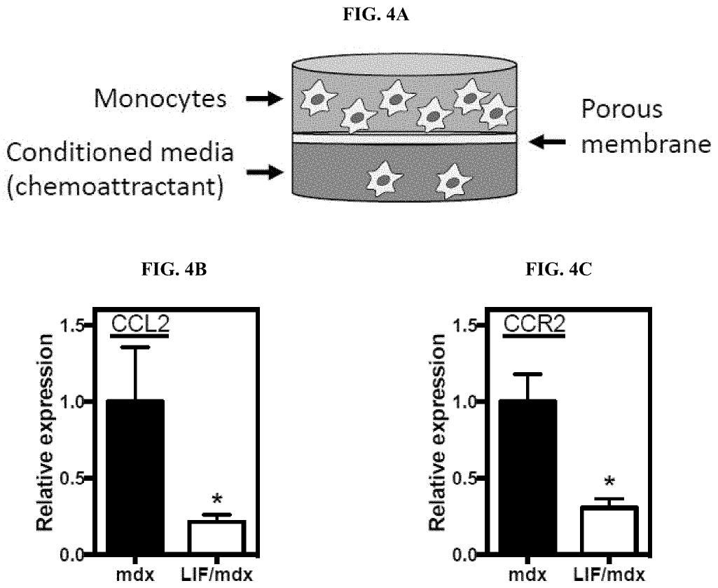

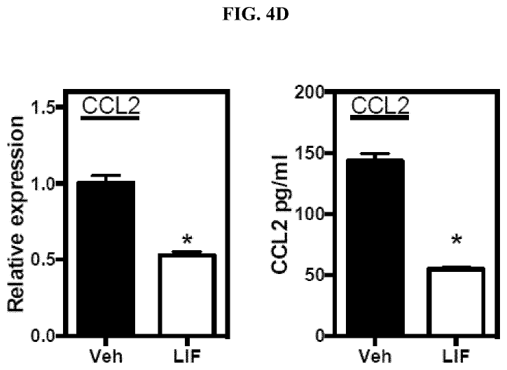

Next, the levels of two chemoattractants (CCL2 and CCR2) for inflammatory cells in muscle samples from the mdx and LIF/mdx mice were examined. The levels of CCL2 (FIG. 4B) and CCR2 (FIG. 4C) were both significantly reduced in muscle sample obtained from the mdx/LIF mice as compared to the control mdx dystrophic mice. The relative expression of CCL2 in untreated macrophages was compared to CCL2 levels expressed in macrophages treated with LIF. Interestingly, treatment of macrophages with LIF reduced the levels of CCL2 expression (FIG. 4D).

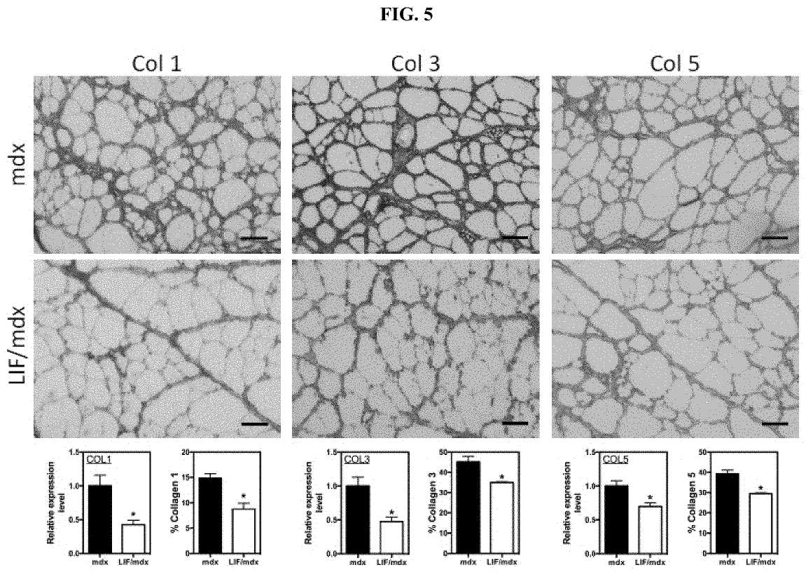

The numbers and distribution of multiple types of collagen (Col 1, Col 3, and Col 5) in dystrophic muscle from control mdx mice and the transgenic LIF/mdx mice was next tested. It was found that expression and accumulation of each of these types of collagen were reduced in muscle from the LIF/mdx mice as compared to the control mdx mice (FIG. 5).

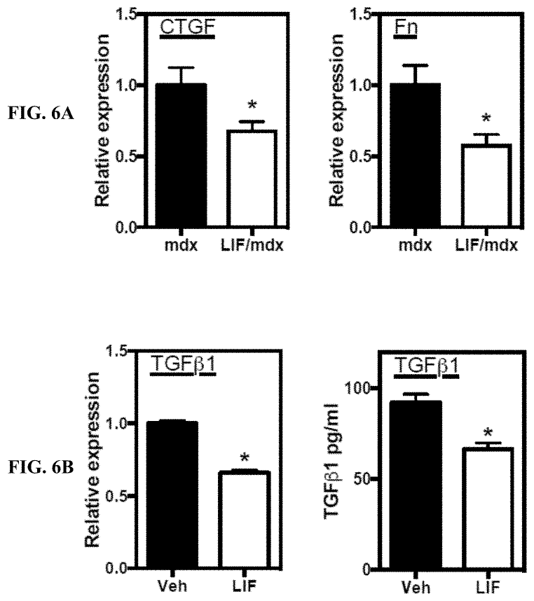

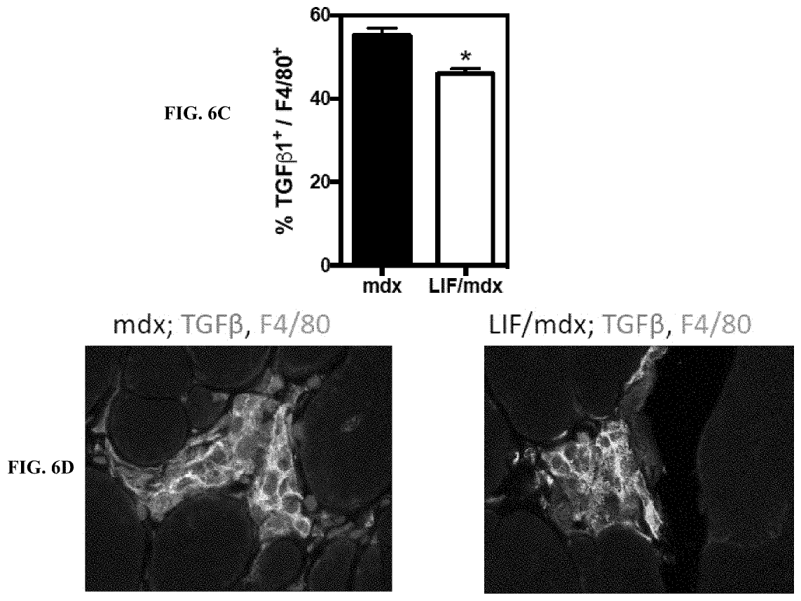

It was also found that the expression of connective tissue growth factor (CTGF) and fibronectin (Fn) in muscles of the dystrophic mdx mice was significantly higher than that of the LF/mdx transgenic mice (FIG. 6A). Expression of transforming growth factor beta (TGFβ) was also observed to be reduced in cells stimulated with LIF as compared to control cells (vehicle). Finally, the proportion of F4/80+ macrophages that express TGFβ was examined in muscles from the LIF/mdx and mdx mice. A smaller proportion of TGFβ⁺/F4/50⁺ macrophages were observed in muscles from the LIF/mdx mice as compared to control mdx mice (FIGS. 6C-6D).

Taken together, this data indicated that the transgenic mice expressing the CD11b/LIF construct showed increased LIF expression in myeloid cells, and ameliorative differences in damaged tissue compared to mice not expressing the CD11b/LIF construct. The data presented herein showed that: 1) selective LIF transgene expression reduced muscle inflammation and fibrosis; 2) bone marrow-derived immune cells could be used for the delivery of gene therapy to dystrophic muscle; 3) utilizing a selective promoter (CD11b) could minimize negative, off-target effects: 4) LIF reduced macrophages in dystrophic muscle: 5) reduction in macrophages may have been due to reduced CCL2; 6) decreased inflammation was associated with reduced fibrosis; and 7) LIF inhibited macrophage expression of pro-fibrotic cytokine TGFβ1.

### Example 2: Targeting a Therapeutic LIF Transgene to Muscle Via the Immune System Ameliorates Muscular Dystrophy

Many potentially-therapeutic molecules have been identified for treating Duchenne muscular dystrophy. However, targeting those molecules only to sites of active pathology is an obstacle to their clinical use. Because dystrophic muscles become extensively inflamed, it was tested whether expressing a therapeutic transgene in leukocyte progenitors that invade muscle would provide selective, timely delivery to diseased muscle. A transgene was designed in which leukemia inhibitory factor (LIF) is under control of a leukocyte-specific promoter and transplanted transgenic cells into dystrophic mice. Transplantation diminishes pathology, reduces Th2 cytokines in muscle and biases macrophages away from a CD163+/CD206+ phenotype that promotes fibrosis. Transgenic cells also abrogates TGFβ signaling, reduces fibro/adipogenic progenitor cells and reduces fibrogenesis of muscle cells. These findings indicate that leukocytes expressing a LIF transgene reduce fibrosis by suppressing type 2 immunity and highlight a novel application by which immune cells can be genetically modified as potential therapeutics to treat muscle disease.

### Introduction

Over recent years, investigators have identified numerous, potentially-therapeutic molecules for the treatment of Duchenne muscular dystrophy (DMD), a lethal and incurable muscle-wasting disease. For example, systemic delivery of therapeutic agents that can inhibit fibrosis (e.g., block TGFβ function¹⁻³), inhibit muscle wasting (e.g., myostatin blocking molecules⁴), and increase numbers of muscle stem cells called satellite cells (e.g. Klotho⁵) all reduce pathology in the mdx mouse model of DMD. However, systemic delivery of any of these molecules presents risks of unintended off-target effects which provide an obstacle to their clinical application for the treatment of DMD. In addition, the occurrence of muscle pathology is not synchronized in DMD patients. The unpredictable timing and severity of disease vary between muscles in a single individual at any given time, and also vary between locations in a single muscle⁶. Even if a therapeutic agent were specifically targeted to dystrophic muscle, achieving delivery only when pathology is active presents an additional challenge.

Nature has provided a naturally-occurring system for targeted delivery of potentially-therapeutic molecules to dystrophic muscle at stages of the disease when pathology is active. Coinciding with the unpredictable ebb and flow of pathology in muscular dystrophy, inflammatory cells invade in numbers that coincide with the magnitude of muscle pathology. Although the immune cell infiltrate in dystrophin-deficient muscle is complex⁷⁻¹², macrophages comprise the vast majority and they can reach concentrations that exceed 10⁷ cells per pound of muscle at the peak of mdx pathology⁷. They are also rich sources of regulatory molecules that can amplify muscle damage but also promote muscle repair and regeneration in muscular dystrophy^{7,13,14}. Thus, introduction of therapeutic transgenes that and expressed at elevated levels in activated macrophages or other immune cells could provide a strategy for regulated targeting (facilitated with the assistance of endogenous systems) of therapeutic molecules specifically to dystrophic muscles at the time of active pathology and at levels that were commensurate with the extent of pathology.

In this investigation, it was tested whether transplantation of bone marrow cells (BMCs) into which a leukemia inhibitory factor (LIF) transgene controlled by the human CD11b promoter is introduced reduces the pathology of mdx dystrophy. Although mdx pathology is less severe than DMD pathology, they share the pathological features of muscle inflammation and progressive fibrosis that persist over the entire lifespan and impair muscle function, reduce health and increase mortality. The CD11b promoter was chosen to drive the therapeutic transgene because CD11b is expressed at low or undetectable levels in myeloid precursors, but at increasingly elevated levels during myeloid cell differentiation and activation¹⁵⁻¹⁷. LIF was selected as a therapeutic molecule to test this system because it is expressed by macrophages and can influence muscle growth, fibrosis and inflammation during disease or following injury¹⁸⁻²¹. The findings show that this intervention significantly modifies intramuscular macrophage phenotype and reduces inflammation and fibrosis of dystrophic muscle, thereby reducing pathology. Perhaps more valuable, the findings indicate that inflammatory cells can be exploited as vectors to deliver therapeutic transgenes for the treatment of chronic diseases in which there is a significant inflammatory component.

### Materials and Methods

Mice

All experimentation complied with all relevant ethical regulations for animal testing and research, and the study protocol was approved by the Chancellor's Animal Research Committee at the University of California, Los Angeles. C57BL/10ScSn-Dmd^{mdx}/J mice (mdx mice) were purchased from The Jackson Laboratory (Bar Harbor, Me.) and bred in pathogen-free vivaria. Mdx mice were selected for use in these experiments instead of more rapidly progressive models of DMD because the goal of the investigation is to test the hypothesis that transplantation of genetically-modified bone marrow cells provides a novel therapeutic strategy for muscular dystrophy. If a rapidly, progressive mouse model, such as the mdx/utr-mouse line in which the mice die at 2 to 3 months of age, was used, one would be unable to assay for treatment effects achieved by bone marrow transplantation because the mice would die before enough time passed for sufficient BMC engraftment and then for the transplanted cells to mediate their therapeutic effects.

In preparation for generating CD11b/LIF mice, the complete Mus musculus LIF cDNA sequence (611-bp; NM_008501) was amplified by PCR and ligated into a pGL3-Basic vector (Promega) at the NcoI/XbaI sites. The pGL3-Basic vector contained a 550-bp fragment of the human CD11b promoter at the Hind III site, upstream of the LIF insertion site. The 1215-bp, hCD11b/LIF fragment was isolated from pGL3-Basic by restriction endonuclease digestion with XhoI/XbaI and used for pronuclear injection into CB6F1 eggs to generate transgenic mice. Positive founders were identified by PCR screening for presence of the hCD11b/LIF construct and backcrossed with C57BL/6J mice for 7 generations. The hCD11b/LIF line is maintained as hemizygous to produce transgenic mice and wild-type, littermate controls for experimentation. Mice were randomly allocated to experimental groups. WT or CD11b/LIF BMCs were transplanted into mdx mice assigned non-sequential identification numbers. Investigators collecting data and performing analysis were aware of animal numbers only and were blinded to treatment groups.

CD11b/LIF mdx transgenic mice were produced by crossing CD11b/LIF hemizygous males with mdx females to generate CD11b/LIF hemizygous, transgenic mice that were also dystrophin-deficient (CD11b/LIF mdx). Dystrophin-deficient status was verified by ARMS PCR screening⁶⁵ and presence of the hCD11b/LIF construct was determined as described above. The CD11b/LIF mdx mice were backcrossed with wild-type mdx mice for 7 generations to produce CD11b/LIF mdx mice that were dystrophin-deficient and either hemizygous or wild-type controls for the CD11b/LIF transgene.

Bone Marrow Transplantation

Beginning one week prior to BMT, mouse drinking water was supplemented with trimethoprim/sulfamethoxazole (80 μg/ml trimethoprim and 400 sg/ml sulfamethoxazole) and continued for 3-weeks. Two-month-old female mdx mice underwent myeloablative preconditioning via intraperitoneal injections of 1,4-butanediol dimethanesulfonate (Sigma-Aldrich) (20 mg/kg body weight) 72-, 48- and 24-hours prior to BMT. On the day of transplantation, male WT and CD11b/LIF donor mice were euthanized and their femur and tibia bones were sterilely dissected and flushed of BMCs. BMCs were isolated and recipient mice received 10⁷ donor BMCs by tail-vein injection. At 4-months post-BMT, tissues and BMCs were collected from recipient mice. BMCs were used for chimerism analysis by fluorescent in situ hybridization of the Y-chromosome (Kreatech FISH Probes).

RNA Isolation and QPCR

RNA was isolated from muscle homogenates and reverse transcribed to produce cDNA²⁴. QPCR experiments were designed using established guidelines for experimental design, data normalization and data analysis to maximize the rigor of quantifying the relative levels of mRNA^{13,66,67}. The expression for each gene in control samples was set to 1 and the other expression values were then scaled to that value. PCR primers are listed in Table 1.

TABLE 1

| Gene | Forward | Reverse |
|---|---|---|
| CdI1b | CATGAATGATGCT | CCCAAAATAAGAG |
| | TACCTGGGTTATG | CCAATCTGG |
| | (SEQ ID NO: 32) | (SEQ ID NO: 67) |
| Lif | GTCTTGGCCGCAG | CCCAAAATAAGAG |
| | GGATTG | CCAATCTGG |
| | (SEQ ID NO: 33) | (SEQ ID NO: 68) |
| Serpinh1 | GACCCATGACCTG | GAAGGCAGTGGCA |
| | CAGAAAC | TGGAAC |
| | (SEQ ID NO: 34) | (SEQ ID NO: 69) |
| Cd68 | CAAAGCTTCTGCT | GACTGGTCACGGT |
| | GTGGAAAT | TGCAAG |
| | (SEQ ID NO: 35) | (SEQ ID NO: 70) |
| iNOS | CAGCACAGGAAAT | TAGCCAGCGTACC |
| | GTTTCAGC | GGATGA |
| | (SEQ ID NO: 36) | (SEQ ID NO: 71) |
| Cd163 | GCAAAAACTGGCA | GTCAAAATCACAG |
| | GTGGG | ACGGAG |
| | (SEQ ID NO: 37) | (SEQ ID NO: 72) |
| Cd206 | GGATTGTGGAGCA | CTTGAATGGAAAT |
| | GATGGAAG | GCACAGAC |
| | (SEQ ID NO: 38) | (SEQ ID NO: 73) |
| Arg1 | CAATGAAGAGCTG | GTGTGAGCATCCA |
| | GCTGGTGT | CCCAAATG |
| | (SEQ ID NO: 39) | (SEQ ID NO: 74) |
| Arg2 | GAAGTGGTTAGTA | GGTGAGAGGTGTA |
| | GAGCTGTGTC | TTAATGTCCG |
| | (SEQ ID NO: 40) | (SEQ ID NO: 75) |
| Tnf | CTTCTGTCTACTG | CACTTGGTGGTTT |
| | AACTTCGGG | GCTACGAC |
| | (SEQ ID NO: 41) | (SEQ ID NO: 76) |
| Ifng | GACAATCAGGCCA | CGGATGAGCTCAT |
| | TCAGCAAC | TGAATGCTT |
| | (SEQ ID NO: 42) | (SEQ ID NO: 77) |
| Il1b | GTAATGAAAGACG | CTCTGCAGACTCA |
| | GCACACC | AACTCC |
| | (SEQ ID NO: 43) | (SEQ ID NO: 78) |
| Il6 | GAACAACGATGAT | CTTCATGTACTCC |
| | GCACTTGC | AGGTAGCTATGGT |
| | (SEQ ID NO: 44) | (SEQ ID NO: 79) |
| Il12a | TGCCTTGGTAGCA | TTCAGGCGGAGCT |
| | TCTATGAG | CAGATAG |
| | (SEQ ID NO: 45) | (SEQ ID NO: 80) |
| Il4 | GGATGTGCCAAAC | GAGTTCTTCTTCA |
| | GTCCTC | AGCATGGAG |
| | (SEQ ID NO: 46) | (SEQ ID NO: 81) |
| Il10 | CAAGGAGCATTTG | GGCCTTGTAGACA |
| | AATTCCC | CCTTGGTC |
| | (SEQ ID NO: 47) | (SEQ ID NO: 82) |
| Tgfb1 | CTCCACCTGCAAG | CTTAGTTTGGACA |
| | ACCAT | GGATCTGG |
| | (SEQ ID NO: 48) | (SEQ ID NO: 83) |
| Socs3 | CTTTCTTATCCGC | CACTGGATGCGTA |
| | GACAGCTC | GGTTCTTG |
| | (SEQ ID NO: 49) | (SEQ ID NO: 84) |
| Ccr2 | CCTGTAAATGCCA | GTATGCCGTGGAT |
| | TGCAAGTTC | GAACTGAG |
| | (SEQ ID NO: 50) | (SEQ ID NO: 85) |
| Ccl2 | GCTCAGCCAGATG | CTCTCTCTTGAGC |
| | CAGTTAAC | TTGGTGAC |
| | (SEQ ID NO: 51) | (SEQ ID NO: 86) |
| Ccl7 | CAACCAGATGGGC | GATAACAGCTTCC |
| | CCAATG | CAGGGACAC |
| | (SEQ ID NO: 52) | (SEQ ID NO: 87) |
| Ccl8 | GATAAGGCTCCAG | CCCTGCTTGGTCT |
| | TCACCTGC | GGAAAAC |
| | (SEQ ID NO: 53) | (SEQ ID NO: 88) |
| Ccl12 | CTGGACCAGATGC | AAGATCACAGCTT |
| | GGTGAG | CCCGGG |
| | (SEQ ID NO: 54) | (SEQ ID NO: 89) |
| Col1a1 | TGTGTGCGATGAC | GGGTCCCTCGACT |
| | GTGCAAT | CCTACA |
| | (SEQ ID NO: 55) | (SEQ ID NO: 90) |
| Col3a1 | ATCCCATTTGGAG | GGACATGATTCAC |
| | AATGTTGTGC | AGATTCCAGG |
| | (SEQ ID NO: 56) | (SEQ ID NO: 91) |
| Col5a3 | CGGGGTACTCCTG | GCATCCCTACTTC |
| | GTCCTAC | CCCCTTG |
| | (SEQ ID NO: 57) | (SEQ ID NO: 92) |
| Axin2 | GACGCACTGACCG | CTGCGATGCATCT |
| | ACGATTC | CTCTCTGG |
| | (SEQ ID NO: 58) | (SEQ ID NO: 93) |
| Ctgf | GGACACCTAAAAT | GGCACAGGTCTTG |
| | CGCCAAGC | ATGAACATC |
| | (SEQ ID NO: 59) | (SEQ ID NO: 94) |
| Fn | GCTCAGCAAATCG | CTAGGTAGGTCCG |
| | TGCAGC | TTCCCACTG |
| | (SEQ ID NO: 60) | (SEQ ID NO: 95) |
| Snai1 | CTTGTGTCTGCAC | GTCAGCAAAAGCA |
| | GACCTGTG | CGGTTG |
| | (SEQ ID NO: 61) | (SEQ ID NO: 96) |
| Tpt1 | GGAGGGCAAGATG | CGGTGACTACTGT |
| | GTCAGTAG | GCTTTCG |
| | (SEQ ID NO: 62) | (SEQ ID NO: 97) |
| Rnps1 | AGGCTCACCAGGA | CTTGGCCATCAAT |
| | ATGTGAC | TTGTCCT |
| | (SEQ ID NO: 63) | (SEQ ID NO: 98) |
| Rpl13a | CCTGCTGCTCTCA | CGATAGTGCATCT |
| | AGGTTGTT | TGGCCTTT |
| | (SEQ ID NO: 64) | (SEQ ID NO: 99) |
| Srpl4 | AGAGCGAGCAGTT | CGGTGCTGATCTT |
| | CCTGAC | CCTTTTC |
| | (SEQ ID NO: 65) | (SEQ ID NO: 100) |
| Rplp0 | GGACCCGAGAAGA | GCTGCCGTTGTCA |
| | CCTCCTT | AACACC |
| | (SEQ ID NO: 66) | (SEQ ID NO: 101) |

Cultured cells were washed twice with ice-cold DPBS and collected in Trizol (Invitrogen). RNA was extracted and isolated with chloroform extraction and isopropyl alcohol precipitation, followed by clean-up with an RNA Clean and Concentrator Kit (Zymo Research). Total RNA was quantified, reverse transcribed and used for QPCR¹³.

RNA was isolated from FACS sorted cells by first sorting cells directly into Buffer RLT RNA lysis buffer (Qiagen). RNA was isolated using a Qiagen RNeasy Micro Kit according to the manufacturer's protocol. RNA yield was quantified using a BioDrop μLite. RNA (50 ng/reaction) was reverse transcribed using a qScript XLT cDNA Supermix (QuantaBio). QPCR experiments were performed on a QuantStudio 5 Real-Time PCR System (Thermo Fisher) with PerfeCTa SYBR Green Supermix, Low Rox (QuantaBio)¹³.

Immunohistochemistry

Muscles dissected from euthanized mice were frozen in liquid nitrogen-cooled isopentane. Cross-sections 10-μm thick were taken from the mid-belly of muscles and fixed in ice-cold acetone or 2% paraformaldehyde for 10 minutes. Endogenous peroxidase activity in the sections was quenched by immersion in 0.3% H₂O₂. Most sections were blocked for 1 hour with blocking buffer (3% bovine serum albumin (BSA), 2% gelatin and 0.05% Tween-20 in 50 mM Tris-HCl pH 7.6 containing 150 mM NaCl). Alternatively, sections were incubated with 10% horse serum in PBS with 0.1% Tween-20 or mouse-on-mouse blocking reagent (M.O.M. kit; Vector) for sections to be incubated with primary antibodies from goat or mouse origin, respectively. Sections were then incubated with: rat anti-mouse F4/80 (1:100, overnight at 4° C., eBioscience, clone BM8), rat anti-mouse CD68 (1:100, 3 hours at room temperature (RT), AbD Serotec, clone FA-11), rabbit anti-mouse CD163 (1:100, 3 hours at RT, Santa Cruz Biotech, clone M-96), rat anti-mouse CD206 (1:50.3 hours at RT, AbD Serotec, clone MR5D3), rat anti-CD4 (1:25, overnight at 4′C, Biolegend, clone GK1.5), rat anti-Ly-6B.2 (1:25, 2 hours at RT, Bio-Rad, clone 7/4), rabbit anti-collagen type 1 (1:50, 3 hours at RT, Chemicon, #AB745), goat anti-collagen type 3 (1:50, 3 hours at RT, Southern Biotech #1330-01), goat anti-collagen type 5 (1:50, overnight at 4° C., Southern Biotech, #1350-01), goat anti-LIF (1:66, overnight at 4′C. R&D systems, #AB-449), and mouse anti-developmental myosin heavy chain (1:100, overnight at 4° C., Novacastra, #106304). The sections were washed with phosphate buffered saline (PBS) and probed with biotin-conjugated secondary antibodies (1:200, 30 minutes at RT, Vector Laboratories). Sections were then washed with PBS and incubated with avidin D-conjugated HRP (1:1000, 30 minutes at RT, Vector). Staining was visualized with the peroxidase substrate, 3-amino-9-ethylcarbazole (Vector).

Immunofluorescence

For co-labeling of macrophages, sections were fixed in ice-cold acetone for 10 minutes and then incubated in blocking buffer for 1 hour. Sections were then incubated with rat anti-F4/80 and goat anti-CCL2 (1:50, R&D Systems, AB-479-NA) or rabbit anti-TGFβ1 (1:100, Abcam. #ab92486) overnight at 4′C. Sections were washed with PBS and then incubated with donkey anti-rat Dylight 488 (1:200, 30 min at RT, Abcam, #ab102260) and horse anti-rabbit IgG Dylight 594 (1:200, 30 min at RT, Vector, #DI-1094) or horse anti-goat IgG Dylight 594 (1:200, 30 min at RT, Vector. #DI-3094). Sections were then washed with PBS and mounted with Prolong Gold mounting media (Invitrogen).

For identification of CCR2+ macrophages, sections were fixed with 4% PFA for 10 minutes and then incubated with blocking buffer for 1 hour. Sections were then labeled with rabbit anti-mouse CCR2 (1:50, Abeam, clone E68) and rat anti-mouse CD68 or rat anti-mouse CD206 at 4° C., overnight. Sections were washed with PBS and then incubated with donkey anti-rat IgG Dylight 594 (1:200, 30 min at RT, Abcam, #ab102262) and horse anti-rabbit IgG Dylight 488 (1:200, 30 min at RT. Vector, #DI-1088).

For identification of fibrogenic satellite cells, sections were fixed in 2% paraformaldehyde for 10 minutes. Slides were then immersed in antigen retrieval buffer (10 mM sodium citrate, 0.05% Tween-20, pH 6) at 95-100° C., for 40 minutes, except for sections undergoing Pax7/Ertr7 co-labeling this step was omitted. Sections were then treated with blocking buffer from a mouse-on-mouse immunohistochemistry kit (M.O.M, kit; Vector) for 1 hour and immunolabeled with mouse anti-Pax7 and rabbit anti-HSP47 (1:200, Abcam, #77609) or anti-Ertr7 (1:1000, SCBT, #SC-73355) overnight at 4° C. Anti-Pax7 was purified from hybridoma cell supernatant (Developmental Studies Hybridoma Bank, Iowa City, Iowa) 68. Sections were washed with PBS and then incubated with horse anti-mouse IgG Dylight 594 (1:200, 30 min at RT. Vector, DI-2594) and horse anti-rabbit IgG Dylight 488 (1:200, 30 min at RT).

For identification of FAPs, sections were fixed in ice-cold acetone for 10 minutes and then incubated with blocking buffer for 1 hour. Sections were then labeled with rat anti-mouse CD31 conjugated with FITC (1:50, eBioscience, clone 390), rat anti-mouse CD45 conjugated with FITC (1:100, eBioscience, clone 30-F11), and goat anti-PDGFRα (1:100. R&D Systems, #AF1062) at 4° C., overnight. Sections were washed with PBS and then incubated with horse anti-goat IgG Dylight 594 (1:200, 30 min at RT, Vector).

For identification of fibrogenic PDGFRα+ cells, sections were fixed in ice-cold acetone for 10 minutes and then incubated with blocking buffer for 1 hour. Sections were then labeled with rabbit anti-mouse HSP47 (1:100, ABeam, clone EPR4217) and goat anti-PDGFRα at 4′C overnight. Sections were washed with PBS and then incubated with horse anti-goat IgG Dylight 594 (1:200, 30 min at RT) and horse anti-rabbit IgG Dylight 488 (1:200, 30 min at RT).

Stereology

The number of cells per volume of muscle was determined by measuring the total volume of each section using a stereological, point-counting technique to determine section area and then multiplying that value by the section thickness (10 μm). The numbers of immunolabeled cells in each section were counted and expressed as the number of cells per unit volume of each section.

Assays for Fiber Number, Central Nucleation and Size

TA muscles were sectioned at the midbelly of muscles and used for fiber cross-sectional area measurements-. The proportion of fibers containing central nuclei, an indicator of fiber regeneration, was also determined. Central-nucleation was expressed as the ratio of central nucleated fibers relative to the entire population of fibers sampled for each muscle. The cross-sectional areas of >300 muscle fibers were measured using a digital imaging system (Bioquant).

Assay of Muscle Connective Tissue Content

The volume fraction of muscle that was occupied by collagen types 1, 3 and 5 was determined by overlaying a 10×10 eye-piece grid on microscopic images of cross-sections of entire muscle that were immunolabeled with antibodies to collagen types 1, 3 or 5.

Preparation of BMMCs and BMDMs

BMMCs were separated from whole BMC preparations flushed from WT or CD11b/LIF femurs and tibiae and separated using a histopaque-1077 gradient (Sigma). The freshly-isolated BMMCs were then used for RNA isolation and analysis. For preparation of BMDMs, BMCs were aseptically flushed from WT or CD11b/LIF femurs and tibiae and differentiated in vitro to BMDMs24. BMDMs were stimulated for 24-hours with activation media consisting of Dulbecco's Modified Eagle Medium (DMEM) with 0.25% heat-inactivated fetal bovine serum (FBS; Omega), 100 U/ml penicillin, 100 μg/ml streptomycin (1% P/S), and 10 ng/ml macrophage colony stimulating factor (MCSF; R&D).

ELISA Analysis of BMDM Conditioned Media

Cultures of BMDM from WT mice were established as described above. On the sixth day of culture, the BMDMs were switched to DMEM containing 1% P/S, 0.25% heat-inactivated FBS and M-CSF or media containing 10 ng/ml recombinant mouse LIF (eBioscience) (stimulation media). After 24 hours of stimulation, conditioned media (CM) were collected, briefly centrifuged to remove particulates and then frozen at −20° C. Separate aliquots of BMDM CM from each sample were analyzed for expression of CCL2 (Duoset ELISA, R&D Systems, #DY479) and TGFβ (Emax immunoassay; Promega, #G7590), according to manufacturer's instructions. Following an HRP-based reaction, a colored product was formed in proportion to the amount of cytokine present, which was analyzed by a spectrophotometer (Bio-Rad Benchmark Microplate Reader) at a wavelength of 450 nm with a 540 nm correction. The concentration of each cytokine was determined by comparing the optical density of the samples to the standard curve.

ELISA Analysis of Serum

Whole blood was collected from the femoral artery and allowed to clot on ice for at least 30 min. The whole blood was spun for 10 min at 2000×g at 4° C. The serum was collected and stored in liquid nitrogen until analyzed for circulating LIF, TNFα, IFNγ, IL-4 and IL-10 by ELISA, according to manufacturer's instructions (R&D Systems, Quantikine ELISAs, #MLF00, MTA00B, MIF00, M4000B and M1000B). Each group contained 3 replicates.

Assays for LIF Effects on Muscle Cell Fibrogenesis

The C2C12 cell line was purchased from American Type Culture Collection (ATCC CRL-1772 cell line). The cells were authenticated as myoblasts by confirming their differentiation into contractile myotubes that express characteristic muscle proteins. Cells were seeded in six-well plates at 6×104 cells per well and cultured in DMEM containing 10% FBS, 1% P/S at 37′C in 5% CO2 for 24 hours and then serum-starved overnight prior to stimulation. To generate myotubes, myoblasts were grown to 90% confluence and then differentiated in serum-free DMEM for 24 hours. The cells were then returned to DMEM containing 10% FBS for 5 days. Myoblast and myotube cultures were stimulated with vehicle, TGFβ1 (10 ng/ml). LIF (10 ng/ml) or TGFβ1+LIF for 3- or 24-hours.

### FAPs and Myogenic Progenitor Cell Preparation and Isolation

FAPs were isolated from 6-month-old WT mice. Hindlimb and forelimb muscles were dissected and rinsed in DMEM. Muscles were minced and digested in 5 ml of enzyme buffer (DMEM, 25 mM HEPES (Sigma), 5 mM MgCl2 (Fisher), 2% P/S, 12.5 U dispase, type II, 12.5 U collagenase B and 20 μg/ml DNase I (Roche)) for 60 min at 37° C., with gentle trituration. The digestion was neutralized with 2 volumes of staining buffer (DMEM, 10 mM NaHCO₃(EMD Millipore), 25 mM HEPES, 5 mM EDTA, 5 mM MgCl2, 1 mM L-glutamine, 2% BSA and 2% P/S). The digestate was passed through 100 μm mesh filters and cells were pelleted at 350 g for 5 min. Cells were resuspended in ACK lysis buffer (Lonza) for 5 min followed by the addition of an equal volume of staining buffer and cells were pelleted at 350 g for 5 min. Cells were resuspended in staining buffer with CD16/32 (eBioscience #14-0161-85; 0.5 μg/test) for 10 min to block Fc receptor binding. Cells were labeled with Hoechst (Sigma #14533) and antibodies against CD11b (eBioscience #11-0112; 0.25 μg/test). CD31 (eBioscience #11-0311; 0.5 μg/test) and CD45 (eBioscience #11-0451; 0.1 μg/test) conjugated with FITC and PDGFRα conjugated with PE-Cy7 (eBioscience #25-1401; 0.2 μg/test). FAPs (Hoechst+CD11b/31/45-PDGFRα+) were sorted into collection buffer (DMEM, 10 mM NaHCO₃ and 20% FBS) using a BD SORP FACSAriaII cell sorter.

MPCs were isolated from 14-months-old CD11b/LIF mdx mice and littermate controls. Hindlimb and forelimb muscles were dissected and digested as described for FAPs isolation. Isolated cells were resuspended in staining buffer with CD16/32 for 10 min to block Fc receptor binding. Cells were labeled with cell impermeant dye DAPI (Sigma) to distinguish live cells and antibodies against CD11, CD31 and CD45 conjugated with FITC and Sca-1 conjugated with PE-Cy5 (eBioscience #15-5981; 0.2 μg/test), integrin a7 conjugated with PE (Medical and Biological Laboratories #K0046-5; 15 μl/test). Live myogenic progenitor cells (DAPI-CD11b/31/45-Sca1-α7int+) were sorted into Buffer RLT RNA lysis buffer (Qiagen) using a FACSAriaIII high speed cell sorter.

Primary Fibroblast Cell Culture

Sorted FAPs were cultured in growth medium (DMEM, 20% FBS, 10% heat-inactivated horse serum, 1% P/S and 2.5 ng/ml bFGF) on tissue culture plates coated with Matrigel41. After plating, cells were cultured for 3-days and half the medium was changed. Cells were expanded and subcultured. Prior to stimulation, cells were cultured in reduced serum media overnight (DMEM, 2% FBS, 1% P/S and 2.5 ng/ml bFGF). Fibrogenic cell cultures were stimulated with vehicle, TGFβ1 (10 ng/ml), LIF (10 ng/ml) or TGFβ1+LIF for 3-hours or 3-days (with media changes at 24- and 48-hours).

Physiological Analysis

Muscle stiffness and viscoelasticity at 14-months of age was assayed because connective tissue accumulation in mdv muscle is progressive between 3- and 24-months of age. It was expected that when sampling for effects of the transgene on muscle stiffness during the late, progressive stage of the disease, the magnitude of the effect would be greater, which would more definitively the question of whether the transgene influenced muscle stiffness. Male WT/mdx and LTF/mdx mice were anesthetized by the intraperitoneal (i.p.) injection of ketamine (40 mg/kg body weight). Anesthesia was checked by testing mice for a positive reflex response to a hind foot pinch and by monitoring respiration. Additional i.p, injections of ketamine were given throughout the study, as needed. For in-situ analysis of the TA muscle the knee was immobilized to the heated (37° C.) platform of an 809C in-situ mouse apparatus (Aurora Scientific). Silk sutures (5-0; Ethicon) were knotted to the distal, severed tendon and then secured to the lever arm of a dual-mode force transducer-servomotor (Aurora Scientific, Model 305C-5N). After placing platinum-tipped electrodes into the leg above the knee, flanking the sciatic nerve, the TA muscle was stimulated by pulses and manipulated on three axes to find the optimal muscle length (Lo). Lo was multiplied by the pennation of 0.6 for the TA muscle⁶⁹ to determine optimal fiber length (Lf). To measure elasticity, the muscle was left unstimulated while the lever arm oscillated at ±20% of the Lf for 20 seconds. Muscles were allowed to rest for 60 seconds before subsequent oscillation series. Muscles were allowed to rest for 60 seconds before a series of oscillations at 3 Hz, which provides a physiological strain and strain rate⁷⁰. Dynamic Muscle Control and Dynamic Muscle Analysis (Aurora Scientific) software was used to conduct experiments and record data. Force measurements were normalized to muscle cross-sectional area, and position measurements were normalized to percent of Lf.

Statistical Analyses

All data are presented as mean±sem. Statistical significance was calculated using unpaired Student's t-tests or ordinary one-way ANOVA with Tukey's multiple comparison test to determine differences among multiple groups. Differences with a P-value <0.05 were considered statistically significant. The equality of variance between the groups that are being compared was tested with an F test, experiments with a P-value <0.05 are denoted in the figure legend. Additionally, for immunohistochemistry and immunofluorescence experiments, slides were only included if concurrently immunolabeled. Statistical analysis was performed using GraphPad Prism.

### Results

A CD11b Regulated LIF Transgene Suppresses M2-Biased Markers

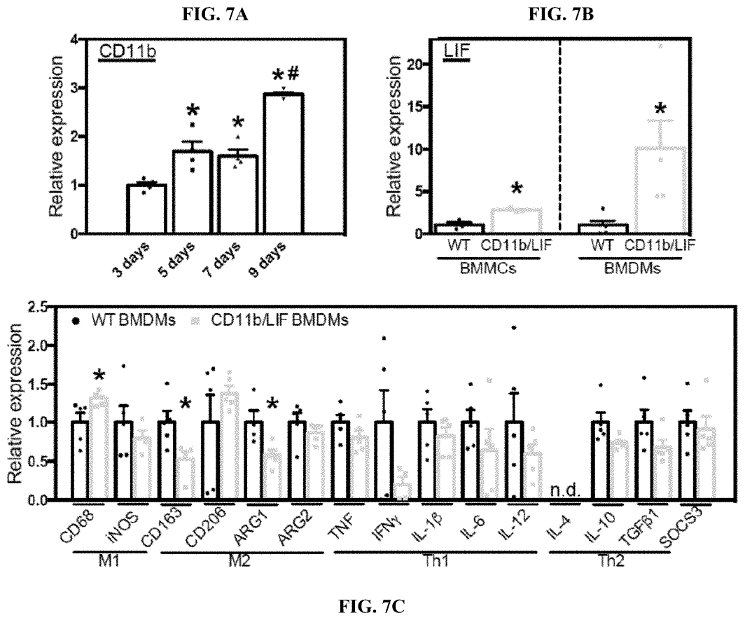

Mice were generated with a LIF transgene under control of the CD11b promoter (CD11b/LIF transgenic mice). Quantitative PCR (QPCR) analysis of Cd11b mRNA levels confirmed that Cd11b expression increased as BMCs differentiate into bone marrow-derived macrophages (BMDMs) (FIG. 7A). Freshly-isolated bone marrow mononuclear cells (BMMCs) from transgenic mice had a ˜2.8-fold higher Lif expression compared to wild-type (WT). After 9 days of culture, Lif expression was ˜10-fold higher in transgenic BMDMs than WT (FIG. 7B). Thus, LIF transgene expression increased with increased CD11 promoter activation as monocytes differentiate into mature macrophages. Upon becoming fully-differentiated macrophages, the CD11b/LIF transgene had an autocrine effect on macrophage phenotype, increasing expression of Cd68 by ˜31% and reducing Cd163 and arginase-1 (Arg1) by 47% and 42%, respectively (FIG. 7C). CD68 is present at high levels in macrophages that are biased to a pro-inflammatory phenotype (M1-biased). Arginase and CD163 are present in macrophages that are biased toward a pro-fibrotic and reparative phenotype (M2-biased)²².

CD11b/LIF Transgene Reduces Mdx Muscle Inflammation and Fibrosis

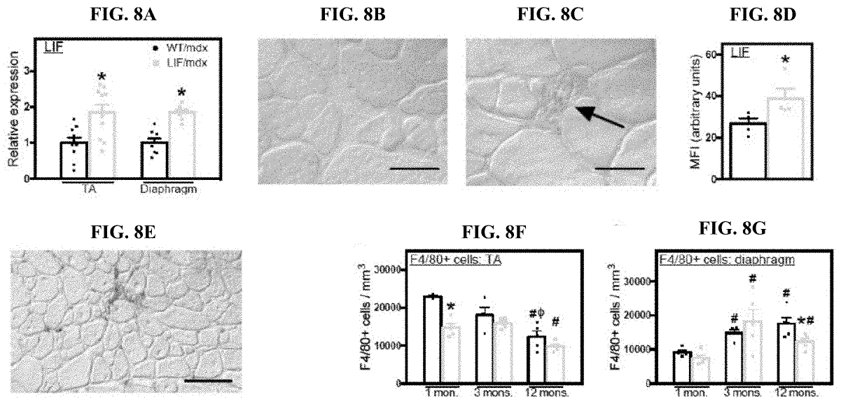

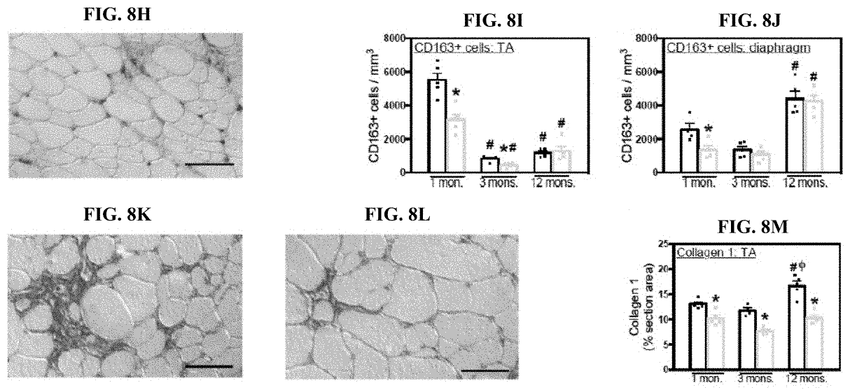

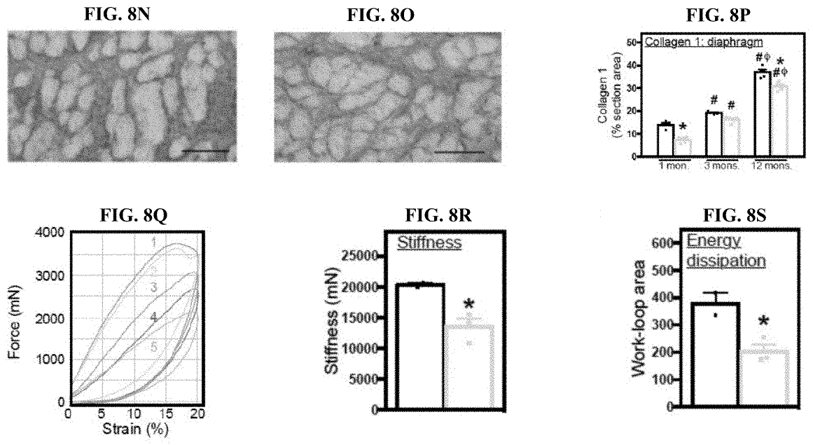

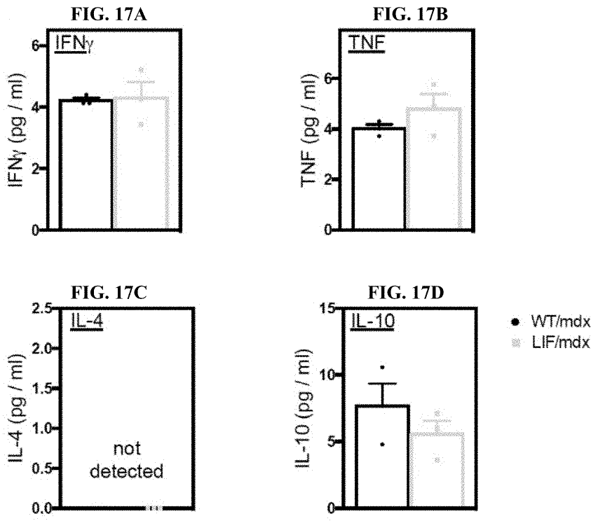

It was assayed whether expression of the CD11b/LIF transgene affected mdx pathology, focusing on influences on muscle inflammation and fibrosis. It was confirmed that elevated expression of Lif in the tibialis anterior (TA) and diaphragm muscles of transgenic mice (CD11b/LIF mdx mice) (FIG. 8A) and observed that cells in inflammatory lesions in CD11b/LIF mdx mice showed higher levels of LIF protein than non-transgenic mice (FIGS. 8B-8D). However, sera from transgenic mice showed no elevation in LIF protein assayed by ELISA (mean±sem: WT/mdx 19.25±1.85 and LIF/mdx 26.19±4.86 cells/mm³, n=3 per data set. P=0.25: two-tailed t-test). There were no significant differences in the concentrations of cytokines previously implicated in influencing the pathology of muscular dystrophy (IFNγ, TNF, IL-4 and IL-10) in the serum of transgenic mice, compared to non-transgenic mice (FIG. 17).

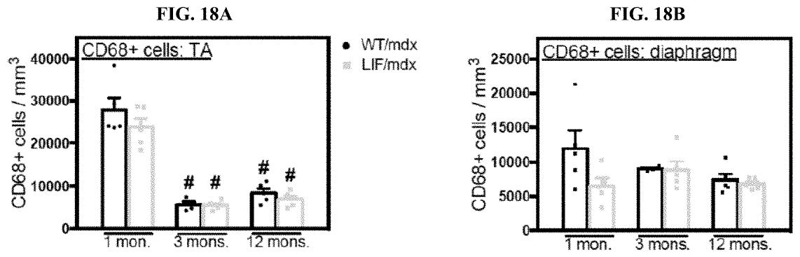

Effects of the transgene on mdx pathology were assessed over the course of the disease, sampling at the acute onset of pathology (1-month-old), the period of successful regeneration (3-months-old) and the late, progressive stage of pathology (12-months-old) in TA muscles. Diaphragm muscles show a progressive pathology following disease onset. The CD11b/LIF transgene reduced numbers of macrophages expressing the pan-macrophage marker F4/80 at the stages of pathology characterized by extensive, muscle inflammation (1-month-old in TA; 12-months-old in diaphragm) (FIGS. 8F-8G). The transgene also reduced numbers of CD163+ macrophages at the acute onset of pathology in both TA and diaphragm (FIGS. 8H-8J) but did not affect numbers of CD68+ macrophages in either muscle at any stage of the disease that was tested (FIG. 18).

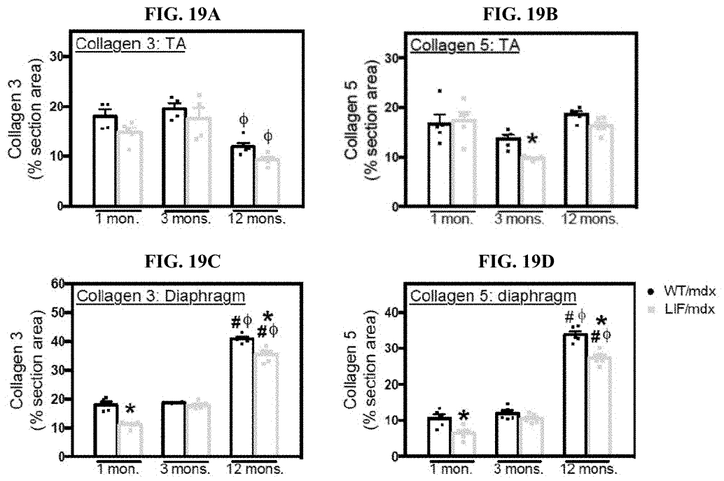

It was tested whether the CD11b/LIF transgene reduced collagen accumulation in mdx muscles, which would be consistent with a reduction in numbers or activity of CD163 macrophages that promote fibrosis of dystrophic muscle²³. Both the TA and diaphragm showed significant reductions in collagen type 1 at the acute onset of pathology, and the transgene completely abrogated collagen type 1 accumulation in the TA muscle, at least until 12-months-old (FIGS. 8K-8M). The CD11b/LIF transgene also reduced accumulation of collagen type 1 in diaphragms (FIGS. 8N-8P) and reduced accumulation of collagen types 3 and 5 in diaphragms at late stages of pathology and reduced collagen type 5 in 3-month-old TA muscles (FIG. 19).

Because the CD11b/LIF transgene prevented collagen type 1 accumulation in TA muscles and collagen type 1 is primarily responsible for increased muscle stiffness caused by fibrosis, changes in the passive mechanical properties of TA muscles in CD11b/LIF transgenic mdx mice were assayed. TA muscles were subjected to cyclic, dynamic loading using 20% strains at a 0.6/second strain rate, which is within the physiological range. Lissajous figures obtained by measuring force-strain relationships showed that muscle stiffness (indicated by the slope of the tangent to the loading phase of each cycle) was significantly less in CD11b/LIF transgenic mdx mice (FIG. 8Q, FIG. 8R). In addition, the transgenic mdx muscles showed less energy dissipation during each cycle of loading (proportional to the area inside each hysteresis loop during a cycle of loading/unloading) (FIG. 8Q, FIG. 8S), indicating higher mechanical efficiency in the CD11b/LIF transgenic muscles.

CD11b/LIF Transgene does not Impair Muscle Growth

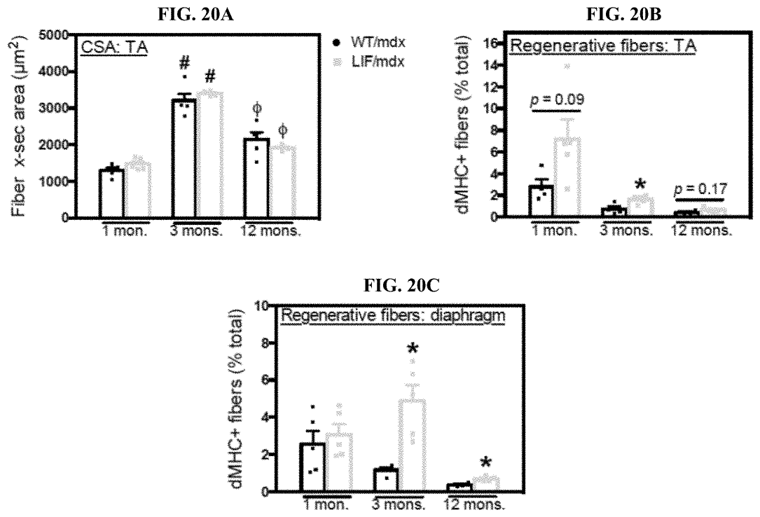

Although previous investigations showed that M2-biased macrophages promote muscle fibrosis, they also promote regeneration^{23,24}. This possibility was tested by assaying for effects of the transgene on TA muscle fiber cross-sectional area as an index of regeneration and found no difference in TA fiber size between transgenic and non-transgenic mdx mice at any age sampled (FIG. 20A). The proportion of muscle fibers that expressed developmental myosin heavy chain (dMHC), which is upregulated in regenerating fibers, was also assayed. A higher proportion of dMHC+ fibers in TAs of CD11b/LIF transgenic mdx mice at 3-months-old and a trend for more dMHC+ fibers at 1-month and 12-months-old, compared to non-transgenic mdx mice were observed (FIG. 20B). Similarly, the proportions of dMHC+ fibers in 3-months-old and 12-months-old diaphragms were increased by the transgene (FIG. 20C). Collectively, these observations indicate that the CD11/LIF transgene does not impair muscle growth or regeneration, despite the reduction of CD163+ cells.

Transplanted CD11b/LIF Cells Reduce Inflammation

The analyses of CD11b/LIF transgenic mdx mice showed that the transgene reduces muscle inflammation and fibrosis, thereby validating the transgene as a therapeutic molecule for muscular dystrophy. However, the primary goal in the investigation was to determine whether transplanted bone marrow derived cells (BMDCs) could serve as vehicles to deliver therapeutic molecules to dystrophic muscle through a clinically-relevant approach; in particular, it was questioned whether transplantation of genetically-modified BMCs into dystrophic animals provides a strategy for targeted delivery of therapeutic cargo to diseased muscle. Treatment effects in 6-months-old mdx mice at 4-months post-bone marrow transplantation (BMT) was assayed for scientific and technical reasons. First, it was anticipated that a likely, beneficial outcome of leukocyte delivery of a LIF transgene to muscle would be reductions in fibrosis. Previous work⁵ showed that 6-months-old mdx muscles show significantly elevated accumulation of type I and type III collagen. It was also shown that at 6-months-old, mdx limb muscles contain elevated numbers of M2-biased macrophages that contribute to muscle fibrosis⁵. The technical rationale for sampling at 6-months is that engraftment of transplanted cells takes time and preliminary experiments showed that high levels of engraftment could be achieved by 4-months post-BMT.

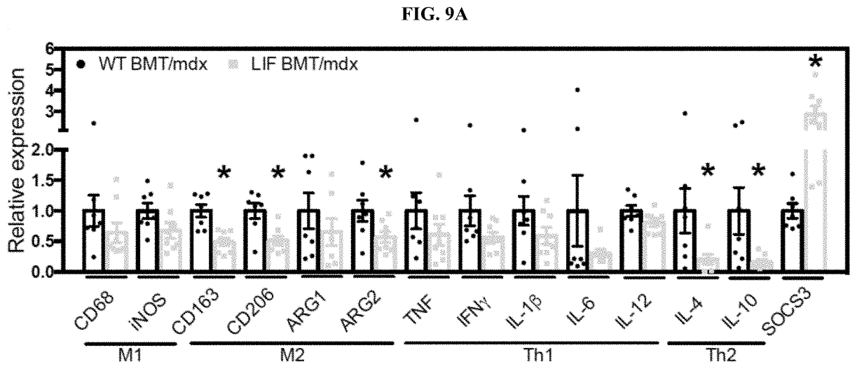

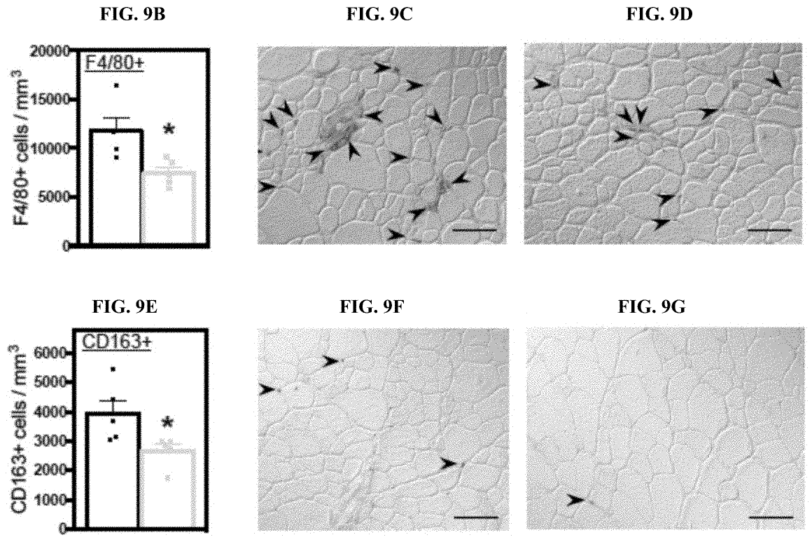

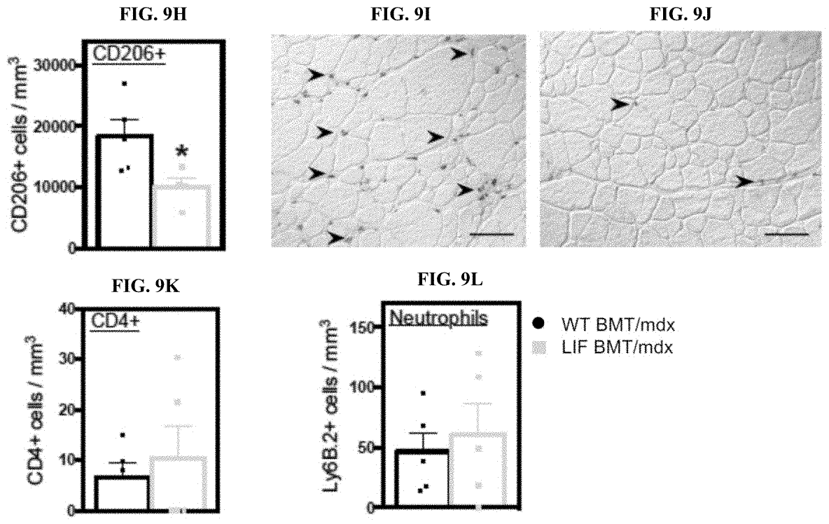

At the time of tissue collection from transplant recipients, circulating leukocytes were 86.6% donor-derived (sem=1.14: n=25). QPCR of muscles showed that CD11b/LIF recipients (LIF BMT/mdx mice) had reduced expression of the M2-biased markers Cd163, CD206 (Mrc1) and arginase-2 (Arg2) expression by 51%, 49% and 43%, respectively (FIG. 9A). This effect resembles the autocrine effect of CD11b/LIF on macrophages in vitro (FIG. 7C). Additionally, the transgene affected the expression of Th2 cytokines associated with M2-biased macrophage activation, IL-4 (Il4) and IL-10 (Il10), which were reduced by ˜79% and ˜84%, respectively (FIG. 9A). Reduced cytokine expression was accompanied by a ˜2.8-fold increase in the expression of suppressor of cytokine signaling 3 (Socs3) in CD11b/LIF BMT recipients (FIG. 9A). Socs3 expression is activated by LIF²⁵ and its elevation in muscles of CD11/LIF BMT recipients verifies an increase in LIF signaling in muscle.

The effect of CD11b/LIF BMT on macrophage numbers and phenotype was tested because changes in macrophages have profound effects on dystrophic muscle pathology^{7,13,14,26}. Immunohistochemistry using anti-F4/80, to identify total macrophage populations, or anti-CD68 (M1-biased macrophages), anti-CD163 (M2-biased), or anti-CD206 (M2-biased) was performed. Mdx mice that received CD11b/LIF BMCs showed a 37% fewer F4/80+ cells compared to mice receiving WT BMCs (FIG. 9B). Quantitation of CD68+, CD163+ and CD206+ macrophages showed no difference in CD68+ cells (mean±sem: WT BMT/mdx 17525±1502 and LIF BMT/mdx 16377±1440 cells/mm³, n=5 per data set, P=0.60; two-tailed t-test), a 32% reduction of CD163+ cells (FIG. 9E) and 46% fewer CD206+ cells (FIG. 9H) in the dystrophic muscle. However, numbers of CD4+ T-cells and neutrophils in mdx muscles were unaffected by transplantation of CD11b/LiF BMCs (FIG. 9K, FIG. 9L), indicating a selective reduction of M2-biased macrophages caused by transgenic BMDCs.

LIF Reduces Ccl2 Expression in Muscle and Macrophages

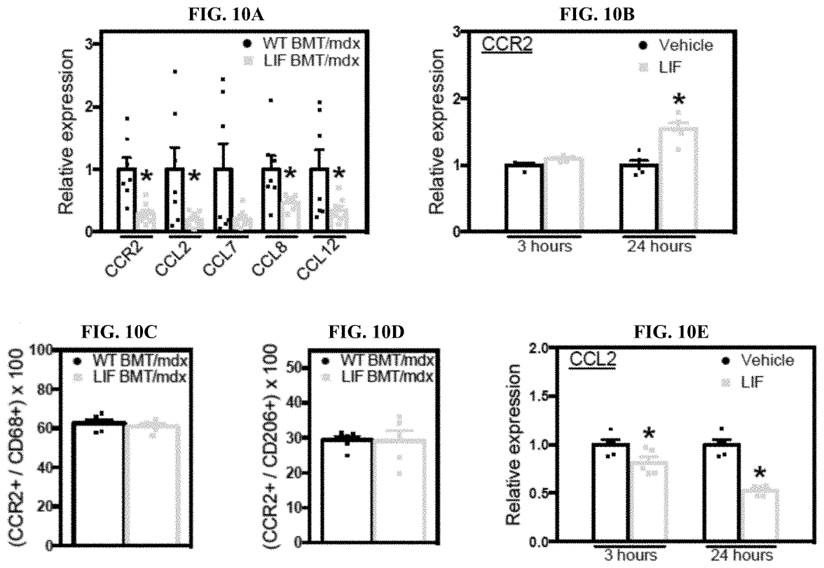

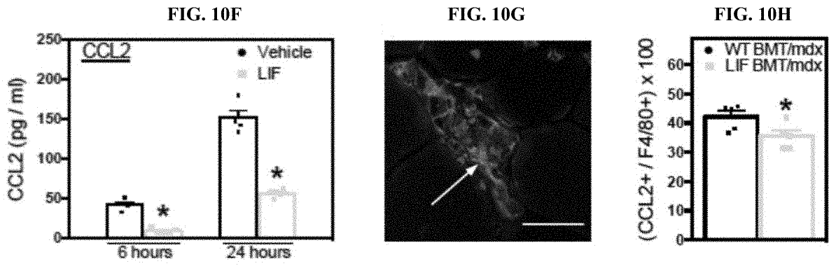

The large reductions of M2-biased macrophages in dystrophic muscle of mice transplanted with CD11b/LiF BMCs (FIG. 9) suggest that LIF inhibits their recruitment. Because abrogation of CCR2 signaling reduces macrophage accumulation in dystrophic muscle²⁷, whether CCR2 signaling was affected by LIF was tested. QPCR assays showed reduced expression of Ccr2 and its ligands Ccl2, Ccl8 and (c112 in muscles of CD11b/LIF BMT recipients, and a strong trend for lower levels of Ccl7 expression (P=0.06) (FIG. 10A).

Next, the possibility that LIF acts directly on macrophages to inhibit CCR2 signaling in vitro was tested. Unexpectedly, brief periods of macrophage stimulation with LIF had no effect on Ccr2 expression and extended periods significantly increased Ccr2 expression (FIG. 10B). It was assayed whether the CD11b/LIF transgene affected the numbers of intramuscular macrophages that expressed detectible CCR2 but found that the proportion of CD68+ or CD206+ macrophages that expressed CCR2 was not influenced by the transgene (FIG. 10C, FIG. 10D). This indicates that reductions in macrophage-derived CCR2 in muscles reflect reductions in macrophage numbers, rather than ablating the expression of CCR2 in macrophages in CD11b/LIF BMT recipients. However, stimulation of BMDMs with LIF reduced Ccl2 expression and CCL2 protein secretion (FIG. 10E, FIG. 10F), indicating that LIF acts directly on macrophages to negatively regulate Ccl2. In addition, F4/80+ macrophages were prominent sources of CCL2 in nd % muscle (FIG. 10G), and transplantation of CD11b/LIF BMCs reduced the proportion of F4/80+ macrophages that expressed detectible CCL2 by 15% (FIG. 10H).

Transplanted CD11b/LIF Cells Reduce Muscle Fibrosis

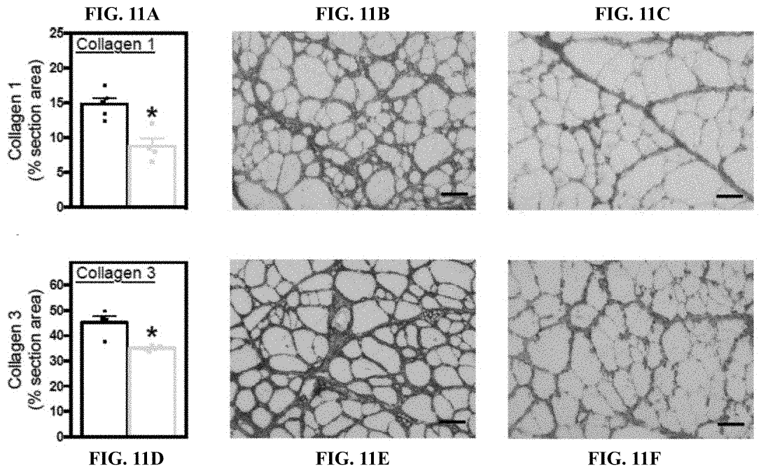

Fibrosis of dystrophin-deficient muscle is largely driven by arginine metabolism by arginase expressed by M2-biased macrophages²³. Arginine hydrolysis by arginase produces metabolites that are utilized to generate substrate molecules necessary for connective tissue production's. Because reductions in M2-biased macrophages in muscles of mdv mice that were recipients of CD11b/LIF BMT and lower levels of expression of Arg2 were observed, it was assayed whether fibrosis was affected. Transplantation of CD11b/LIF BMCs reduced collagen types 1, 3 and 5 in mdx muscle by ˜41%, 22% and 25%, respectively, compared to WT BMT recipients (FIGS. 11A-11I). However, the anti-fibrotic effect of CD11b/LIF BMT cannot be solely attributed to reductions of arginine metabolism by M2-biased macrophages. QPCR data showed that mRNA levels of collagen types 1 alpha 1 (Col1a1), 3 alpha 1 (Col3a1) and 5 alpha 3 (Col5a3) were reduced by ˜57%, 51% and 30%, respectively, in CD11b/LIF BMC recipients (FIG. 11J), indicating treatment effects on fibrogenic cells, in addition to effects on macrophages that provide substrate for fibrogenesis.

M2-biased macrophages can act directly on fibrogenic cells through TGFβ which activates fibro/adipogenic progenitor cells (FAN) into fibroblasts and stimulates fibroblasts to produce collagen²⁹⁻³². TGFβ can also activate Wnt-signaling, which increases myogenic-to-fibrogenic conversion of muscle stem cells, further contributing to dystrophic muscle fibrosis³³. It was tested whether the CD11b/LIF BMT affected key transcripts of the Wnt and TGFβ pro-fibrotic pathways. Although there was no effect on expression of Tgfb1 or Axin2, a Wnt-target gene (FIG. 11K), the expression of downstream TGFβ target genes connective tissue growth factor (Ctgf), fibronectin (Fn1) and snai1 family zinc finger 1 (Snai1)³⁴⁻³⁷ were reduced by ˜33%, 43% and 33%, respectively. (FIG. 11K).

LIF Reduces Macrophage TGFβ1 Expression

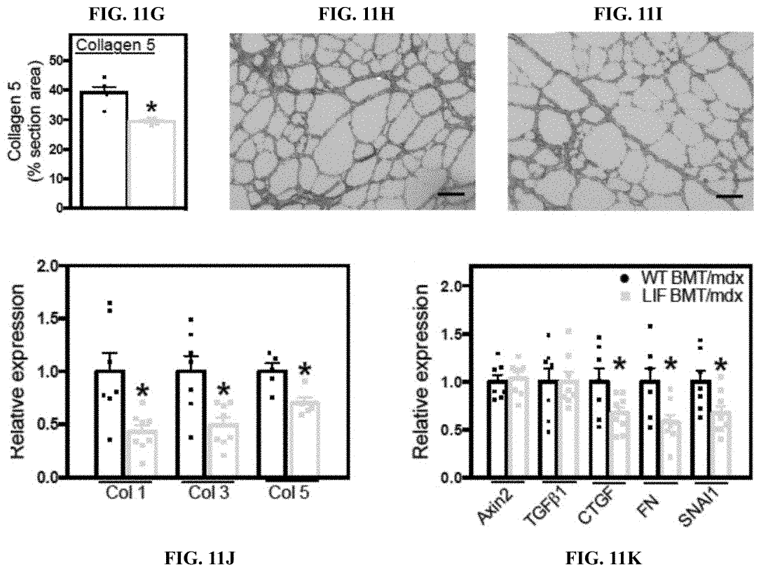

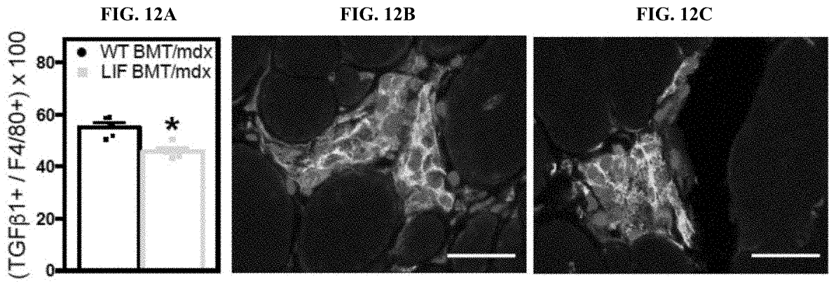

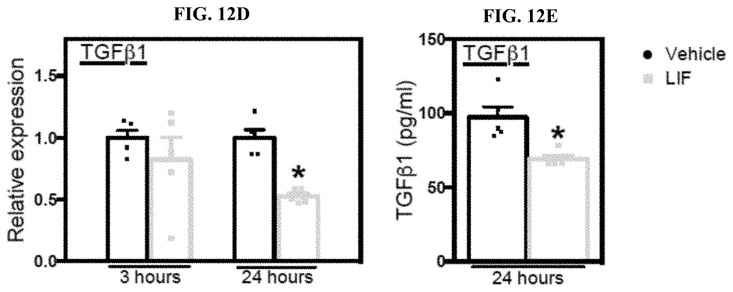

Although no effect of CD11b/LIF BMT on Tgfb1 mRNA in whole muscle homogenates was observed, effects on TGFβ expression in intramuscular macrophages were assayed more specifically by assaying the proportion of macrophages that expressed TGFβ. It was found that there were 17.7% fewer intramuscular macrophages that expressed detectible TGFβ in CD11b/LIF recipients, compared to WT recipients (FIG. 12A). Interestingly, the greatest reduction of TGFβ expressing macrophages was seen in inflammatory lesions of CD11b/LIF recipients (FIG. 12C) compared to WT recipients (FIG. 12B).

It was tested whether reduced TGFβ1 expression in CD11/LIF BMT recipients reflected direct actions of LIF on macrophages to inhibit TGFβ1 expression. When BMDMs were treated with LIF for 24 hours, Tgfb1 gene expression was reduced by 47% and secreted TGFβ protein expression by 29% (FIG. 12D, FIG. 12E), showing that LIF is a negative regulator of TGFβ1 expression in macrophages. However, Tgfb1 gene expression was not reduced after 3 hours of LIF stimulation, suggesting that LIF-mediated inhibition of Tgfb1 could be a secondary effect.

LIF Reduces Fibrogenesis and Ctgf mRNA in Muscle Cells

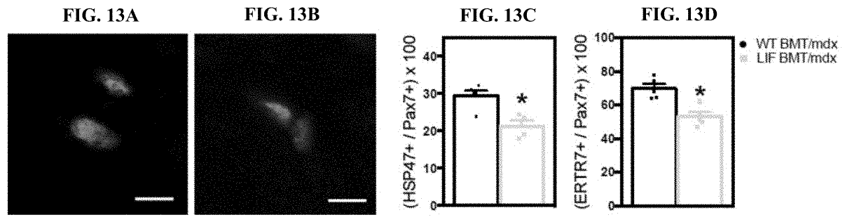

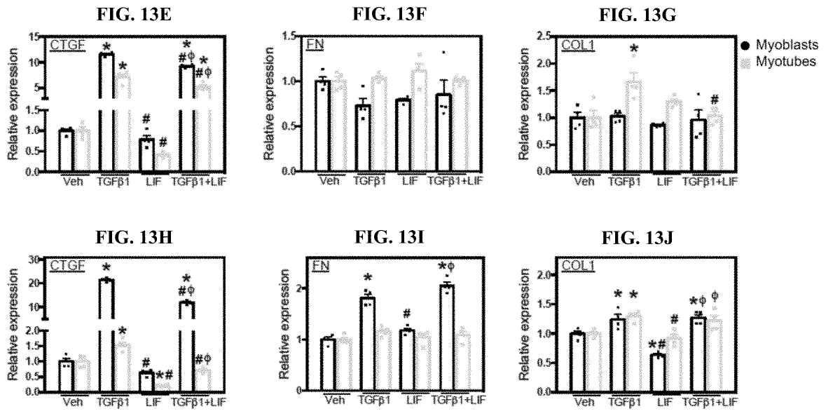

TGFβ signaling promotes the fibrogenic conversion of myogenic cells in dystrophic muscle, thereby contributing to fibrosis³³. Because transplantation of CD11b/LIF BMCs into mdx mice reduces fibrosis, it was tested whether LIF reduces the proportion of myogenic cells acquiring a fibrogenic phenotype. Muscle sections that were double-labeled with anti-Pax7, a marker of satellite cells, and anti-HSP47, a collagen-specific molecular chaperone^{38,39} showed that the proportion of Pax7+ cells that expressed HSP47 was reduced by 27.8% in CD11b/LIF recipients (FIGS. 13A-13C); this indicates that satellite cells had a less fibrogenic phenotype in CD11b/LIF recipients. Expression of Serpinh1, the gene that encodes HSP47, was also reduced 24% in the whole muscle lysate of CD11b/LIF recipients (mean±sem: WT BMT/mdx 1±0.08 and LIF BMT/mdx 0.76±0.06, n=7 and 8 per data set, respectively, P=0.03; two-tailed t-test). It was also assayed whether transplantation of CD11b/LIF BMCs affected the proportion of satellite cells that expressed ERTR7 in vivo. ERTR7 was chosen in addition to HSP47 because satellite cells in injured and aging muscle that display elevated levels of ERTR7 expression have shifted away from a myogenic phenotype, toward a fibrogenic phenotype^{40,41}. The data show that the transgene reduced the proportion of satellite cells that expressed ERTR7 in mdx muscle in vivo, similar to the reduction of satellite cells expressing HSP47 (FIG. 13C, FIG. 13D).

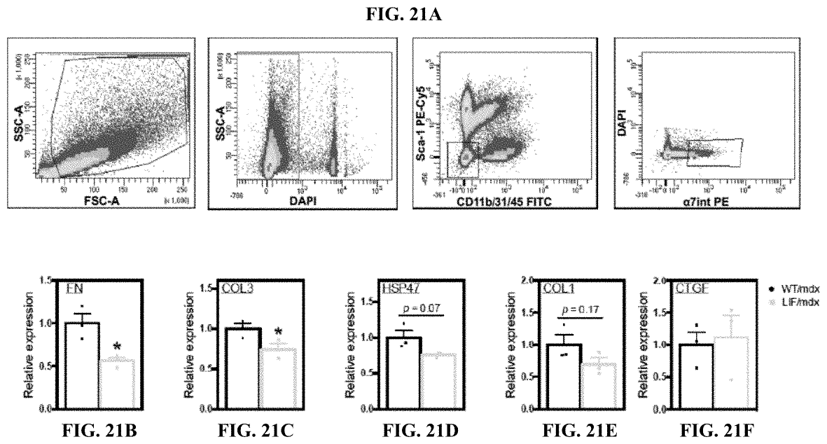

It was also tested whether the CD11b/LIF transgene affected the phenotype of myogenic progenitor cells (MPCs) in later stages of mdx pathology by assaying for changes in the expression of fibrogenic genes in MPCs that were freshly-isolated from muscles of 14-months-old mice. MPCs (CD11b-CD31-CD45-Sca1-7 integrin+ cells) from CD11b/LIF transgenic mdx mice showed lower expression levels of Fn1 and Col3a1 compared to non-transgenic mice (FIG. 21). In addition, strong trends for the reduction in expression of Serpinh1 (HSP47) and Col1a1 in freshly-isolated MPCs were observed.

The effects of LIF on TGFβ1-induced muscle cell fibrogenesis in vitro were examined. Myoblasts and myotubes treated with TGFβ1 and/or LIF were assayed for changes in expression of fibrogenic genes downregulated in CD11b/LIF BMT recipients (Ctgf. Fn1 and Col1a1; FIG. 11J, FIG. 11K). Co-stimulation with TGFβ1 and LIF inhibited Ctgf expression, compared to cells treated with TGFβ1 only (FIG. 13E, FIG. 13H). LIF also reduced basal Ctgf expression after 24 hours of stimulation in myotubes. Fn1 expression was stable in myoblasts treated with TGFβ1, LIF or TGFβ1+LIF for 3-hours (FIG. 13F). After 24-hours, TGFβ1-induced Fn1 expression, but co-stimulation with LIF had no effect (FIG. 13I). TGFβ1 stimulation for 3-hours induced the expression of Col1a1 in myotubes, and LIF attenuated TGFβ1-induced expression of Col1a1 in myotubes (FIG. 13G). LIF stimulation for 24-hours reduced basal Col1a1 expression in myoblasts, but not TGFβ1-induced expression of Col1a1 (FIG. 13J).

LIF Reduces the Prevalence of FAPs in Dystrophic Muscle

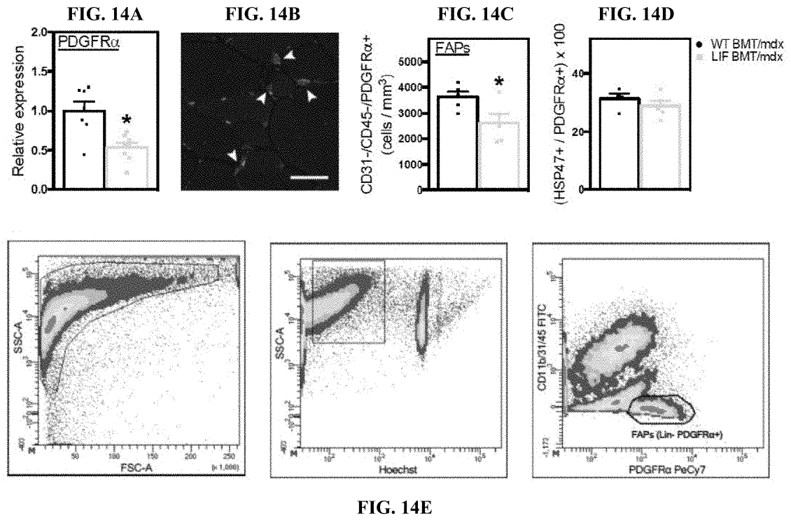

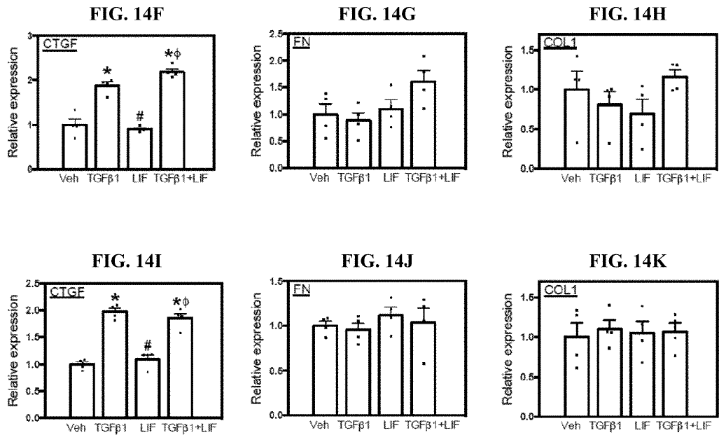

Because FAP-derived fibroblasts are important sources of connective tissue proteins, it was assayed whether CD11b/LIF BMT affected FAP numbers in vivo or whether LiF affected expression of fibrogenic proteins by FAP-derived fibroblasts in vitro. QPCR analysis showed that CD11b/LIF BMT recipients had a 47% reduction in Pdgfra expression (FIG. 14A) which could reflect fewer FAPs. Recipients of CD11b/LIF BMT had fewer cells that expressed PDGFRα and were double-negative for CD31 and CD45, which are FAPs⁴² (FIG. 14B, FIG. 14C), although the proportion of PDGFRα+ cells that expressed HSP47 was unaffected by the transgene (FIG. 14D). The findings indicate that reductions in numbers of FAPs in the muscles of mdx mice receiving CD11b/LIF BMT may contribute to reduced muscle fibrosis.

It was then tested whether LIF influenced the fibrogenic activity of FAP-derived fibroblasts in vitro. FAPs (CD11b/31/45−PDGFRα+) from WT muscles were sorted (FIG. 14E) and subcultured them prior to stimulation with TGFβ1, LIF or TGFβ1+LIF^{41,42}. Fibroblasts derived from FAPs were used rather than freshly-isolated FAPs because fibroblasts differentiated from FAPs are the primary source of connective tissue proteins in muscle³². It was tested if LIF affected Pdgfra expression in fibroblasts in vitro because enhanced PDGFRα signaling can cause pathological fibrosis⁴³. However, LIF did not affect Pdgfra expression in fibroblasts (mean±sem: control cells 1±0.04 and LIF-treated cells 1.13±0.23, n=4 per data set, P=0.61; two-tailed t-test). Treatments for 3-hours with TGFβ1 induced Ctgf expression in fibroblasts, but LIF had no effect on basal or TGFβ1-induced Ctgf (FIG. 14F). The magnitude of TGFβ1-induced Ctgf expression in fibroblasts (1.9-fold) was less than in myoblasts (˜11.6 fold) and myotubes (˜7.1-fold) (FIG. 13E). TGFβ1, LIF or TGFβ1+LIF had no effect on Fn1 or Col1a1 expression in fibroblasts (FIG. 14G, FIG. 14H). It was then tested whether prolonged stimulation of fibroblasts with TGFβ1, LIF or TGFβ1+LIF affected Ctgf, Fn1 or Col1a1 expression. Similar to effects of brief stimulations, Ctgf expression was induced ˜2.0-fold by TGFβ1 but the induction was not affected by LIF. There was also no effect of prolonged stimulation with TGFβ1 on the expression of Fn1 or Col1a1 (FIGS. 14I-14K).

Transplanted CD11b/LIF Cells do not Affect Muscle Growth

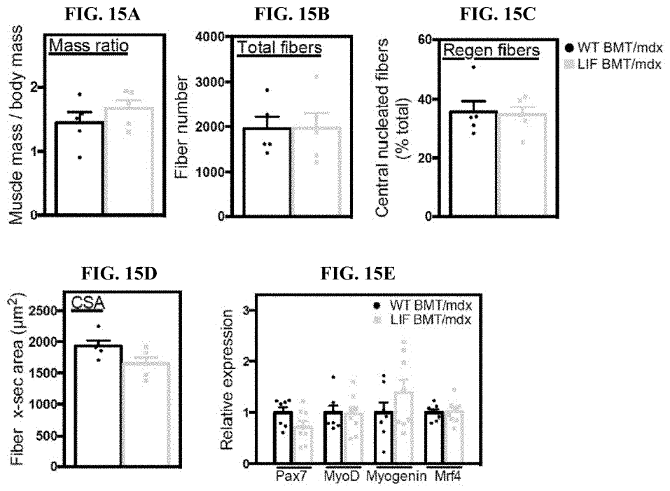

Because changes in macrophage phenotype and numbers influence muscle regeneration and myogenesis^{14,44-47}, it was assayed whether regeneration was affected in CD11b/LIF BMT recipients. There were no significant differences in TA muscle weight, total muscle fiber number, proportions of regenerating fibers or muscle fiber size (FIGS. 15A-15D). No muscle fibers expressed dMHC in WT BMT/mdx or LIF BMT/mdx mice. Additionally, QPCR assays showed no effect of CD11b/LIF on expression of the myogenic transcription factors: Pax7, Myod1, Myog or Mrf4 (FIG. 15E). These data indicate that the CD11b/LIF transgene did not influence processes through which immune cells modulate regeneration in mdx muscle.

### DISCUSSION

The results of the investigation demonstrate that transplantation of genetically-modified BMCs provides a means to deliver therapeutic molecules to dystrophic muscle. In addition, by regulating expression of the therapeutic transgene with the CD11b promoter, LIF delivery can be modified by the stages of maturation and activation of innate immune cells that differentiate from BMCs. This strategy provides a mechanism for the endogenous regulation of transgene expression by the transplant recipients that is responsive to the magnitude and site of inflammation. This system also permits long-term delivery of therapeutic molecules following a single therapeutic intervention. Although tissues were analyzed four months following transplantation in the present investigation, at that time circulating leukocyte populations were nearly 87% donor-derived. However, in humans experiencing BMT, stable mixed chimerism can persist for years in peripheral blood cell populations^{48,49}, showing that long-term benefits to humans can result from a single transplantation.

The potential therapeutic advantage of targeting therapeutic molecules to diseased tissue by using transgenes under control of the CD11b promoter is emphasized by comparing the findings with the outcomes of previous strategies to deliver LIF via hematopoietic cell transplantation. Transplantation of a hematopoietic cell line in which the cells were multiply-transduced with a retroviral construct containing cDNA encoding LIF produced high systemic levels of LIF and killed the recipient mice^{50,51}. In those experiments the retrovirus-transplant recipients reached serum LIF concentrations at 1400 units/ml, although serum LIF was undetectable in mice transplanted with cells that did not contain the LIF expressing retrovirus^{50,51}. This contrasts with the delivery system employed, in which elevated LIF production was detectible within inflammatory lesions in dystrophic muscle and pathology was reduced, but LIF remained undetectable in the sera. This indicates that more precise temporal and spatial delivery of LIF is necessary for safe and beneficial therapeutic application.

Exogenous LIF has been reported previously to increase growth of dystrophic muscle fibers^{19,20}, but an effect of the CD11b/LIF transgene on muscle mass or fiber size in transgenic mice or in CD11b/LIF BMT recipients was not observed. These differences in outcome may reflect the different modes of LIF delivery, in which increased fiber size resulted from continuous delivery of high concentrations of exogenous LIF^{19,20}. However, it was found that transplantation of CD11b/LIF transgenic BMCs affected mdx muscle by decreasing muscle fibrosis, consistent with the treatment effect achieved by delivery of exogenous LIF^{19,20,52}. In part, the anti-fibrotic influences of the CD11b/LIF transgene were attributable to modifying the phenotype of satellite cells, reflected in the reduced proportion of satellite cells that expressed detectible levels of the collagen chaperone, HSP47, and expressed ERTR7, a connective tissue protein expressed by pro-fibrotic satellite cells⁴⁰. This is functionally important in the context of DMD pathology because the transition of satellite cells from an HSP47-/ERTR7- to an HSP47+/ERTR7+ phenotype reflects a reduction in their myogenic capacity and an increase in their production of connective tissue proteins that may exacerbate the pathology of muscular dystrophy³³ and lead to a reduction in the regenerative capacity of muscle over time⁴⁰.

Although the CD11b/LIF transgene reduced the expression of pro-fibrotic molecules by muscle cells in CD11b/LIF BMT recipients in vivo, LIF did not reduce the basal level of expression of genes encoding connective tissue proteins by muscle cells in vitro. Instead, it was found that LIF reduced the activation of pro-fibrotic genes in myoblasts that was induced by the cytokine TGFβ. TGFβ has broad, profibrotic effects by increasing the expression of major, connective tissue proteins, including collagen and fibronectin^{53,54}, and reductions in TGFβ can significantly decrease fibrosis of dystrophin-deficient muscle, at least at early stages of the disease^{1,2,29}. In addition to increasing the production of connective tissue proteins, TGFβ can also influence muscle fibrosis by promoting the differentiation of myofibroblasts from muscle^{55,56} and by increasing the expression of other profibrotic growth factors, especially CTGF^{53,54}. The finding that LIF reduced or prevented the TGFβ-mediated induction of Ctgf expression in muscle cells may be particularly significant in mdx pathology because reductions in Ctgf expression can significantly slow pathology⁵⁷. Thus, the in vitro and in vivo data collectively indicate that increases in LIF diminish fibrosis of dystrophic muscle by opposing the profibrotic influence of TGFβ on muscle cells.

The observation that the CD11b/LIF BMT reduced TGFβ1 expression in intramuscular macrophages without causing reductions in total TGFβ1 expression in whole muscle also indicates the specificity of targeting treatment effects that are achieved by the CD11b/LIF transgene. This may provide advantages over other experimental and therapeutic approaches that have been explored previously to reduce fibrosis of dystrophic muscle by inhibiting TGFβ1 expression or activity through pharmacological approaches^{1,3,58,59}. While those pharmacological approaches are technically straight-forward and effective at reducing fibrosis in dystrophic muscle, their systemic administration does not provide delivery specifically to sites of inflammation, and increases the risks of off-target effects.

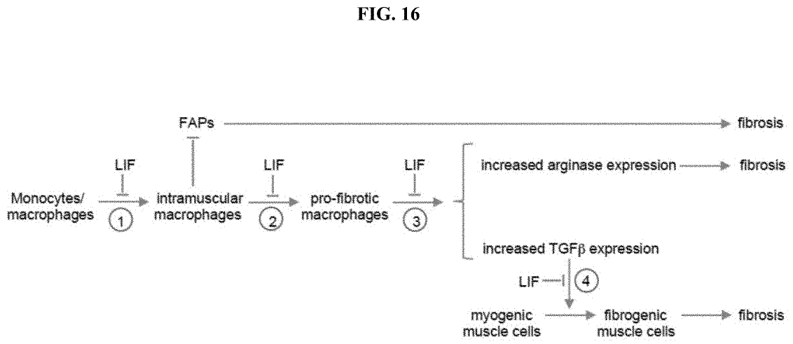

Although CD11b/LIF BMT reduced pathological changes in satellite cells, it was found that some beneficial effects of CD11b/LIF transgenic cells are attributable to modulation of the inflammatory response, rather than direct actions on muscle (FIG. 16). Despite the fact that DMD and mdx dystrophy result from mutations that cause loss of the membrane-associated structural protein, dystrophin, and lead to a mechanically-weaker muscle cell membrane^{60,61}, most muscle fiber damage results from lysis caused by myeloid cells, especially macrophages expressing inducible nitric oxide synthase (iNOS) that are biased toward the M1, pro-inflammatory phenotype^{7,26}. However, as the disease progresses, macrophages in dystrophic muscle shift to a CD163+/CD206+ phenotype that increases muscle fibrosis²³ and is characteristic of type 2 immunity; much of the lethality of DMD is attributable to fibrosis of cardiac and respiratory muscles. Thus, by modulating the numbers and phenotype of macrophages in dystrophic muscle, LIF can produce broad effects on muscle pathology.

Some of the immunomodulatory effects achieved by transplantation of CD11b/LIF transgenic cells reflect the effects of transgene expression within the diseased muscle. For example, Socs3 expression was significantly elevated in muscles of mice that received CD11b/LIF BMT, although expression of the transgene in macrophages in vitro did not affect the expression of Socs3. LIF can increase Socs3 expression in multiple cell types⁶² and elevated expression or activity of Socs3 in macrophages can strongly influence their phenotype and cytokine production. In vivo models of inflammation show that siRNA-silencing of SOCS3 or targeted deletion of SOCS3 in macrophages can either promote⁶³ or oppose⁶⁴ the M1-biased phenotype. In experimental peritonitis, SOCS3 mRNA silencing in macrophages caused elevated expression of the M2 phenotypic markers Il10, Mrc1 and Arg1⁶⁴, which is consistent with the inverse relationship observed between elevated Socs3 expression in CD11b/LIF BMT recipients and reduced expression of Il10, Arg2 and Mrc1. Together, these observations suggest that the shift of CD11b/LIF macrophages away from an M2-biased phenotype in mdx BMT recipients may result, in part, from LIF induction of Socs3 after the transgenic macrophages enter the diseased muscle. However, some of the treatment effects observed may have resulted from immunomodulatory roles of the transgene that occurred before their invasion into the pathological muscle. The finding that isolated BMCs from CD11b/LIF mice showed greatly reduced levels of Cd163 and Arg1 expression as they differentiated to macrophages in vitro shows that some autocrine influences of the transgene on macrophage gene expression do not require localization of the cells in the dystrophic muscle. This contrasts with the reduced expression of TGFβ in intramuscular macrophages of CD11b/LIF BMT recipients that did not occur in transgenic macrophages in vitro. The reduction in arginase expression in CD11b/LIF transgenic macrophages may be particularly important in the pathophysiology of muscular dystrophy because arginine metabolism by arginase increases proline production which is necessary for collagen synthesis and contributes significantly to increased fibrosis in mdx muscles during progressive stages of pathology²³.

The immunomodulatory influences of the transgene extend beyond autocrine effects on macrophage phenotype, because the muscles of CD11b/LIF BMC recipients showed large reductions in the expression of ligands for CCR2. Previous investigators established that signaling through CCR2 is a primary mechanism for recruiting macrophages to diseased or injured muscle by showing that blockade or deletion of CCR2 greatly reduces macrophage entry into injured muscle^{27,45,47}. It was found that CD11b/LIF BMT decreased expression of CCR2 ligands in muscle and reduced the numbers of macrophages that expressed CCL2. Those reductions were also associated with large reductions in total numbers of F4/80+ intramuscular macrophages, including CD206+ and CD163+ macrophages. Thus, much of the anti-inflammatory effect of the transgene may occur through disruption of CCR2-mediated signaling, leading to reduced numbers of intramuscular macrophages and impairing their activation to a pro-fibrotic, M2-biased phenotype.

Collectively, the findings show that expression of a CD11b/LIF transgene in BMDCs can disrupt multiple processes that contribute to fibrosis of dystrophic muscle, including affecting macrophage recruitment, phenotype and production of pro-fibrotic cytokines and enzymes, in addition to preventing the fibrogenic conversion of satellite cells and reducing numbers of FAPs (FIG. 16). However, without wishing to be bound by theory, it is believed that the more broadly-significant finding in this investigation is that the data show that genetically-modified BMCs can be used as vectors to deliver therapeutic genes to dystrophic muscle. This approach is applicable not only to LIF, but may provide a more specific targeting strategy for the numerous gene products that have been previously identified as potentially-useful, therapeutic molecules for DMD.

### REFERENCES

1. Nelson, C. A. et al. Inhibiting TGF-β activity improves respiratory function in mdx mice. Am. J. Pathol. 178, 2611-2621 (2011).
2. Andreetta, F. et al. Immunomodulation of TGF-beta 1 in mdx mouse inhibits connective tissue proliferation in diaphragm but increases inflammatory response: implications for antifibrotic therapy. J. Neuroimmunol. 175, 77-86 (2006).
3. Taniguti, A. P. T., Pertille, A., Matsumura, C. Y., Santo Neto, H. & Marques. M. J. Prevention of muscle fibrosis and myonecrosis in mdx mice by suramin, a TGF-β1 blocker. Muscle Nerve 43, 82-87 (2011).
4. Bogdanovich, S. et al. Functional improvement of dystrophic muscle by myostatin blockade. Nature 420, 418-421 (2002).
5. Webling-Henricks, M. et al. Klotho gene silencing promotes pathology in the mdx mouse model of Duchenne muscular dystrophy. Hum. Mol. Genet. 25, 2465-2482 (2016).
6. Straub, V., Rafael. J. A., Chamberlain. J. S. & Campbell, K. P. Animal models for muscular dystrophy show different patterns of sarcolemmal disruption. J. Cell Biol. 139, 375-385 (1997).
7. Wehling, M., Spencer, M. J. & Tidball, J. G. A nitric oxide synthase transgene ameliorates muscular dystrophy in mdx mice. J. Cell Biol. 155, 123-131 (2001).
8. Spencer, M. J., Walsh, C. M., Dorshkind, K. A., Rodriguez, E. M. & Tidball, J. G. Myonuclear apoptosis in dystrophic mdx muscle occurs by perforin-mediated cytotoxicity. J. Clin. Invest. 99, 2745-2751 (1997).
9. Spencer. M. J., Montecino-Rodriguez, E., Dorshkind, K. & Tidball, J. G. Helper (CD4(+)) and cytotoxic (CD8(+)) T cells promote the pathology of dystrophin-deficient muscle. Clin. Immunol. 98, 235-243 (2001).
10. Cai. B., Spencer. M. J., Nakamura, G., Tseng-Ong. L. & Tidball, J. G. Eosinophilia of dystrophin-deficient muscle is promoted by perforin-mediated cytotoxicity by T cell effectors. Am. J. Pathol. 156, 1789-1796 (2000).
11. Hodgetts, S., Radley, H., Davies, M. & Grounds, M. D. Reduced necrosis of dystrophic muscle by depletion of host neutrophils, or blocking TNFalpha function with Etanercept in mdx mice. Neuromuscul. Disord. 16, 591-602 (2006).
12. Gorospe, J. R., Nishikawa. B. K. & Hoffman, E. P. Recruitment of mast cells to muscle after mild damage. J. Neurol. Sci. 135, 10-17 (1996).
13. Villalta, S. A., Deng, B., Rinaldi, C., Wehling-Henricks, M. & Tidball, J. G. IFN-γ promotes muscle damage in the mdx mouse model of Duchenne muscular dystrophy by suppressing M2 macrophage activation and inhibiting muscle cell proliferation. J. Immunol. 187, 5419-5428 (2011).
14. Villalta. S. A. et al. Interleukin-10 reduces the pathology of mdx muscular dystrophy by deactivating M1 macrophages and modulating macrophage phenotype. Hum. Mol. Genet. 20, 790-805 (2011).
15. Todd, R. F., Nadler, L. M. & Schlossman, S. F. Antigens on human monocytes identified by monoclonal antibodies. J. Immunol. 126, 1435-1442 (1981).
16. Griffin. J. D., Ritz, J., Nadler. L. M. & Schlossman. S. F. Expression of myeloid differentiation antigens on normal and malignant myeloid cells. J. Clin. Invest. 68, 932-941 (1981).
17. Rosmarin, A. G. et al. Differential expression of CD11b/CD18 (Mol) and myeloperoxidase genes during myeloid differentiation. Blood 73, 131-136 (1989).
18. Robertson, T. A., Maley, M. A., Grounds, M. D. & Papadimitriou, J. M. The role of macrophages in skeletal muscle regeneration with particular reference to chemotaxis. Exp. Cell Res. 207, 321-331 (1993).
19. Kurek. J., Bower, J., Romanella, M. & Austin, L. Leukaemia inhibitory factor treatment stimulates muscle regeneration in the mdx mouse. Neuroscience Letters 212, 167-170 (1996).
20. Austin. L. et al. Leukemia inhibitory factor ameliorates muscle fiber degeneration in the mdx mouse. Muscle Nerve 23, 1700-1705 (2000).
21. Hunt. L. C., Upadhyay, A., Jazayeri, J. A., Tudor. E. M. & White, J. D. An anti-inflammatory role for leukemia inhibitory factor receptor signaling in regenerating skeletal muscle. Histochem. Cell Biol. 139, 13-34 (2013).
22. Sica, A. & Mantovani, A. Macrophage plasticity and polarization: in vivo veritas. J. Clin. Invest. 122, 787-795 (2012).
23. Wehling-Henricks, M. et al. Arginine metabolism by macrophages promotes cardiac and muscle fibrosis in mdx muscular dystrophy. PLoS ONE 5, e10763 (2010).
24. Wehling-Henricks, M. et al. Macrophages escape Klotho gene silencing in the mdx mouse model of Duchenne muscular dystrophy and promote muscle growth and increase satellite cell numbers through a Klotho-mediated pathway. Hum. Mol. Genet. 27, 14-29 (2018).
25. Adams, T. E. et al. Growth hormone preferentially induces the rapid, transient expression of SOCS-3, a novel inhibitor of cytokine receptor signaling. J. Biol. Chem. 273, 1285-1287 (1998).
26. Villalta. S. A., Nguyen. H. X., Deng. B., Gotoh, T. & Tidball, J. G. Shifts in macrophage phenotypes and macrophage competition for arginine metabolism affect the severity of muscle pathology in muscular dystrophy. Hum. Mol. Genet. 18, 482-496 (2009).
27. Mojumdar, K. et al. Inflammatory monocytes promote progression of Duchenne muscular dystrophy and can be therapeutically targeted via CCR2. EMBO Mol Med 6, 1476-1492 (2014).
28. Curran, J. N., Winter, D. C. & Bouchier-Hayes, D. Biological fate and clinical implications of arginine metabolism in tissue healing. Wound Repair Regen 14, 376-386 (2006).
29. Vidal, B. et al. Fibrinogen drives dystrophic muscle fibrosis via a TGFbeta/alternative macrophage activation pathway. Genes Dev. 22, 1747-1752 (2008).
30. Zhou, L. et al. Temporal and spatial mRNA expression patterns of TGF-beta1, 2, 3 and TbetaRI, II, III in skeletal muscles of md mice. Neuromuscul. Disord. 16, 32-38 (2006).
31. Lemos, D. R. et al. Nilotinib reduces muscle fibrosis in chronic muscle injury by promoting TNF-mediated apoptosis of fibro/adipogenic progenitors. Nat. Med. 21, 786-794 (2015).
32. Uezumi, A. et al. Fibrosis and adipogenesis originate from a common mesenchymal progenitor in skeletal muscle. J. Cell. Sci. 124, 3654-3664 (2011).
33. Biressi, S., Miyabara, E. H., Gopinath, S. D., Carlig. P. M. M. & Rando, T. A. A Wnt-TGFβ2 axis induces a fibrogenic program in muscle stem cells from dystrophic mice. Sci Transl Med 6, 267ra176 (2014).
34. Peinado, H., Quintanilla, M. & Cano, A. Transforming growth factor beta-1 induces snai1 transcription factor in epithelial cell lines: mechanisms for epithelial mesenchymal transitions. J. Biol. Chem. 278, 21113-21123 (2003).
35. Liu, Y. et al. Transforming growth factor-β (TGF-β)-mediated connective tissue growth factor (CTGF) expression in hepatic stellate cells requires Stat3 signaling activation. J Biol Chem 288, 30708-30719 (2013).
36. Ignotz, R. A. & Massagué, J. Transforming growth factor-beta stimulates the expression of fibronectin and collagen and their incorporation into the extracellular matrix. J. Biol. Chem. 261, 4337-4345 (1986).
37. Lustig, B. et al. Negative feedback loop of Wnt signaling through upregulation of Conductin/Axin2 in colorectal and liver tumors. Mol. Cell. Biol. 22, 1184-1193 (2002).
38. Ishida, Y. & Nagata, K. Hsp47 as a collagen-specific molecular chaperone. Meth. Enzymol. 499, 167-182 (2011).
39. Taguchi, T., Nazneen, A., Al-Shihri, A. A., Turkistani, K. A. & Razzaque, M. S. Heat shock protein 47: a novel biomarker of phenotypically altered collagen-producing cells. Acta Histochem Cytochem 44, 35-41 (2011).
40. Brack, A. S. et al. Increased Wnt signaling during aging alters muscle stem cell fate and increases fibrosis. Science 317, 807-810 (2007).
41. Joe. A. W. B. et al. Muscle injury activates resident fibro/adipogenic progenitors that facilitate myogenesis. Nat. Cell Biol. 12, 153-163 (2010).
42. Uezumi, A., Fukada, S., Yamamoto, N., Takeda, S. & Tsuchida, K. Mesenchymal progenitors distinct from satellite cells contribute to ectopic fat cell formation in skeletal muscle. Nat. Cell Biol. 12, 143-152 (2010).
43. Mueller, A. A., van Velthoven, C. T., Fukumoto, K. D., Cheung, T. H. & Rando, T. A. Intronic polyadenylation of PDGFRα in resident stem cells attenuates muscle fibrosis. Nature 540, 276-279 (2016).
44. Tidball, J. G. & Wehling-Henricks. M. Macrophages promote muscle membrane repair and muscle fibre growth and regeneration during modified muscle loading in mice in vivo. J Physiol 578, 327-336 (2007).
45. Shireman, P. K. et al. MCP-1 deficiency causes altered inflammation with impaired skeletal muscle regeneration. J Leukoc Biol 81, 775-785 (2007).
46. Segawa, M. et al. Suppression of macrophage functions impairs skeletal muscle regeneration with severe fibrosis. Exp. Cell Res. 314, 3232-3244 (2008).
47. Martinez. C. O. et al. Regulation of skeletal muscle regeneration by CCR2-activating chemokines is directly related to macrophage recruitment. Am. J. Physiol. Regul. Integr. Comp. Physiol. 299, R832-842 (2010).
48. Ball, L. M. et al. Paediatric allogeneic bone marrow transplantation for homozygous beta-thalassemia, the Dutch experience. Bone Marrow Transplant. 31, 1081-1087 (2003).
49. Reuter, U. et al. Correction of complete interferon-gamma receptor 1 deficiency by bone marrow transplantation. Blood 100, 4234-4235 (2002).
50. Metcalf. D. & Gearing. D. P. Fatal syndrome in mice engrafted with cells producing high levels of the leukemia inhibitory factor. Proc. Natl. Acad. Sci. U.S.A. 86, 5948-5952 (1989).
51. Metcalf. D. & Gearing. D. P. A myelosclerotic syndrome in mice engrafted with cells producing high levels of leukemia inhibitory factor (LIF). Leukemia 3, 847-852 (1989).
52. White, J. D., Davies, M., McGeachie, J. & Grounds, M. D. An evaluation of leukaemia inhibitory factor as a potential therapeutic agent in the treatment of muscle disease. Neuromuscul. Disord. 12, 909-916 (2002).
53. Igarashi, A., Okochi, H., Bradham, D. M. & Grotendorst, G. R. Regulation of connective tissue growth factor gene expression in human skin fibroblasts and during wound repair. Mol. Biol. Cell 4, 637-645 (1993).
54. Frazier, K., Williams, S., Kothapalli, D., Klapper, H. & Grotendorst. G. R. Stimulation of fibroblast cell growth, matrix production, and granulation tissue formation by connective tissue growth factor. J. Invest. Dermatol. 107, 404-411 (1996).
55. Zanotti, S., Gibertini, S. & Mora. M. Altered production of extra-cellular matrix components by muscle-derived Duchenne muscular dystrophy fibroblasts before and after TGF-beta1 treatment. Cell Tissue Res. 339, 397-410 (2010).
56. Cencetti, F., Bernacchioni, C., Nincheri, P., Donati, C. & Bruni, P. Transforming growth factor-beta1 induces transdifferentiation of myoblasts into myofibroblasts via up-regulation of sphingosine kinase-1/S1P3 axis. Mol. Biol. Cell 21, 1111-1124 (2010).
57. Morales, M. G. et al. Reducing CTGF/CCN2 slows down mdx muscle dystrophy and improves cell therapy. Hum. Mol. Genet. 22, 4938-4951 (2013).
58. Gosselin, L. E. et al. Localization and early time course of TGF-beta 1 mRNA expression in dystrophic muscle. Muscle Nerve 30, 645-653 (2004).
59. Cohn, R. D. et al. Angiotensin II type 1 receptor blockade attenuates TGF-β-induced failure of muscle regeneration in multiple myopathic states. Nat Med 13, 204-210 (2007).
60. Hoffman, E. P., Brown, R. H. & Kunkel, L. M. Dystrophin: the protein product of the Duchenne muscular dystrophy locus. Cell 51, 919-928 (1987).
61. Petrof, B. J., Shrager, J. B., Stedman, H. H., Kelly, A. M. & Sweeney, H. L. Dystrophin protects the sarcolemma from stresses developed during muscle contraction. Proc. Natl. Acad. Sci. U.S.A. 90, 3710-3714 (1993).
62. Laszlo. G. S. & Nathanson, N. M. Src family kinase-independent signal transduction and gene induction by leukemia inhibitory factor. J. Biol. Chem. 278, 27750-27757 (2003).
63. Qin, H. et al. SOCS3 deficiency promotes M1 macrophage polarization and inflammation. J Immunol 189, 3439-3448 (2012).
64. Arnold, C. E. et al. A critical role for suppressor of cytokine signalling 3 in promoting M1 macrophage activation and function in vitro and in vivo. Immunology 141, 96-110(2014).
65. Amalfitano, A. & Chamberlain, J. S. The mdx-amplification-resistant mutation system assay, a simple and rapid polymerase chain reaction-based detection of the mdx allele. Muscle Nerve 19, 1549-1553 (1996).
66. Nolan, T., Hands, R. E. & Bustin, S. A. Quantification of mRNA using real-time RT-PCR. Nat Protoc 1, 1559-1582 (2006).
67. Bustin, S. A. et al. The MIQE guidelines: minimum information for publication of quantitative real-time PCR experiments. Clin. Chem. 55, 611-622 (2009).
68. Wang, Y., Wehling-Henricks, M., Samengo, G. & Tidball, J. G. Increases of M2a macrophages and fibrosis in aging muscle are influenced by bone marrow aging and negatively regulated by muscle-derived nitric oxide. Aging Cell 14, 678-688 (2015).
69. Burkholder, T. J., Fingado, B., Baron, S. & Lieber, R. L. Relationship between muscle fiber types and sizes and muscle architectural properties in the mouse hindlimb. J. Morphol. 221, 177-190 (1994).
70. Bellardita, C. & Kiehn, O. Phenotypic characterization of speed-associated gait changes in mice reveals modular organization of locomotor networks. Curr. Biol. 25, 1426-1436 (2015).

### SEQUENCES

All polypeptide sequences are presented N-terminal to C-terminal unless otherwise noted.

All nucleic acid sequences are presented 5′ to 3′ unless otherwise noted.

Nucleic acid sequence of Homo sapiens
CD11b promoter (SEQ ID NO: 1):
TGCAGCCTGGACCTCGGGCTCAAGTAATTCTCACACCTCA
GCCTGTCCAGTAGCAGGGGCTACAGGCGCGCACCACCATG
CCCAGCTAATTAAAAATATTTTTTTGTAGAGACAGGGTCT
CTCTATGTTGCCCAGGCTGGTTTCAAACTCCCAGGCTCAA
GCAATCCTCCTGCCTTGGCCTCCCAAAGTGCTGGCATTAC
AGGCGTGAGCCACTGCGCCTGGCCCGTATTAATGTTTAGA
ACACGAATTCCAGGAGGCAGGCTAAGTCTGTTCAGCTTGT
TCATATGCTTGGGCCAACCCAAGAAACAAGTGGGTGACAA
ATGGCACCTTTTGGATAGTGGTATTGACTTTGAAAGTTTG
GGTCAGGAAGCTGGGGAGGAAGGGTGGGCAGGCTGTGGGC
AGTCCTGGGCGGAAGACCAGGCAGGGCTATGTGCTCACTG
AGCCTCCGCCCTCTTCCTTTGAATCTCTGATAGACTTCTG
CCTCCTACTTCTCCTTTTCTGCCCTTCTTTGCTTTGGTGG
CTTCCTTGTGGTTCCTCAGTGGTGCCTGCA
Nucleic acid sequence of Pan troglodytes
CD11b promoter (SEQ ID NO: 2):
TGCAGCCTCCACTTCCGAGGCTCAAGCAATCCTCCTGCCT
CAGCCTCCCCATAGCAGGGACCACAGGCACACACCACCAC
ACCCGGCTAATTTTAAATTTTGAGAGAGGGATCTTGCTAT
GTTGCCCAGGCTGGTTCAAACTCCTGGGTTCAAGCAATCC
TCCTGCCTTGGCCTCCCAAAGTGCTGGGATTACAGGAGTG
AGCCACT
Nucleic acid sequence of Homo sapiens
LIF polypeptide (SEQ ID NO: 3):
ATGAAGGTCTTGGCGGCAGGAGTTGTGCCCCTGCTGTTGG
TTCTGCACTGGAAACATGGGGCGGGGAGCCCCCTCCCCAT
CACCCCTGTCAACGCCACCTGTGCCATACGCCACCCATGT
CACAACAACCTCATGAACCAGATCAGGAGCCAACTGGCAC
AGCTCAATGGCAGTGCCAATGCCCTCTTTATTCTCTATTA
CACAGCCCAGGGGGAGCCGTTCCCCAACAACCTGGACAAG
CTATGTGGCCCCAACGTGACGGACTTCCCGCCCTTCCACG
CCAACGGCACGGAGAAGGCCAAGCTGGTGGAGCTGTACCG
CATAGTCGTGTACCTTGGCACCTCCCTGGGCAACATCACC
CGGGACCAGAAGATCCTCAACCCCAGTGCCCTCAGCCTCC
ACAGCAAGCTCAACGCCACCGCCGACATCCTGCGAGGCCT
CCTTAGCAACGTGCTGTGCCGCCTGTGCAGCAAGTACCAC
GTGGGCCATGTGGACGTGACCTACGGCCCTGACACCTCGG
GTAAGGATGTCTTCCAGAAGAAGAAGCTGGGCTGTCAACT
CCTGGGGAAGTATAAGCAGATCATCGCCGTGTTGGCCCAG
GCCTTCTAG
Nucleic acid sequence of Mus musculus
LIF polypeptide (SEQ ID NO: 4):
ATGAAGGTCTTGGCCGCAGGGATTGTGCCCTTACTGCTGC
TGGTTCTGCACTGGAAACACGGGGCAGGGAGCCCTCTTCC
CATCACCCCTGTAAATGCCACCTGTGCCATACGCCACCCA
TGCCACGGCAACCTCATGAACCAGATCAAGAATCAACTGG
CACAGCTCAATGGCAGCGCCAATGCTCTCTTCATTTCCTA
TTACACAGCTCAAGGGGAGCCGTTTCCCAACAACGTGGAA
AAGCTATGTGCGCCTAACATGACAGACTTCCCATCTTTCC
ATGGCAACGGGACAGAGAAGACCAAGTTGGTGGAGCTGTA
TCGGATGGTCGCATACCTGAGCGCCTCCCTGACCAATATC
ACCCGGGACCAGAAGGTCCTGAACCCCACTGCCGTGAGCC
TCCAGGTCAAGCTCAATGCTACTATAGACGTCATGAGGGG
CCTCCTCAGCAATGTGCTTTGCCGTCTGTGCAACAAGTAC
CGTGTGGGCCACGTGGATGTGCCACCTGTCCCCGACCACT
CTGACAAAGAAGCCTTCCAAAGGAAAAAGTTGGGTTGCCA
GCTTCTGGGGACATACAAGCAAGTCATAAGTGTGGTGGTC
CAGGCCTTCTAG
Nucleic acid sequence of Rattus norvegicus
LIF polypeptide (SEQ ID NO: 5):
ATGAAGGTCTTGGCCACAGGGATTGTGCCCCTACTGCTCA
TTCTGGCACTGGAAACACGGGGCAGGGAGCCCCCTTCCCA
TCACCCCTGTAAATGCCACCTGCGCCATACGCCACCCGTG
TCACGGCAACCTCATGAACCAGATCAAGAGTCAACTGGCT
CAACTCAACGGCAGTGCCAATGCCCTCTTTATTTCCTATT
ACACAGCTCAAGGGGAACCATTTCCCAACAACGTGGATAA
GCTATGTGCGCCCAACATGACGGATTTCCCACCTTTCCAT
GCCAATGGGACAGAGAAGACCAAGTTGGTCGAGCTGTATC
GGATGGTCGCGTACCTGGGAGCCTCCCTGACCAACATCAC
CTGGGATCAGAAAAACCTCAACCCCACTGCCGTGAGCCTC
CAGATCAAACTCAATGCGACTACAGACGTCATGAGGGGGC
TCCTTAGCAACGTGCTTTGCCGTCTGTGCAACAAGTACCA
TGTGGGCCATGTGGATGTGCCCTGTGTCCCCGACAACTCT
AGCAAAGAAGCCTTCCAAAGGAAGAAGTTGGGCTGCCAGC
TCCTGGGGACATACAAGCAAGTCATTAGTGCGGTGGTCCA
GGCCTTCTAG
Nucleic acid sequence of Canis lupus familiaris
LIF polypeptide (SEQ ID NO: 6):
ATGAAGGTCTTGGCGGCAGGAGTTGTGCCCCTGCTGCTGG
TTCTGCACTGGAAACACGGGGCAGGGAGCCCCCTTCCTAT
CACCCCTGTCAACGCCACCTGCGCCACACGCCACCCATGT
CACAGCAACCTCATGAACCAGATCAGGAACCAACTGGCGC
AGCTCAATGGCAGCGCCAATGCTCCTCTTTATTCTCTATT
ACACAGCCCAGGGGGAGCTCGTTCCCCAACAACCTGGACA
AGCTGTGTGGCCCCAACGTGACAGACTTCCCACCATTCCA
CGCCAACAGCACGGAGAAGACCCGGCTGGTGGAGCTGTAC
CGCATCATCGCCTACCTTGGGGCCTCCCTGGGCAACATCA
CCCGGGACCAGAAGGTCCTCAATCCCAATGCCCTCAGCCT
CCACAGCAAGCTGAATGCCACTGCGGACATCATGCGGGGC
CTCCTCAGCAACGTGCTCTGCCGCCTGTGTAGCAAGTACC
ACGTGGCCCACGTGGACGTGGCCTATGGGCCCGACACCTC
GGGCAAGGATGTCTTCCAGAAGAAGAAGTTGGGCTGTCAG
CTCCTGGGGAAGTATAAGCAGGTCATTGCCGTGGTGGCCC
AGGCCTTCTAG
Nucleic acid sequence of Meriones unguiculatus
LIF polypeptide (SEQ ID NO: 7):
ATGAAGGTCTTGGCCGCAGGGGTTGTGCCCTTACTGCTGG
TTCTGCACTGGAAACACGGGGCTGGGAGCCCCCTTCCCAT
CACCCCCGTTAATGCCACCTGTGCCACACGCCACCCATGT
CATGGCAACCTCATGCACCAGATTAAGAATCAACTGGCTC
AGCTCAATGGCAGTGCCAATGCCCTCTTCATCTCCTATTA
CACAGCTCAAGGGGAGCCATTTCCCAACAACCTGGACAAG
CTGTGTGGGCCTAACATGACAGATTTCCCGCCTTTCCACG
CCAACGGGACGGAGAAGAGCAAGTTGGTGGAGCTGTATCG
GATGGTCGCGTACCTGGGTGCCTCCCTGGGCAACGTCACC
CGGGATCAGAAGGTCCTCAACCCCAACGCCCTGAGCCTCC
ACGGCAAACTCAACGCCACTATCGACGTCATGCGCGGCCT
CCTCAACAATGTGCTCTGTCGCCTGTGCAACAAGTACCAC
GTCGGCCACGTGGACGTGACGTACGCCCCCGACACCTCCA
GCAAAGAGATCTTCCAAAAGAAAAAGGCGGGCTGCCAGCT
CCTAGGGACGTACAAGCAAGTCATTAGCGTGGTGGCCCAG
GCCTTCTAG
Nucleic acid sequence of Ailuropoda melanoleuca
LIF polypeptide (SEQ ID NO: 8):
ATGAAGGTCTTGGCGGCAGGAGTTGTGCCCCTGCTGCTGG
TTCTGCACTGGAAACACGGGGCGGGGAGCCCCCTTCCTAT
CACCCCTGTCAACGCCACCTGCGCCACACGCCACCCATGT
CACAGCAACCTCATGAACCAGATCAGGAACCAACTGGCGC
AGCTCAATGGCAGTGCCAATGCTCTCTTTATTCTCTATTA
CACAGCCCAGGGGGAGCCGTTCCCCAACAACCTGGACAAG
CTGTGCGGCCCCAACGTGACGGACTTCCCGCCATTCCACG
CCAACGGCACGGAGAGGACGCGGCTCGTGGAGTTGTACCG
CCTCATCGCGTACCTCGGCGCCTCCCTGGGCAACATCACC
CGGGACCAGAAGGTCCTCAATCCCAACGCCCTCAGCCTCC
ACAGCAAGCTGAACGCCACTGCGGACATCATGCGGGGCCT
CCTCAGCAACGTGCTCTGCCGCCTGTGTAACAAGTACCAC
GTGGCCCACGTGGACGTGGCCTACGGCCCCGACACCTCGG
GCAAGGACGTCTTCCAGAAGAAGAAGTTGGGCTGTCAGCT
CCTGGGGAAGTATAAGCAGGTCATCGCCGTGGTGGCCCAG
GCCTTCTAG
Nucleic acid sequence of Odobenus rosmarus
divergens LIF polypeptide (SEQ ID NO: 9):
ATGAAGGTCTTGGCGGCAGGAGTTGTGCCCCTGCTGCTGG
TTCTGCACTGGAAACACGGGGCGGGGAGCCCCCTTCCTAT
CACCCCTGTCAACGCCACCTGTGCCACACGCCACCCATGT
CACAGCAACCTCATGAACCAGATCAGGAACCAACTGGCGC
AGCTCAATGGCAGTGCCAATGCTCTCTTTATTCTCTATTA
CACAGCCCAGGGGGAGCCGTTCCCCAACAACCTGGACAAG
CTGTGTGGCCCCAATGTGACGGACTTCCCGCCATTCCACG
CCAACGGCACGGAGAAGACGCGGCTAGTGGAGCTGTACCG
CATCATCGCGTACCTCGGCGCCTCCCTGGGCAACATCACC
CGGGACCAGAAGGTCCTCAATCCCAACGCCCTCAGCCTCC
ATAGCAAGCTGAATGCCACTGCGGACATCATGCGGGGCCT
CCTTAGCAACGTGCTCTGCCGCCTGTGTAACAAGTACCAC
GTGGCCCACGTGGACGTGGCCTACGGCCCCGACACCTCGG
GCAAGGACGTCTTCCAGAAGAAGAAGTTGGGCTGTCAGCT
CCTGGGGAAGTATAAGCAGGTCATCGCTGTGGTGGCTCAG
GCCTTCTAG
Nucleic acid sequence of Pteropus vampyrus
LIF polypeptide (SEQ ID NO: 10):
ATGAAGGTCTTGGCAGCAGGAGTCGTCCCCCTGCTGCTGG
TTCTGCACTGGAAACACGGGGCAGGGAGCCCCCTTCCCAT
CACCCCTGTCAACGCCACCTGTGTCACACGCCACCCATGT
CACAACAACCTCATGAACCAGATCAGGAACCAACTGGCCC
AGCTCAACAGCAGTGCCAATGCCCTCTTCATTCTCTATTA
CACGGCCCAAGGGGAGCCATTCCCCAACAACCTGGACAAG
CTGTGTGGCCCCAACGTGACAGACTTCCCACCCTTCCATG
CCAATGGCACGGAAAAGGCCCGGCTGGTGGAGCTGTACCG
CATCATCGCGTACCTTGGCGCCTCCCTGGGCAACATCACA
CGGGACCAGAAGATCCTCAATCCCAACGCCCTCAGCCTCC
ACAGCAAGCTGAATGCCACCACAGACACCATGCGGGGCCT
CCTCAGTAACGTGCTATGCCGCCTGTGCAGCAAGTACCAC
GTGGCCCACGTGGAGGTGGCCTATGGCCCTGACACCTCAG
GCAAGGATGTCTTCCAGAAGAAGAAATTGGGCTGTCAGCT
CCTGGGGAAGTATAAGCAGGTCATCGCTGTAGTGGCCCAG
GCCTTCTAG
Nucleic acid sequence of Nannospalax galili
LIF polypeptide (SEQ ID NO: 11):
ATGAAGGTCTTGGCCGCAGGAGTTGTGCCCTTGCTGCTGG
TTCTGCACTGGAAACACGGGGCAGCGAGCCCCCTTCCCAT
CACCCCTGTTAATGCTACCTGTGCCACACGCCACCCATGT
CATGGCAACCTCATGAATCAGATCAAGAACCAATTGGCTC
AGCTCAATGGCAGTGCCAATGCCCTCTTCATTTCCTATTA
TACAGCTCAGGGGGAGCCATTCCCCAACAACCTCGACAAG
CTGTGTGGACCCAACATGACGGACTTCCCACCCTTCCATG
CCAATGGCACAGAAAAGGCCAAGCTGGTGGAGCTGTATCG
TTTGGTCGCATACCTGGGTGCCTCCCTGGGCAACGTCACC
CGGGATCAGAAGATCCTGAACCCCAATGCCCTGAGCCTCC
ACAGCAAGCTCAATGCCACCACAGACACCATGCGGGGCCT
CCTCAGCAATGTGCTTTGTCGCCTGTGCAACAAGTACCAT
GTGGGCCACGTGGACGTGACCTATGGCCCTGACACCGCAG
GCAAGGATGCTTTCCAAAAGAAAAAGCTGGGCTGCCAGCT
CCTGGGGACGTACAAGCAAGTCATTAGCCTGGTGGCCCAG
GCCTTCTAG
Nucleic acid sequence of Homo sapiens
CD1lb promoter + Miss musculus
LIF polypeptide (SEQ ID NO: 12):
TGCAGCCTGGACCTCGGGCTCAAGTAATTCTCACACCTCAG
CCTGTCCAGTAGCAGGGGCTACAGGCGCGCACCACCATGCC
CAGCTAATTAAAAATATTTTTTGTAGAGACAGCGGTC
TCTCTATGTTGCCCAGGCTGGTTTCAAACTCCCAGGCTCA
AGCAATCCTCCTGCCTTGGCCTCCCAAAGTGCTGGCATTA
CAGGCGTGAGCCACTGCGCCTGGCCCGTATTAATGTTTAG
AACACGAATTCCAGGAGGCAGGCTAAGTCTGTTCAGCTTG
TTCATATGCTTGGGCCAACCCAAGAAACAAGTGGGTGACA
AATGGCACCTTTTGGATAGTGGTATTGACTTTGAAAGTTT
GGGTCAGGAAGCTGGGGAGGAAGGGTGGGCAGGCTGTGGG
CAGTCCTGGGCGGAAGACCAGGCAGGGCTATGTGCTCACT
GAGCCTCCGCCCTCTTCCTTTGAATCTCTGATAGACTTCT
GCCTCCTACTTCTCCTTTTCTGCCCTTCTTTGCTTTGGTG
GCTTCCTTGTGGTTCCTCAGTGGTGCCTGCAAAGCTTGGC
ATTCCGGTACTGTTGGTAAAGCCACCATGTACATGAAGGT
CTTGGCCGCAGGGATTGTGCCCTTACTGCTGCTGGTTCTG
CACTGGAAACACGGGGCAGGGAGCCCTCTTCCCATCACCC
CTGTAAATGCCACCTGTGCCATACGCCACCCATGCCACGG
CAACCTCATGAACCAGATCAAGAATCAACTGGCACAGCTC
AATGGCAGCGCCAATGCTCTCTTCATTTCCTATTACACAG
CTCAAGGGGAGCCGTTTCCCAACAACGTGGAAAAGCTATG
TGCGCCTAACATGACAGACTTCCCATCTTTCCATGGCAAC
GGGACAGAGAAGACCAAGTTGGTGGAGCTGTATCGGATGG
TCGCATACCTGAGCGCCTCCCTGACCAATATCACCCGGGA
CCAGAAGGTCCTGAACCCCACTGCCGTGAGCCTCCAGGTC
AAGCTCAATGCTACTATAGACGTCATGAGGGGCCTCCTCA
GCAATGTGCTTTGCCGTCTGTGCAACAAGTACCGTGTGGG
CCACGTGGATGTGCCACCTGTCCCCGACCACTCTGACAAA
GAAGCCTTCCAAAGGAAAAAGTTGGGTTGCCAGCTTCTGG
GGACATACAAGCAAGTCATAAGTGTGGTGGTCCAGGCCTT
CTAG
Nucleic acid sequence of Homo sapiens
CD1 1b promoter + Homo sapiens
LIF polypeptide (SEQ ID NO: 13):
TGCAGCCTGGACCTCGGGCTCAAGTAATTCTCACACCTCA
GCCTGTCCAGTAGCAGGGGCTACAGGCGCGCACCACCATG
CCCAGCTAATTAAAAATATTTTTTTGTAGAGACAGGGTCT
CTCTATGTTGCCCAGGCTGGTTTCAAACTCCCAGGCTCAA
GCAATCCTCCTGCCTTGGCCTCCCAAAGTGCTGGCATTAC
AGGCGTGAGCCACTGCGCCTGGCCCGTATTAATGTTTAGA
ACACGAATTCCAGGAGGCAGGCTAAGTCTGTTCAGCTTGT
TCATATGCTTGGGCCAACCCAAGAAACAAGTGGGTGACAA
ATGGCACCTTTTGGATAGTGGTATTGACTTTGAAAGTTTG
GGTCAGGAAGCTGGGGAGGAAGGGTGGGCAGGCTGTGGGC
AGTCCTGGGCGGAAGACCAGGCAGGGCTATGTGCTCACTG
AGCCTCCGCCCTCTTCCTTTGAATCTCTGATAGACTTCTG
CCTCCTACTTCTCCTTTTCTGCCCTTCTTTGCTTTGGTGG
CTTCCTTGTGGTTCCTCAGTGGTGCCTGCAAAGCTTGGCA
TTCCGGTACTGTTGGTAAAGCCACCATGTACATGAAGGTC
TTGGCGGCAGGAGTTGTGCCCCTGCTGTTGGTTCTGCACT
GGAAACATGGGGCGGGGAGCCCCCTCCCCATCACCCCTGT
CAACGCCACCTGTGCCATACGCCACCCATGTCACAACAAC
CTCATGAACCAGATCAGGAGCCAACTGGCACAGCTCAATG
GCAGTGCCAATGCCCTCTTTATTCTCTATTACACAGCCCA
GGGGGAGCCGTTCCCCAACAACCTGGACAAGCTATGTGGC
CCCAACGTGACGGACTTCCCGCCCTTCCACGCCAACGGCA
CGGAGAAGGCCAAGCTGGTGGAGCTGTACCGCATAGTCGT
GTACCTTGGCACCTCCCTGGGCAACATCACCCGGGACCAG
AAGATCCTCAACCCCAGTGCCCTCAGCCTCCACAGCAAGC
TCAACGCCACCGCCGACATCCTGCGAGGCCTCCTTAGCAA
CGTGCTGTGCCGCCTGTGCAGCAAGTACCACGTGGGCCAT
GTGGACGTGACCTACGGCCCTGACACCTCGGGTAAGGATG
TCTTCCAGAAGAAGAAGCTGGGCTGTCAACTCCTGGGGAA
GTATAAGCAGATCATCGCCGTGTTGGCCCAGGCCTTCTAG
Nucleic acid sequence of Homo sapiens
Klotho polypeptide (SEQ ID NO: 14):
ATGCCCGCCAGCGCCCCGCCGCGCCGCCCGCGGCCGCCGC
CGCCGTCGCTGTCGCTGCTGCTGGTGCTGCTGGGCCTGGG
CGGCCGCCGCCTGCGTGCGGAGCCGGGCGACGGCGCGCAG
ACCTGGGCCCGTTTCTCGCGGCCTCCTGCCCCCGAGGCCG
CGGGCCTCTTCCAGGGCACCTTCCCCGACGGCTTCCTCTG
GGCCGTGGGCAGCGCCGCCTACCAGACCGAGGGCGGCTGG
CAGCAGCACGGCAAGGGTGCGTCCATCTGGGATACGTTCA
CCCACCACCCCCTGGCACCCCCGGGAGACTCCCGGAACGC
CAGTCTGCCGTTGGGCGCCCCGTCGCCGCTGCAGCCCGCC
ACCGGGGACGTAGCCAGCGACAGCTACAACAACGTCTTCC
GCGACACGGAGGCGCTGCGCGAGCTCGGGGTCACTCACTA
CCGCTTCTCCATCTCGTGGGCGCGAGTGCTCCCCAATGGC
AGCGCGGGCGTCCCCAACCGCGAGGGGCTGCGCTACTACC
GGCGCCTGCTGGAGCGGCTGCGGGAGCTGGGCGTGCAGCC
CGTGGTCACCCTGTACCACTGGGACCTGCCCCAGCGCCTG
CAGGACGCCTACGGCGGCTGGGCCAACCGCGCCCTGGCCG
ACCACTTCAGGGATTACGCGGAGCTCTGCTTCCGCCACTT
CGGCGGTCAGGTCAAGTACTGGATCACCATCGACAACCCC
TACGTGGTGGCCTGGCACGGCTACGCCACCGGGCGCCTGG
CCCCCGGCATCCGGGGCAGCCCGCGGCTCGGGTACCTGGT
GGCGCACAACCTCCTCCTGGCTCATGCCAAAGTCTGGCAT
CTCTACAATACTTCTTTCCGTCCCACTCAGGGAGGTCAGG
TGTCCATTGCCCTAAGCTCTCACTGGATCAATCCTCGAAG
AATGACCGACCACAGCATCAAAGAATGTCAAAAATCTCTG
GACTTTGTACTAGGTTGGTTTGCCAAACCCGTATTTATTG
ATGGTGACTATCCCGAGAGCATGAAGAATAACCTTTCATC
TATTCTGCCTGATTTTACTGAATCTGAGAAAAAGTTCATC
AAAGGAACTGCTGACTTTTTTGCTCMTTGCTTTGGACCCA
CCTTGAGTTTTCAACTTTTGGACCCTCACATGAAGTTCCG
CCAATTGGAATCTCCCAACCTGAGGCAACTGCTTTCCTGG
ATTGACCTTGAATTTAACCATCCTCAAATATTTATTGTGG
AAAATGGCTGGTTTGTCTCAGGGACCACCAAGAGAGATGA
TGCCAAATATATGTATTACCTCAAAAAGTTCATCATGGAA
ACCTTAAAAGCCATCAAGCTGGATGGGGTGGATGTCATCG
GGTATACCGCATGGTCCCTCATGGATGGTTTCGAGTGGCA
CAGAGGTTACAGCATCAGGCGTGGACTCTTCTATGTTGAC
TTTCTAAGCCAGGACAAGATGTTGTTGCCAAAGTCTTCAG
CCTTGTTCTACCAAAAGCTGATAGAGAAAAATGGCTTCCC
TCCTTTACCTGAAAATCAGCCCCTAGAAGGGACATTTCCC
TGTGACTTTGCTTGGGGAGTTGTTGACAACTACATTCAAG
TAGATACCACTCTGTCTCAGTTTACCGACCTGAATGTTTA
CCTGTGGGATGTCCACCACAGTAAAAGGCTTATTAAAGTG
GATGGGGTTGTGACCAAGAAGAGGAAATCCTACTGTGTTG
ACTTTGCTGCCATCCAGCCCCAGATCGCTTTACTCCAGGA
AATGCACGTTACACATTTTCGCTTCTCCCTGGACTGGGCC
CTGATTCTCCCTCTGGGTAACCAGTCCCAGGTGAACCACA
CCATCCTGCAGTACTATCGCTGCATGGCCAGCGAGCTTGT
CCGTGTCAACATCACCCCAGTGGTGGCCCTGTGGCAGCCT
ATGGCCCCGAACCAAGGACTGCCGCGCCTCCTGGCCAGGC
AGGGCGCCTGGGAGAACCCCTACACTGCCCTGGCCTTTGC
AGAGTATGCCCGACTGTGCTTTCAAGAGCTCGGCCATCAC
GTCAAGCTTTGGATAACGATGAATGAGCCGTATACAAGGA
ATATGACATACAGTGCTGGCCACAACCTTCTGAAGGCCCA
TGCCCTGGCTTGGCATGTGTACAATGAAAAGTTTAGGCAT
GCTCAGAATGGGAAAATATCCATAGCCTTGCAGGCTGATT
GGATAGAACCTGCCTGCCCTTTCTCCCAAAAGGACAAAGA
GGTGGCTGAGAGAGTTTTGGAATTTGACATTGGCTGGCTG
GCTGAGCCCATTTTCGGCTCTGGAGATTATCCATGGGTGA
TGAGGGACTGGCTGAACCAAAGAAACAATTTTCTTCTTCC
TTATTTCACTGAAGATGAAAAAAAGCTAATCCAGGGTACC
TTTGACTTTTTGGCTTTAAGCCATTATACCACCATCCTTG
TAGACTCAGAAAAAGAAGATCCAATAAAATACAATGATTA
CCTAGAAGTGCAAGAAATGACCGACATCACGTGGCTCAAC
TCCCCCAGTCAGGTGGCGGTAGTGCCCTGGGGGTTGCGCA
AAGTGCTGAACTGGCTGAAGTTCAAGTACGGAGACCTCCC
CATGTACATAATATCCAATGGAATCGATGACGGGCTGCAT
GCTGAGGACGACCAGCTGAGGGTGTATTATATGCAGAATT
ACATAAACGAAGCTCTCAAAGCCCACATACTGGATGGTAT
CAATCTTTGCGGATACTTTGCTTATTCGTTTAACGACCGC
ACAGCTCCGAGGTTTGGCCTCTATCGTTATGCTGCAGATC
AGTTTGAGCCCAAGGCATCCATGAAACATTACAGGAAAAT
TATTGACAGCAATGGTTTCCCGGGCCCAGAAACTCTGGAA
AGATTTTGTCCAGAAGAATTCACCGTGTGTACTGAGTGCA
GTTTTTTTCACACCCGAAAGTCTTTACTGGCTTTCATAGC
TTTTCTATTTTTTGCTTCTATTATTTCTCTCTCCCTTATA
TTTTACTACTCGAAGAAAGGCAGAAGAAGTTACAAATAG
Nucleic acid sequence of Mus musculus
Klotho polypeptide (SEQ ID NO: 15):
ATGCTAGCCCGCGCCCCTCCTCGCCGCCCGCCGCGGCTGG
TGCTGCTCCGTTTGCTGTTGCTGCATCTGCTGCTGCTCGC
CCTGCGCGCCCGCTGCCTGAGCGCTGAGCCGGGTCAGGGC
GCGCAGACCTGGGCTCGCTTCGCGCGCGCTCCTGCCCCAG
AGGCCGCTGGCCTCCTCCACGACACCTTCCCCGACGGTTT
CCTCTGGGCGGTAGGCAGCGCCGCCTATCAGACCGAGGGC
GGCTGGCGACAGCACGGCAAAGGCGCGTCCATCTGGGACA
CTTTCACCCATCACTCTGGGGCGGCCCCGTCCGACTCCCC
GATCGTCGTGGCGCCGTCGGGTGCCCCGTCGCCTCCCCTG
TCCTCCACTGGAGATGTGGCCAGCGATAGTTACAACAACG
TCTACCGCGACACAGAGGGGCTGCGCGAACTGGGGGTCAC
CCACTACCGCTTCTCCATATCGTGGGCGCGGGTGCTCCCC
AATGGCACCGCGGGCACTCCCAACCGCGAGGGGCTGCGCT
ACTACCGGCGGCTGCTGGAGCGGCTGCGGGAGCTGGGCGT
GCAGCCGGTGGTTACCCTGTACCATTGGGACCTGCCACAG
CGCCTGCAGGACACCTATGGCGGATGGGCCAATCGCGCCC
TGGCCGACCATTTCAGGGATTATGCCGAGCTCTGCTTCCG
CCACTTCGGTGGTCAGGTCAAGTACTGGATCACCATTGAC
AACCCCTACGTGGTGGCCTGGCACGGGTATGCCACCGGGC
GCCTGGCCCCGGGCGTGAGGGGCAGCTCCAGGCTCGGGTA
CCTGGTTGCCCACAACCTACTTTTGGCTCATGCCAAAGTC
TGGCATCTCTACAACACCTCTTTCCGCCCCACACAGGGAG
GCCGGGTGTCTATCGCCTTAAGCTCCCATTGGATCAATCC
TCGAAGAATGACTGACTATAATATCAGAGAATGCCAGAAG
TCTCTTGACTTTGTGCTAGGCTGGTTTGCCAAACCCATAT
TTATTGATGGCGACTACCCAGAGAGTATGAAGAACAACCT
CTCGTCTCTTCTGCCTGATTTTACTGAATCTGAGAAGAGG
CTCATCAGAGGAACTGCTGACTTTTTTGCTCTCTCCTTCG
GACCAACCTTGAGCTTTCAGCTATTGGACCCTAACATGAA
GTTCCGCCAATTGGAGTCTCCCAACCTGAGGCAGCTTCTG
TCTTGGATAGATCTGGAATATAACCACCCTCCAATATTTA
TTGTGGAAAATGGCTGGTTTGTCTCGGGAACCACCAAAAG
GGATGATGCCAAATATATGTATTATCTCAAGAAGTTCATA
ATGGAAACCTTAAAAGCAATCAGACTGGATGGGGTCGACG
TCATTGGGTACACCGCGTGGTCGCTCATGGACGGTTTCGA
GTGGCATAGGGGCTACAGCATCCGGCGAGGACTCTTCTAC
GTTGACTTTCTGAGTCAGGACAAGGAGCTGTTGCCAAAGT
CTTCGGCCTTGTTCTACCAAAAGCTGATAGAGGACAATGG
CTTTCCTCCTTTACCTGAAAACCAGCCCCTTGAAGGGACA
TTTCCCTGTGACTTTGCTTGGGGAGTTGTTGACAACTACG
TTCAAGTGGACACTACTCTCTCTCAGTTTACTGACCCGAA
TGTCTATCTGTGGGATGTGCATCACAGTAAGAGGCTTATT
AAAGTAGACGGGGTTGTAGCCAAGAAGAGAAAACCTTACT
GTGTTGATTTCTCTGCCATCCGGCCTCAGATAACCTTACT
TCGAGAAATGCGGGTCACCCACTTTCGCTTCTCCCTGGAC
TGGGCCCTGATCTTGCCTCTGGGTAACCAGACCCAAGTGA
ACCACACGGTTCTGCACTTCTACCGCTGCATGATCAGCGA
GCTGGTGCACGCCAACATCACTCCAGTGGTGGCCCTGTGG
CAGCCAGCAGCCCCGCACCAAGGCCTGCCACATGCCCTTG
CAAAACATGGGGCCTGGGAGAACCCGCACACTGCTCTGGC
GTTTGCAGACTACGCAAACCTGTGTTTTAAAGAGTTGGGT
CACTGGGTCAATCTCTGGATCACCATGAACGAGCCAAACA
CACGGAACATGACCTATCGTGCCGGGCACCACCTCCTGAG
AGCCCATGCCTTGGCTTGGCATCTGTACGATGACAAGTTT
AGGGCGGCTCAGAAAGGCAAAATATCCATCGCCTTGCAGG
CTGACTGGATAGAACCGGCCTGCCCTTTCTCTCAAAATGA
CAAAGAAGTGGCCGAGAGAGTTTTGGAATTTGATATAGGC
TGGCTGGCAGAGCCTATTTTTGGTTCCGGAGATTATCCAC
GTGTGATGAGGGACTGGCTGAACCAAAAAAACAATTTTCT
TTTGCCCTATTTCACCGAAGATGAAAAAAAGCTAGTCCGG
GGTTCCTTTGACTTCCTGGCGGTGAGTCATTACACCACCA
TTCTGGTAGACTGGGAAAAGGAGGATCCGATGAAATACAA
CGATTACTTGGAGGTACAGGAGATGACTGACATCACATGG
CTCAACTCTCCCAGTCAGGTGGCAGTGGTGCCTTGGGGGC
TGCGCAAAGTGCTCAACTGGCTAAGGTTCAAGTACGGAGA
CCTCCCGATGTATGTGACAGCCAATGGAATCGATGATGAC
CCCCACGCCGAGCAAGACTCACTGAGGATCTATTATATTA
AGAATTATGTGAATGAGGCTCTGAAAGCCTACGTGTTGGA
CGACATCAACCTTTGTGGCTACTTTGCGTATTCACTTAGT
GATCGCTCAGCTCCCAAGTCTGGCTTTTATCGATATGCTG
CGAATCAGTTTGAGCCCAAACCATCTATGAAACATTACAG
GAAAATTATTGACAGCAATGGCTTCCTGGGTTCTGGAACA
CTGGGAAGGTTTTGTCCAGAAGAATACACTGTGTGCACCG
AATGTGGATTTTTTCAAACCCGGAAGTCTTTGCTGGTCTT
CATCTCGTTTCTTGTTTTTACTTTTATTATTTCTCTTGCT
CTCATTTTTCACTACTCCAAGAAAGGCCAGAGAAGTTATA
AGTAA
Nucleic acid sequence of Homo sapiens
IL-10 polypeptide (SEQ ID NO: 16):
ATGCACAGCTCAGCACTGCTCTGTTGCCTGGTCCTCCTGA
CTGGGGTGAGGGCCAGCCCAGGCCAGGGCACCCAGTCTGA
GAACAGCTGCACCCACTTCCCAGGCAACCTGCCTAACATG
CTTCGAGATCTCCGAGATGCCTTCAGCAGAGTGAAGACTT
TCTTTCAAATGAAGGATCAGCTGGACAACTTGTTGTTAAA
GGAGTCCTTGCTGGAGGACTTTAAGGGTTACCTGGGTTGC
CAAGCCTTGTCTGAGATGATCCAGTTTTACCTGGAGGAGG
TGATGCCCCAAGCTGAGAACCAAGACCCAGACATCAAGGC
GCATGTGAACTCCCTGGGGGAGAACCTGAAGACCCTCAGG
CTGAGGCTACGGCGCTGTCATCGATTTCTTCCCTGTGAAA
ACAAGAGCAAGGCCGTGGAGCAGGTGAAGAATGCCTTTAA
TAAGCTCCAAGAGAAAGGCATCTACAAAGCCATGAGTGAG
TTTGACATCTTCATCAACTACATAGAAGCCTACATGACAA
TGAAGATACGAAACTGA
Nucleic acid sequence of Mus musculus IL-10
polypeptide (SEQ ID NO: 17):
ATGCCTGGCTCAGCACTGCTATGCTGCCTGCTCTTACTGA
CTGGCATGAGGATCAGCAGGGGCCAGTACAGCCGGGAAGA
CAATAACTGCACCCACTTCCCAGTCGGCCAGAGCCACATG
CTCCTAGAGCTGCGGACTGCCTTCAGCCAGGTGAAGACTT
TCTTTCAAACAAAGGACCAGCTGGACAACATACTGCTAAC
CGACTCCTTAATGCAGGACTTTAAGGGTTACTTGGGTTGC
CAAGCCTTATCGGAAATGATCCAGTTTTACCTGGTAGAAG
TGATGCCCCAGGCAGAGAAGCATGGCCCAGAAATCAAGGA
GCATTTGAATTCCCTGGGTGAGAAGCTGAAGACCCTCAGG
ATGCGGCTGAGGCGCTGTCATCGATTTCTCCCCTGTGAAA
ATAAGAGCAAGGCAGTGGAGCAGGTGAAGAGTGATTTTAA
TAAGCTCCAAGACCAAGGTGTCTACAAGGCCATGAATGAA
TTTGACATCTTCATCAACTGCATAGAAGCATACATGATGA
TCAAAATGAAAAGCTAA
Amino acid sequence of Homo sapiens
LIF polypeptide (SEQ ID NO: 18):
MKVLAAGVVPLLLVLHWKHGAGSPLPITPVNATCAIRHPC
HNNLMNQIRSQLAQLNGSANALFILYYTAQGEPFPNNLDK
LCGPNVTDFPPFHANGTEKAKLVELYRIVVYLGTSLGNIT
RDQKILNPSALSLHSKLNATADILRGLLSNVLCRLCSKYH
VGHVDVTYGPDTSGKDVFQKKKLGCQLLGKYKQIAVLAQA
F
Amino acid sequence of Mus musculus
LIF polypeptide (SEQ ID NO: 19):
MKVLAAGIVPLLHALHWKHGAGSPLPITPVNATCAIRHPC
HGNIMNQIKNQIAQLNGSANALFISYYTAQGEPFPNNVEK
LCAPNMTDFPSFHGNGTEKTKLVELYRMVAYLSASLTNIT
RDQKVLNPTAVSLQVKLNATIDVMRGLLSNVLCRLCNKYR
VGHVDVPPVPDHSDKEAFQRKKLGCQLLGTYKQVISWVQA
F
Amino acid sequence of Rattus norvegicus
LIP polypeptide (SEQ ID NO: 20):
MKVLATGIVPLLLILHWKHGAGSPLPITPVNATCAIRHPC
HGNLMNQIKSQLAQLNGSANALFISYYTAQGEPFPNNVDK
LCAPNMTDFPPFHANGTEKTKLV
ELYRMVAYLGASLTNITWDQKNLNPTAVSLOIKLNATTDV
MRGLLSNVLCRLCNKYHVGHVDVPCVPDNSSKEAFQRKKL
GCQLLGTYKQVISAVVQAF
Amino acid sequence of Canis lupus familiaris
LIF polypeptide (SEQ ID NO: 21):
MKVLAAGVVPLLLVLHWKHGAGSPLPITPVNATCATRHPC
HSNLMNQIRNQLAQLNGSANALHLYYTAQGEPHPNNLDKL
CGPNVTDHPPFHANSTEKTRLVELYRDAYLGASLGNITRD
QKVLNPNALSLHSKLNATADIMRGLLSNVLCRLCSKYHVA
HVDVAYGPDTSGKDVFQKKKLGCQLLGKYKQVIAWAQAF
Amino acid sequence ofMeriones unguiculatus
LIF polypeptide (SEQ ID NO: 22):
MKVLAAGVVPLLLVLHWKHGAGSPLPITPVNATCATRHPC
HGNLMHQIKNQLAQLNGSANALFISYYTAQGEPFPNNLDK
LCGPNMTDFPPFHANGTEKSKLVELYRMVAYLGASLGNVT
RDQKVLNPNALSLHGKLNATIDVMRGLLNNVLCRLCNKYH
VGHVDVTYAPDTSSKEIFQKKKAGCQLLGTYKQVISVVAQ
AF
Amino acid sequence of Ailuropoda melatnoleuca
LIF polypeptide (SEQ ID NO: 23):
MKVLAAGVVPLLLVLHWKHGAGSPLPITPYNATOATRHPC
HSNLMNQIRNQLAQLNGSANALFILYYTAQGEPFPNNLDK
LCGPNVTDFPPFHANGTERTRLVELYRLIAYLGASLGNIT
RDQKVLNPNAISLHSKLNATADIMRGLISNVLCRLCINKY
HVAHVDVAYGPDTSGKDVFQKKKIGCQLLGKYKQVIAVVA
QAF
Amino acid sequence of Odobenus rosmarus divergens
LIF polypeptide (SEQ ID NO: 24):
MKVLAAGVVPLLLVLHWKHGAGSPLPITPVNATCATRHPC
HSNLMNQIRNQLAQLNGSANALFILYYTAQGEPFPNNLDK
LCGPNVTDFPPFHANGTEKTRLVELYRIIAYLGASLGNIT
RDQKVLNPNALSLHSKLNATADIMRGLLSNVLCRLCNKYH
VAHVDVAYGPDTSGKDVFQKKKLGCQLLGKYKQVIAVVAQ
AF
Amino acid sequence of Pteropus vampyrus
LIF polypeptide (SEQ ID NO: 25):
MKVLAAGVVPLLLVLHWKHGAGSPLPITPVNATCVTRHPC
HNNLMNQIRNQLAQLNSSANALFILYYTAQGEPFPNNLDK
LCGPNVTDFPPFHANGTEKARLVELYRIIAYLGASLGNIT
RDQKILNPNALSLHSKLNATTDTMRGLLSNVLCRLCSKYH
VAHVEVAYGPDTSGKDVFQKKKLGCQLLGKYKQVIAVVAQ
AF
Amino acid sequence of Nannospalax galili
LIF polypeptide (SEQ ID NO: 26):
MKVLAAGVVPLLLVLHWKHGAASPLPITPVNATCATRHPC
HGNLMNQIKNQLAQLNGSANALFISYYTAQGEPFPNNLDK
LCGPNMTDFPPFHANGTEKAKLVELYRLVAYLGASLGNVT
RDQKILNPNALSLHSKLNATTDTMRGLLSNVLCRLCNKYH
VGHVDVTYGPDTAGKDAFQKKKLGCQLLGTYKQVISLVAQ
AF
Amino acid sequence of Homo sapiens
Klotho polypeptide (SEQ ID NO: 27):
MPASAPPRRPRPPPPSLSLLLVLLSGLGGRRLRAEPGDGA
QTWARFSRPPAPEAAGLPQGTFPDGFLWAVGSAAYQTEGG
WQQHGKGASIWDTFTHHPLAPPGDSRNASLPLGAPSPLQP
ATGDVASDSYNNVFRDTEALRELGVTHYRFSISWARVLPN
GSAGVPNREGLRYYRRLLERLRELGVQPVVTLYHWDLPQR
LQDAYGGWANRALADHFRDYAELCFRHFGGQVKYWITIDN
PYVVAWHGYATGRLAPGIRGSPRLGYLVAHNLLLAHAKVW
HLYNTSFRPTQGGQVSIALSSHWINPRRMTDHSIKECQKS
LDFVLGWFAKPVFIDGDYPESMKNNLSSILPDFTESEKKF
IKGTADFFALCFGPTLSFQLLDPHMKFRQLESPNLRQLLS
WIDLEFNHPQIFIVENGWFVSGTTKRDDAKYMYYLKKFIM
ETLKAIKLDGVDVIGYTAWSLMDGFEWHRGYSIRRGLFYV
DFLSQDKMLLPKSSALFYQKLIEKNGFPPLPENQPLEGTF
PCDFAWGVVDNYIQVDTTLSQFTDLNVYLWDVHHSKRLIK
VDGVVTKKRKSYCVDFAAIQPQIALLQEMHVTHFRFSLDW
ALILPLGNQSQVNHTILQYYRCMASELVRVNITPVVALWQ
PMAPNQGLPRLLARQGAWENPYTALAFAEYARLCFQELGH
HVKLWITMNEPYTRNMTYSAGHNLLKAHALAWHVYNEKFR
HAQNGKISIALQADWIEPACPFSQKDKEVAERVLEFDIGW
LAEPIFGSGDYPWVMRDWLNQRNNFLLPYFTEDEKKLIQG
TFDFLALSHYTTILVDSEKEDPIKYNDYLEVQEMTDITWL
NSPSQVAVVPWGLRKVLNWLKFKYGDLPMYIISNGIDDGL
HAEDDQLRVYYMQNYINEALKAHILDGINLCGYFAYSFND
RTAPRFGLYRYAADQFEPKASMKHYRKIIDSNGFPGPETL
ERFCPEEFTVCTECSFFHTRKSLLAFIAFLFFASIISLSL
IFYYSKKGRRSYK
Amino acid sequence of Mus musculus
Klotho polypeptide (SEQ ID NO: 28):
MLARAPPRRPPRLVLLRLLLLHLLLLALRARCLSAEPGQG
AQTWARFARAPAPEAAGLLHDTFPDGFLWAVGSAAYQTEG
GWRQHGKGASIWDTFTHHSGAAPSDSPIVVAPSGAPSPPL
SSTGDVASDSYNNVYRDTEGLRELGVTHYRFSISWARVLP
NGTAGTPNREGLRYYRRLLERLRELGVQPVVTLYHWDLPQ
RLQDTYGGWANRALADHFRDYAELCFRHFGGQVKYWITID
NPYVVAWHGYATGRLAPGVRGSSRLGYLVAHNLLLAHAKV
WHLYNTSFRPTQGGRVSIALSSHWINPRRMTDYNIRECQK
SLDFVLGWFAKPIFIDGDYPESMKNNLSSLLPDFTESEKR
LIRGTADFFALSFGPTLSFQLLDPNMKFRQLESPNLRQLL
SWIDLEYNHPPIFIVENGWFVSGTTKRDDAKYMYYLKKFI
METLKAIRLDGVDVIGYTAWSLMDGFEWHRGYSIRRGLFY
VDFLSQDKELLPKSSALFYQKLIEDNGFPPLPENQPLEGT
FPCDFAWGVVDNYVQVDTTLSQPTDPNVYLWDVHHSKRLI
KVDGVVAKKRKPYCVDFSAIRPQITLLREMRVTHFRFSLD
WALILPLGNQTQVNHTVLHFYRCMISELVHANITPVVALW
QPAAPHQGLPHALAKHGAWENPHTALAFADYANLCFKELG
HWVNLWITMNEPNTRNMTYRAGHHLLRAHALAWHLYDDKF
RAAQKGKISIALQADWIEPACPFSQNDKEVAERVLEFDIG
WLAEPIFGSGDYPRVMRDWLNQKNNFLLPYFTEDEKKLVR
GSFDFLAVSHYTTILVDWEKEDPMKYNDYLEVQEMTDITW
LNSPSQVAVVPWGLRKVLNWLRFKYGDLPMYVTANGIDDD
PHAEQDSLRIYYIKNYVNEALKAYVLDDINLCGYFAYSLS
DRSAPKSGFYRYAANQFEPKPSMKHYRKIIDSNGFLGSGT
LGRFCPEEYTVCTECGFFQTRKSLLVFISFLVFTFIISLA
LIFHYSKKGQRSYK
Amino acid sequence of Homo sapiens
IL-10 polypeptide (SEQ ID NO: 29):
MHSSALLCCLVLLTGVRASPGQGTQSENSCTHFPGNLPNM
LRDLRDAFSRVKTFFQMKDQLDNLILKESILEDFKGYLGC
QALSEMIQFYLEEVMPQAENQDPDIKAHVNSLGENLKTIR
IRLRRCHRFLPCENKSKAVEQVKNAFNKLQEKGIYKAMSE
FDIFINYIEAYMTMKIRN
Amino acid sequence of Mus musculus
IL-10 polypeptide (SEQ ID NO: 30):
MPGSALLCCLLLLTGMRISRGQYSREDNNCTHFPVGQSHM
LLELRTAFSQVKTFFQTKDQLDNILITDSIMQDFKGYLGC
QALSEMIQFYLVEVMPQAEKHGPEIKEHLNSLGEKIKTLR
MRLRRCHRFLPCENKSKAVEQVKSDFNKLQDQGVYKAMNE
FDIFINCIEAYMMIKMKS
Nucleic acid sequence of linker sequence
(SEQ ID NO: 31):
AAGCTTGGCATTCCGGTACTGTTGGTAAAGCCACCATGTAC

What is claimed is:

1. A polynucleotide comprising a CD11b promoter operably linked to a nucleic acid molecule encoding a therapeutic polypeptide.

2. The polynucleotide of claim 1, wherein the CD11b promoter is a human CD11b promotor or a chimpanzee CD11b promoter.

3. The polynucleotide of claim 1 or claim 2, wherein the CD11b promoter comprises a sequence at least 95%, at least 96%, at least 97% at least 98%, at least 99%, or 100% identical to the sequence of SEQ ID NO: 1 or 2.

4. The polynucleotide of any one of claim 1-3, wherein the nucleic acid molecule encodes a Leukemia Inhibitory Factor (LIF) polypeptide.

5. The polynucleotide of claim 4, wherein the nucleic acid molecule encoding the LIF polypeptide comprises a nucleic acid sequence at least 95%, at least 96%, at least 97%, at least 98%, at least 99%, or 100% identical to a sequence selected from the group consisting of SEQ ID NOS: 3-11.

6. The polynucleotide of claim 4 or claim 5, wherein the LIF polypeptide comprises an amino acid sequence at least 95%, at least 96%, at least 97%, at least 98%, at least 99%, or 100% identical to a sequence selected from the group consisting of SEQ ID NOS: 18-26.

7. The polynucleotide of any one of claims 4-6, wherein the polynucleotide comprises a sequence at least 95%, at least 96%, at least 97%, at least 98%, at least 99%, or 100% identical to the sequence of SEQ ID NOS: 12 or 13.

8. The polynucleotide of any one of claim 1-3, wherein the nucleic acid molecule encodes a Klotho polypeptide.

9. The polynucleotide of claim 8, wherein the nucleic acid molecule encoding the Klotho polypeptide comprises a nucleic acid sequence at least 95%, at least 96%, at least 97%, at least 98%, at least 99%, or 100% identical to the sequence of SEQ ID NOS: 14 or 15.

10. The polynucleotide of claim 8 or claim 9, wherein the Klotho polypeptide comprises an amino acid sequence at least 95%, at least 96%, at least 97%, at least 98%, at least 99%, or 100% identical to the sequence of SEQ ID NOS: 27 or 28.

11. The polynucleotide of any one of claim 1-3, wherein the nucleic acid molecule encodes an IL-10 polypeptide.

12. The polynucleotide of claim 11, wherein the nucleic acid molecule encoding the IL-10 polypeptide comprises a nucleic acid sequence at least 95%, at least 96%, at least 97%, at least 98%, at least 99%, or 100% identical to the sequence of SEQ ID NOS: 16 or 17.

13. The polynucleotide of claim 11 or claim 12, wherein the IL-10 polypeptide comprises an amino acid sequence at least 95%, at least 96%, at least 97%, at least 98%, at least 99%, or 100% identical to the sequence of SEQ ID NOS: 29 or 30.

14. The polynucleotide of any one of claims 1-13, wherein the polynucleotide further comprises a linker sequence between the CD11b promoter and the nucleic acid encoding the therapeutic polypeptide.

15. The polynucleotide of claim 14, wherein the linker sequence comprises a nucleic acid sequence at least 95%, at least 96%, at least 97%, at least 98%, at least 99%, or 100% identical to the sequence of SEQ ID NO: 31.

16. A vector comprising the polynucleotide of any one of claims 1-15.

17. The vector of claim 16, wherein the vector is a viral vector.

18. The vector of claim 17, wherein the viral vector is an adenoviral vector or a lentiviral vector.

19. The vector of any one of claims 16-18, wherein the vector is an expression vector.

20. A population of cells comprising the polynucleotide of any one of claims 1-15 or the vector of any one of claims 16-19.

21. The population of cells of claim 20, wherein the population of cells comprises hematopoietic stein cells.

22. The population of cells of claim 20 or claim 21, wherein the population of cells comprises primary cells isolated from a subject.

23. The population of cells of claim 22, wherein the primary cells are hematopoietic stem cells.

24. A composition comprising the polynucleotide of any one of claims 1-15, the vector of any one of claims 16-19, or the population of cells of any one of claims 20-23, and a pharmaceutically acceptable carrier.

25. The composition of claim 24, wherein the composition further comprises an immunosuppressant.

26. The composition of claim 25, wherein the immunosuppressant is selected from the group consisting of prednisone, deflazacort, and cytotoxic T-lymphocyte-associated protein-4.

27. A method of treating a subject in need thereof, the method comprising administering an effective amount of a population of cells comprising the polynucleotide of any one of claims 1-15 or the vector of any one of claims 16-19 to the subject.

28. The method of claim 27, wherein the population of cells comprises cells isolated from a healthy donor.

29. The method of claim 27, wherein the population of cells comprises cells isolated from the subject.

30. The method of any one of claims 27-29, wherein the population of cells comprises hematopoietic stem cells.

31. The method of any one of claims 27-30, wherein the population of cells are contacted with the polynucleotide or vector ex vivo.

32. The method of any one of claims 27-31, wherein at least one cell in the population of cells expresses the therapeutic polypeptide.

33. The method of any one of claims 27-32, wherein the method further comprises administering an immunosuppressant to the subject.

34. The method of any one of claims 27-33, wherein the subject suffers from a disease or condition selected from the group consisting of muscular dystrophy, polymyositis, dermatomyositis, multiple sclerosis, and autoimmune demyelination.

35. The method of claim 34, wherein administration of the population of cells reduces one or more signs or symptoms of the disease or condition in the subject.

36. The method of any one of claims 27-35, wherein administration of the population of cells reduces inflammation in the subject.

37. The method of any one of claims 27-36, wherein administration of the population of cells reduces fibrosis in the subject.

38. The method of any one of claims 27-37, wherein one or more cells of the population of cells localizes to a site of inflammation in the subject.

39. The method of claim 38, wherein the one or more cells are myeloid cells.

40. The method of claim 39, wherein the myeloid cells are selected from the group consisting of megakaryocytes, thrombocytes, erythrocytes, mast cells, myeloblasts, basophils, neutrophils, eosinophils, monocytes, macrophages, and any combinations thereof.


SEQUENCE LISTING (101 sequences)

SEQ ID NO: 1 - DNA, 550 bp, Homo sapiens
tgcagcctggacctcgggctcaagtaattctcacacctcagcctgtccagtagcaggggctacaggcgcgcaccaccatgcccagctaattaaaaatatttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctgttcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgca

SEQ ID NO: 2 - DNA, 213 bp, Pan troglodytes
tgcagcctccacttccgaggctcaagcaatcctcctgcctcagcctccccatagcagggaccacaggcacacaccaccacacccggctaatttttaaatttttttgtagagaagggatcttgctatgttgcccaggctggttcaaactcctgggttcaagcaatcctcctgccttggcctcccaaagtgctgggattacaggagtgagccact

SEQ ID NO: 3 - DNA, 609 bp, Homo sapiens
atgaaggtcttggcggcaggagttgtgcccctgctgttggttctgcactggaaacatggggcggggagccccctccccatcacccctgtcaacgccacctgtgccatacgccacccatgtcacaacaacctcatgaaccagatcaggagccaactggcacagctcaatggcagtgccaatgccctctttattctctattacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttctag

SEQ ID NO: 4 - DNA, 612 bp, Mus musculus
atgaaggtcttggccgcagggattgtgcccttactgctgctggttctgcactggaaacacggggcagggagccctcttcccatcacccctgtaaatgccacctgtgccatacgccacccatgccacggcaacctcatgaaccagatcaagaatcaactggcacagctcaatggcagcgccaatgctctcttcatttcctattacacagctcaaggggagccgtttcccaacaacgtggaaaagctatgtgcgcctaacatgacagacttcccatctttccatggcaacgggacagagaagaccaagttggtggagctgtatcggatggtcgcatacctgagcgcctccctgaccaatatcacccgggaccagaaggtcctgaaccccactgccgtgagcctccaggtcaagctcaatgctactatagacgtcatgaggggcctcctcagcaatgtgctttgccgtctgtgcaacaagtaccgtgtgggccacgtggatgtgccacctgtccccgaccactctgacaaagaagccttccaaaggaaaaagttgggttgccagcttctggggacatacaagcaagtcataagtgtggtggtccaggccttctag

SEQ ID NO: 5 - DNA, 609 bp, Rattus norvegicus
atgaaggtcttggccacagggattgtgcccctactgctcattctgcactggaaacacggggcagggagcccccttcccatcacccctgtaaatgccacctgcgccatacgccacccgtgtcacggcaacctcatgaaccagatcaagagtcaactggctcaactcaacggcagtgccaatgccctctttatttcctattacacagctcaaggggaaccatttcccaacaacgtggataagctatgtgcgcccaacatgacggatttcccacctttccatgccaatgggacagagaagaccaagttggtcgagctgtatcggatggtcgcgtacctgggagcctccctgaccaacatcacctgggatcagaaaaacctcaaccccactgccgtgagcctccagatcaaactcaatgcgactacagacgtcatgagggggctccttagcaacgtgctttgccgtctgtgcaacaagtaccatgtgggccatgtggatgtgccctgtgtccccgacaactctagcaaagaagccttccaaaggaagaagttgggctgccagctcctggggacatacaagcaagtcattagtgcggtggtccaggccttctag

SEQ ID NO: 6 - DNA, 609 bp, Canis lupus familiaris
atgaaggtcttggcggcaggagttgtgcccctgctgctggttctgcactggaaacacggggcagggagcccccttcctatcacccctgtcaacgccacctgcgccacacgccacccatgtcacagcaacctcatgaaccagatcaggaaccaactggcgcagctcaatggcagcgccaatgccctctttattctctattacacagcccagggggagccgttccccaacaacctggacaagctgtgtggccccaacgtgacagacttcccaccattccacgccaacagcacggagaagacccggctggtggagctgtaccgcatcatcgcctaccttggggcctccctgggcaacatcacccgggaccagaaggtcctcaatcccaatgccctcagcctccacagcaagctgaatgccactgcggacatcatgcggggcctcctcagcaacgtgctctgccgcctgtgtagcaagtaccacgtggcccacgtggacgtggcctatgggcccgacacctcgggcaaggatgtcttccagaagaagaagttgggctgtcagctcctggggaagtataagcaggtcattgccgtggtggcccaggccttctag

SEQ ID NO: 7 - DNA, 609 bp, Meriones unguiculatus
atgaaggtcttggccgcaggggttgtgcccttactgctggttctgcactggaaacacggggctgggagcccccttcccatcacccccgttaatgccacctgtgccacacgccacccatgtcatggcaacctcatgcaccagattaagaatcaactggctcagctcaatggcagtgccaatgccctcttcatctcctattacacagctcaaggggagccatttcccaacaacctggacaagctgtgtgggcctaacatgacagatttcccgcctttccacgccaacgggacggagaagagcaagttggtggagctgtatcggatggtcgcgtacctgggtgcctccctgggcaacgtcacccgggatcagaaggtcctcaaccccaacgccctgagcctccacggcaaactcaacgccactatcgacgtcatgcgcggcctcctcaacaatgtgctctgtcgcctgtgcaacaagtaccacgtcggccacgtggacgtgacgtacgcccccgacacctccagcaaagagatcttccaaaagaaaaaggcgggctgccagctcctagggacgtacaagcaagtcattagcgtggtggcccaggccttctag

SEQ ID NO: 8 - DNA, 609 bp, Ailuropoda melanoleuca
atgaaggtcttggcggcaggagttgtgcccctgctgctggttctgcactggaaacacggggcggggagcccccttcctatcacccctgtcaacgccacctgcgccacacgccacccatgtcacagcaacctcatgaaccagatcaggaaccaactggcgcagctcaatggcagtgccaatgctctctttattctctattacacagcccagggggagccgttccccaacaacctggacaagctgtgcggccccaacgtgacggacttcccgccattccacgccaacggcacggagaggacgcggctcgtggagttgtaccgcctcatcgcgtacctcggcgcctccctgggcaacatcacccgggaccagaaggtcctcaatcccaacgccctcagcctccacagcaagctgaacgccactgcggacatcatgcggggcctcctcagcaacgtgctctgccgcctgtgtaacaagtaccacgtggcccacgtggacgtggcctacggccccgacacctcgggcaaggacgtcttccagaagaagaagttgggctgtcagctcctggggaagtataagcaggtcatcgccgtggtggcccaggccttctag

SEQ ID NO: 9 - DNA, 609 bp, Odobenus rosmarus divergens
atgaaggtcttggcggcaggagttgtgcccctgctgctggttctgcactggaaacacggggcggggagcccccttcctatcacccctgtcaacgccacctgtgccacacgccacccatgtcacagcaacctcatgaaccagatcaggaaccaactggcgcagctcaatggcagtgccaatgctctctttattctctattacacagcccagggggagccgttccccaacaacctggacaagctgtgtggccccaatgtgacggacttcccgccattccacgccaacggcacggagaagacgcggctagtggagctgtaccgcatcatcgcgtacctcggcgcctccctgggcaacatcacccgggaccagaaggtcctcaatcccaacgccctcagcctccatagcaagctgaatgccactgcggacatcatgcggggcctccttagcaacgtgctctgccgcctgtgtaacaagtaccacgtggcccacgtggacgtggcctacggccccgacacctcgggcaaggacgtcttccagaagaagaagttgggctgtcagctcctggggaagtataagcaggtcatcgctgtggtggctcaggccttctag

SEQ ID NO: 10 - DNA, 609 bp, Pteropus vampyrus
atgaaggtcttggcagcaggagtcgtccccctgctgctggttctgcactggaaacacggggcagggagcccccttcccatcacccctgtcaacgccacctgtgtcacacgccacccatgtcacaacaacctcatgaaccagatcaggaaccaactggcccagctcaacagcagtgccaatgccctcttcattctctattacacggcccaaggggagccattccccaacaacctggacaagctgtgtggccccaacgtgacagacttcccacccttccatgccaatggcacggaaaaggcccggctggtggagctgtaccgcatcatcgcgtaccttggcgcctccctgggcaacatcacacgggaccagaagatcctcaatcccaacgccctcagcctccacagcaagctgaatgccaccacagacaccatgcggggcctcctcagtaacgtgctatgccgcctgtgcagcaagtaccacgtggcccacgtggaggtggcctatggccctgacacctcaggcaaggatgtcttccagaagaagaaattgggctgtcagctcctggggaagtataagcaggtcatcgctgtagtggcccaggccttctag

SEQ ID NO: 11 - DNA, 609 bp, Nannospalax galili
atgaaggtcttggccgcaggagttgtgcccttgctgctggttctgcactggaaacacggggcagcgagcccccttcccatcacccctgttaatgctacctgtgccacacgccacccatgtcatggcaacctcatgaatcagatcaagaaccaattggctcagctcaatggcagtgccaatgccctcttcatttcctattatacagctcagggggagccattccccaacaacctcgacaagctgtgtggacccaacatgacggacttcccacccttccatgccaatggcacagaaaaggccaagctggtggagctgtatcgtttggtcgcatacctgggtgcctccctgggcaacgtcacccgggatcagaagatcctgaaccccaatgccctgagcctccacagcaagctcaatgccaccacagacaccatgcggggcctcctcagcaatgtgctttgtcgcctgtgcaacaagtaccatgtgggccacgtggacgtgacctatggccctgacaccgcaggcaaggatgctttccaaaagaaaaagctgggctgccagctcctggggacgtacaagcaagtcattagcctggtggcccaggccttctag

SEQ ID NO: 12 - DNA, 1203 bp, Homo sapiens
tgcagcctggacctcgggctcaagtaattctcacacctcagcctgtccagtagcaggggctacaggcgcgcaccaccatgcccagctaattaaaaatatttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctgttcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgcaaagcttggcattccggtactgttggtaaagccaccatgtacatgaaggtcttggccgcagggattgtgcccttactgctgctggttctgcactggaaacacggggcagggagccctcttcccatcacccctgtaaatgccacctgtgccatacgccacccatgccacggcaacctcatgaaccagatcaagaatcaactggcacagctcaatggcagcgccaatgctctcttcatttcctattacacagctcaaggggagccgtttcccaacaacgtggaaaagctatgtgcgcctaacatgacagacttcccatctttccatggcaacgggacagagaagaccaagttggtggagctgtatcggatggtcgcatacctgagcgcctccctgaccaatatcacccgggaccagaaggtcctgaaccccactgccgtgagcctccaggtcaagctcaatgctactatagacgtcatgaggggcctcctcagcaatgtgctttgccgtctgtgcaacaagtaccgtgtgggccacgtggatgtgccacctgtccccgaccactctgacaaagaagccttccaaaggaaaaagttgggttgccagcttctggggacatacaagcaagtcataagtgtggtggtccaggccttctag

SEQ ID NO: 13 - DNA, 1200 bp, Homo sapiens
tgcagcctggacctcgggctcaagtaattctcacacctcagcctgtccagtagcaggggctacaggcgcgcaccaccatgcccagctaattaaaaatatttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctgttcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgcaaagcttggcattccggtactgttggtaaagccaccatgtacatgaaggtcttggcggcaggagttgtgcccctgctgttggttctgcactggaaacatggggcggggagccccctccccatcacccctgtcaacgccacctgtgccatacgccacccatgtcacaacaacctcatgaaccagatcaggagccaactggcacagctcaatggcagtgccaatgccctctttattctctattacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttctag

SEQ ID NO: 14 - DNA, 3039 bp, Homo sapiens
atgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatag

SEQ ID NO: 15 - DNA, 3045 bp, Mus musculus
atgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcaagtggacactactctctctcagtttactgacccgaatgtctatctgtgggatgtgcatcacagtaagaggcttattaaagtagacggggttgtagccaagaagagaaaaccttactgtgttgatttctctgccatccggcctcagataaccttacttcgagaaatgcgggtcacccactttcgcttctccctggactgggccctgatcttgcctctgggtaaccagacccaagtgaaccacacggttctgcacttctaccgctgcatgatcagcgagctggtgcacgccaacatcactccagtggtggccctgtggcagccagcagccccgcaccaaggcctgccacatgcccttgcaaaacatggggcctgggagaacccgcacactgctctggcgtttgcagactacgcaaacctgtgttttaaagagttgggtcactgggtcaatctctggatcaccatgaacgagccaaacacacggaacatgacctatcgtgccgggcaccacctcctgagagcccatgccttggcttggcatctgtacgatgacaagtttagggcggctcagaaaggcaaaatatccatcgccttgcaggctgactggatagaaccggcctgccctttctctcaaaatgacaaagaagtggccgagagagttttggaatttgatataggctggctggcagagcctatttttggttccggagattatccacgtgtgatgagggactggctgaaccaaaaaaacaattttcttttgccctatttcaccgaagatgaaaaaaagctagtccggggttcctttgacttcctggcggtgagtcattacaccaccattctggtagactgggaaaaggaggatccgatgaaatacaacgattacttggaggtacaggagatgactgacatcacatggctcaactctcccagtcaggtggcagtggtgccttgggggctgcgcaaagtgctcaactggctaaggttcaagtacggagacctcccgatgtatgtgacagccaatggaatcgatgatgacccccacgccgagcaagactcactgaggatctattatattaagaattatgtgaatgaggctctgaaagcctacgtgttggacgacatcaacctttgtggctactttgcgtattcacttagtgatcgctcagctcccaagtctggcttttatcgatatgctgcgaatcagtttgagcccaaaccatctatgaaacattacaggaaaattattgacagcaatggcttcctgggttctggaacactgggaaggttttgtccagaagaatacactgtgtgcaccgaatgtggattttttcaaacccggaagtctttgctggtcttcatctcgtttcttgtttttacttttattatttctcttgctctcatttttcactactccaagaaaggccagagaagttataagtaa

SEQ ID NO: 16 - DNA, 537 bp, Homo sapiens
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 17 - DNA, 537 bp, Mus musculus
atgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaa

SEQ ID NO: 18 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 19 - protein, 203 aa, Mus musculus [sequence omitted]

SEQ ID NO: 20 - protein, 202 aa, Rattus norvegicus
MKVLATGIVPLLLILHWKHGAGSPLPITPVNATCAIRHPCHGNLMNQIKSQLAQLNGSANALFISYYTAQGEPFPNNVDKLCAPNMTDFPPFHANGTEKTKLVELYRMVAYLGASLTNITWDQKNLNPTAVSLQIKLNATTDVMRGLLSNVLCRLCNKYHVGHVDVPCVPDNSSKEAFQRKKLGCQLLGTYKQVISAVVQAF

SEQ ID NO: 21 - protein, 202 aa, Canis lupus familiaris
MKVLAAGVVPLLLVLHWKHGAGSPLPITPVNATCATRHPCHSNLMNQIRNQLAQLNGSANALFILYYTAQGEPFPNNLDKLCGPNVTDFPPFHANSTEKTRLVELYRIIAYLGASLGNITRDQKVLNPNALSLHSKLNATADIMRGLLSNVLCRLCSKYHVAHVDVAYGPDTSGKDVFQKKKLGCQLLGKYKQVIAVVAQAF

SEQ ID NO: 22 - protein, 202 aa, Meriones unguiculatus
MKVLAAGVVPLLLVLHWKHGAGSPLPITPVNATCATRHPCHGNLMHQIKNQLAQLNGSANALFISYYTAQGEPFPNNLDKLCGPNMTDFPPFHANGTEKSKLVELYRMVAYLGASLGNVTRDQKVLNPNALSLHGKLNATIDVMRGLLNNVLCRLCNKYHVGHVDVTYAPDTSSKEIFQKKKAGCQLLGTYKQVISVVAQAF

SEQ ID NO: 23 - protein, 202 aa, Ailuropoda melanoleuca
MKVLAAGVVPLLLVLHWKHGAGSPLPITPVNATCATRHPCHSNLMNQIRNQLAQLNGSANALFILYYTAQGEPFPNNLDKLCGPNVTDFPPFHANGTERTRLVELYRLIAYLGASLGNITRDQKVLNPNALSLHSKLNATADIMRGLLSNVLCRLCNKYHVAHVDVAYGPDTSGKDVFQKKKLGCQLLGKYKQVIAVVAQAF

SEQ ID NO: 24 - protein, 202 aa, Odobenus rosmarus divergens
MKVLAAGVVPLLLVLHWKHGAGSPLPITPVNATCATRHPCHSNLMNQIRNQLAQLNGSANALFILYYTAQGEPFPNNLDKLCGPNVTDFPPFHANGTEKTRLVELYRIIAYLGASLGNITRDQKVLNPNALSLHSKLNATADIMRGLLSNVLCRLCNKYHVAHVDVAYGPDTSGKDVFQKKKLGCQLLGKYKQVIAVVAQAF

SEQ ID NO: 25 - protein, 202 aa, Pteropus vampyrus
MKVLAAGVVPLLLVLHWKHGAGSPLPITPVNATCVTRHPCHNNLMNQIRNQLAQLNSSANALFILYYTAQGEPFPNNLDKLCGPNVTDFPPFHANGTEKARLVELYRIIAYLGASLGNITRDQKILNPNALSLHSKLNATTDTMRGLLSNVLCRLCSKYHVAHVEVAYGPDTSGKDVFQKKKLGCQLLGKYKQVIAVVAQAF

SEQ ID NO: 26 - protein, 202 aa, Nannospalax galili
MKVLAAGVVPLLLVLHWKHGAASPLPITPVNATCATRHPCHGNLMNQIKNQLAQLNGSANALFISYYTAQGEPFPNNLDKLCGPNMTDFPPFHANGTEKAKLVELYRLVAYLGASLGNVTRDQKILNPNALSLHSKLNATTDTMRGLLSNVLCRLCNKYHVGHVDVTYGPDTAGKDAFQKKKLGCQLLGTYKQVISLVAQAF

SEQ ID NO: 27 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 28 - protein, 1014 aa, Mus musculus
MLARAPPRRPPRLVLLRLLLLHLLLLALRARCLSAEPGQGAQTWARFARAPAPEAAGLLHDTFPDGFLWAVGSAAYQTEGGWRQHGKGASIWDTFTHHSGAAPSDSPIVVAPSGAPSPPLSSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWHLYNTSFRPTQGGRVSIALSSHWINPRRMTDYNIRECQKSLDFVLGWFAKPIFIDGDYPESMKNNLSSLLPDFTESEKRLIRGTADFFALSFGPTLSFQLLDPNMKFRQLESPNLRQLLSWIDLEYNHPPIFIVENGWFVSGTTKRDDAKYMYYLKKFIMETLKAIRLDGVDVIGYTAWSLMDGFEWHRGYSIRRGLFYVDFLSQDKELLPKSSALFYQKLIEDNGFPPLPENQPLEGTFPCDFAWGVVDNYVQVDTTLSQFTDPNVYLWDVHHSKRLIKVDGVVAKKRKPYCVDFSAIRPQITLLREMRVTHFRFSLDWALILPLGNQTQVNHTVLHFYRCMISELVHANITPVVALWQPAAPHQGLPHALAKHGAWENPHTALAFADYANLCFKELGHWVNLWITMNEPNTRNMTYRAGHHLLRAHALAWHLYDDKFRAAQKGKISIALQADWIEPACPFSQNDKEVAERVLEFDIGWLAEPIFGSGDYPRVMRDWLNQKNNFLLPYFTEDEKKLVRGSFDFLAVSHYTTILVDWEKEDPMKYNDYLEVQEMTDITWLNSPSQVAVVPWGLRKVLNWLRFKYGDLPMYVTANGIDDDPHAEQDSLRIYYIKNYVNEALKAYVLDDINLCGYFAYSLSDRSAPKSGFYRYAANQFEPKPSMKHYRKIIDSNGFLGSGTLGRFCPEEYTVCTECGFFQTRKSLLVFISFLVFTFIISLALIFHYSKKGQRSYK

SEQ ID NO: 29 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 30 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 31 - DNA, 41 bp, Artificial Sequence
  Synthetic Construct
aagcttggcattccggtactgttggtaaagccaccatgtac

SEQ ID NO: 32 - DNA, 26 bp, Artificial Sequence
  Synthetic Construct
catgaatgatgcttacctgggttatg

SEQ ID NO: 33 - DNA, 19 bp, Artificial Sequence
  Synthetic Construct
gtcttggccgcagggattg

SEQ ID NO: 34 - DNA, 20 bp, Artificial Sequence
  Synthetic Construct
gacccatgacctgcagaaac

SEQ ID NO: 35 - DNA, 21 bp, Artificial Sequence
  Synthetic Construct
caaagcttctgctgtggaaat

SEQ ID NO: 36 - DNA, 21 bp, Artificial Sequence
  Synthetic Construct
cagcacaggaaatgtttcagc

SEQ ID NO: 37 - DNA, 18 bp, Artificial Sequence
  Synthetic Construct
gcaaaaactggcagtggg

SEQ ID NO: 38 - DNA, 21 bp, Artificial Sequence
  Synthetic Construct
ggattgtggagcagatggaag

SEQ ID NO: 39 - DNA, 21 bp, Artificial Sequence
  Synthetic Construct
caatgaagagctggctggtgt

SEQ ID NO: 40 - DNA, 23 bp, Artificial Sequence
  Synthetic Construct
gaagtggttagtagagctgtgtc

SEQ ID NO: 41 - DNA, 22 bp, Artificial Sequence
  Synthetic Construct
cttctgtctactgaacttcggg

SEQ ID NO: 42 - DNA, 21 bp, Artificial Sequence
  Synthetic Construct
gacaatcaggccatcagcaac

SEQ ID NO: 43 - DNA, 20 bp, Artificial Sequence
  Synthetic Construct
gtaatgaaagacggcacacc

SEQ ID NO: 44 - DNA, 21 bp, Artificial Sequence
  Synthetic Construct
gaacaacgatgatgcacttgc

SEQ ID NO: 45 - DNA, 21 bp, Artificial Sequence
  Synthetic Construct
tgccttggtagcatctatgag

SEQ ID NO: 46 - DNA, 19 bp, Artificial Sequence
  Synthetic Construct
ggatgtgccaaacgtcctc

SEQ ID NO: 47 - DNA, 20 bp, Artificial Sequence
  Synthetic Construct
caaggagcatttgaattccc

SEQ ID NO: 48 - DNA, 18 bp, Artificial Sequence
  Synthetic Construct
ctccacctgcaagaccat

SEQ ID NO: 49 - DNA, 21 bp, Artificial Sequence
  Synthetic Construct
ctttcttatccgcgacagctc

SEQ ID NO: 50 - DNA, 22 bp, Artificial Sequence
  Synthetic Construct
cctgtaaatgccatgcaagttc

SEQ ID NO: 51 - DNA, 21 bp, Artificial Sequence
  Synthetic Construct
gctcagccagatgcagttaac

SEQ ID NO: 52 - DNA, 19 bp, Artificial Sequence
  Synthetic Construct
caaccagatgggcccaatg

SEQ ID NO: 53 - DNA, 21 bp, Artificial Sequence
  Synthetic Construct
gataaggctccagtcacctgc

SEQ ID NO: 54 - DNA, 19 bp, Artificial Sequence
  Synthetic Construct
ctggaccagatgcggtgag

SEQ ID NO: 55 - DNA, 20 bp, Artificial Sequence
  Synthetic Construct
tgtgtgcgatgacgtgcaat

SEQ ID NO: 56 - DNA, 23 bp, Artificial Sequence
  Synthetic Construct
atcccatttggagaatgttgtgc

SEQ ID NO: 57 - DNA, 20 bp, Artificial Sequence
  Synthetic Construct
cggggtactcctggtcctac

SEQ ID NO: 58 - DNA, 20 bp, Artificial Sequence
  Synthetic Construct
gacgcactgaccgacgattc

SEQ ID NO: 59 - DNA, 21 bp, Artificial Sequence
  Synthetic Construct
ggacacctaaaatcgccaagc

SEQ ID NO: 60 - DNA, 19 bp, Artificial Sequence
  Synthetic Construct
gctcagcaaatcgtgcagc

SEQ ID NO: 61 - DNA, 21 bp, Artificial Sequence
  Synthetic Construct
cttgtgtctgcacgacctgtg

SEQ ID NO: 62 - DNA, 21 bp, Artificial Sequence
  Synthetic Construct
ggagggcaagatggtcagtag

SEQ ID NO: 63 - DNA, 20 bp, Artificial Sequence
  Synthetic Construct
aggctcaccaggaatgtgac

SEQ ID NO: 64 - DNA, 21 bp, Artificial Sequence
  Synthetic Construct
cctgctgctctcaaggttgtt

SEQ ID NO: 65 - DNA, 19 bp, Artificial Sequence
  Synthetic Construct
agagcgagcagttcctgac

SEQ ID NO: 66 - DNA, 20 bp, Artificial Sequence
  Synthetic Construct
ggacccgagaagacctcctt

SEQ ID NO: 67 - DNA, 22 bp, Artificial Sequence
  Synthetic Construct
cccaaaataagagccaatctgg

SEQ ID NO: 68 - DNA, 22 bp, Artificial Sequence
  Synthetic Construct
cccaaaataagagccaatctgg

SEQ ID NO: 69 - DNA, 19 bp, Artificial Sequence
  Synthetic Construct
gaaggcagtggcatggaac

SEQ ID NO: 70 - DNA, 19 bp, Artificial Sequence
  Synthetic Construct
gactggtcacggttgcaag

SEQ ID NO: 71 - DNA, 19 bp, Artificial Sequence
  Synthetic Construct
tagccagcgtaccggatga

SEQ ID NO: 72 - DNA, 19 bp, Artificial Sequence
  Synthetic Construct
gtcaaaatcacagacggag

SEQ ID NO: 73 - DNA, 21 bp, Artificial Sequence
  Synthetic Construct
cttgaatggaaatgcacagac

SEQ ID NO: 74 - DNA, 21 bp, Artificial Sequence
  Synthetic Construct
gtgtgagcatccacccaaatg

SEQ ID NO: 75 - DNA, 23 bp, Artificial Sequence
  Synthetic Construct
ggtgagaggtgtattaatgtccg

SEQ ID NO: 76 - DNA, 21 bp, Artificial Sequence
  Synthetic Construct
cacttggtggtttgctacgac

SEQ ID NO: 77 - DNA, 22 bp, Artificial Sequence
  Synthetic Construct
cggatgagctcattgaatgctt

SEQ ID NO: 78 - DNA, 19 bp, Artificial Sequence
  Synthetic Construct
ctctgcagactcaaactcc

SEQ ID NO: 79 - DNA, 26 bp, Artificial Sequence
  Synthetic Construct
cttcatgtactccaggtagctatggt

SEQ ID NO: 80 - DNA, 20 bp, Artificial Sequence
  Synthetic Construct
ttcaggcggagctcagatag

SEQ ID NO: 81 - DNA, 22 bp, Artificial Sequence
  Synthetic Construct
gagttcttcttcaagcatggag

SEQ ID NO: 82 - DNA, 21 bp, Artificial Sequence
  Synthetic Construct
ggccttgtagacaccttggtc

SEQ ID NO: 83 - DNA, 21 bp, Artificial Sequence
  Synthetic Construct
cttagtttggacaggatctgg

SEQ ID NO: 84 - DNA, 21 bp, Artificial Sequence
  Synthetic Construct
cactggatgcgtaggttcttg

SEQ ID NO: 85 - DNA, 21 bp, Artificial Sequence
  Synthetic Construct
gtatgccgtggatgaactgag

SEQ ID NO: 86 - DNA, 21 bp, Artificial Sequence
  Synthetic Construct
ctctctcttgagcttggtgac

SEQ ID NO: 87 - DNA, 22 bp, Artificial Sequence
  Synthetic Construct
gataacagcttcccagggacac

SEQ ID NO: 88 - DNA, 20 bp, Artificial Sequence
  Synthetic Construct
ccctgcttggtctggaaaac

SEQ ID NO: 89 - DNA, 19 bp, Artificial Sequence
  Synthetic Construct
aagatcacagcttcccggg

SEQ ID NO: 90 - DNA, 19 bp, Artificial Sequence
  Synthetic Construct
gggtccctcgactcctaca

SEQ ID NO: 91 - DNA, 23 bp, Artificial Sequence
  Synthetic Construct
ggacatgattcacagattccagg

SEQ ID NO: 92 - DNA, 20 bp, Artificial Sequence
  Synthetic Construct
gcatccctacttcccccttg

SEQ ID NO: 93 - DNA, 21 bp, Artificial Sequence
  Synthetic Construct
ctgcgatgcatctctctctgg

SEQ ID NO: 94 - DNA, 22 bp, Artificial Sequence
  Synthetic Construct
ggcacaggtcttgatgaacatc

SEQ ID NO: 95 - DNA, 22 bp, Artificial Sequence
  Synthetic Construct
ctaggtaggtccgttcccactg

SEQ ID NO: 96 - DNA, 19 bp, Artificial Sequence
  Synthetic Construct
gtcagcaaaagcacggttg

SEQ ID NO: 97 - DNA, 20 bp, Artificial Sequence
  Synthetic Construct
cggtgactactgtgctttcg

SEQ ID NO: 98 - DNA, 20 bp, Artificial Sequence
  Synthetic Construct
cttggccatcaatttgtcct

SEQ ID NO: 99 - DNA, 21 bp, Artificial Sequence
  Synthetic Construct
cgatagtgcatcttggccttt

SEQ ID NO: 100 - DNA, 20 bp, Artificial Sequence
  Synthetic Construct
cggtgctgatcttccttttc

SEQ ID NO: 101 - DNA, 19 bp, Artificial Sequence
  Synthetic Construct
gctgccgttgtcaaacacc